SEQUENCE LISTING (6 sequences)

SEQ ID NO: 1 - protein, 448 aa, Homo sapiens
VYFPAQYVDQTDLEKYNNVEAGKYTVGLGQTRMGFCSVQEDINSLCLTVVQRLMERIQLPWDSVGRLEVGTETIIDKSKAVKTVLMELFQDSGNTDIEGIDTTNACYGGTASLFNAANWMESSSWDGRYAMVVCGDIAVYPSGNARPTGGAGAVAMLIGPKAPLALERGLRGTHMENVYDFYKPNLASEYPIVDGKLSIQCYLRALDRCYTSYRKKIQNQWKQAGSDRPFTLDDLQYMIFHTPFCKMVQKSLARLMFNDFLSASSDTQTSLYKGLEAFGGLKLEDTYTNKDLDKALLKASQDMFDKKTKASLYLSTHNGNMYTSSLYGCLASLLSHHSAQELAGSRIGAFSYGSGLAASFFSFRVSQDAAPGSPLDKLVSSTSDLPKRLASRKCVSPEEFTEIMNQREQFYHKVNFSPPGDTNSLFPGTWYLERVDEQHRRKYARRPV

SEQ ID NO: 2 - DNA, 2433 bp, Homo sapiens
  source 1..2433: mol_type = genomic DNA
atctctttcaaggtttctgctgggtttctgaactgctgggtttctgcttgctcctctggagatgcagcgtctgttgactccagtgaagcgcattctgcaactgacaagagcggtgcaggaaacctccctcacacctgctcgcctgctcccagtagcccaccaaaggttttctacagcctctgctgtccccctggccaaaacagatacttggccaaaggacgtgggcatcctggccctggaggtctacttcccagcccaatatgtggaccaaactgacctggagaagtataacaatgtggaagcaggaaagtatacagtgggcttgggccagacccgtatgggcttctgctcagtccaagaggacatcaactccctgtgcctgacggtggtgcaacggctgatggagcgcatacagctcccatgggactctgtgggcaggctggaagtaggcactgagaccatcattgacaagtccaaagctgtcaaaacagtgctcatggaactcttccaggattcaggcaatactgatattgagggcatagataccaccaatgcctgctacggtggtactgcctccctcttcaatgctgccaactggatggagtccagttcctgggatggtcgttatgccatggtggtctgtggagacattgccgtctatcccagtggtaatgctcgtcccacaggtggggccggagctgtggctatgctgattgggcccaaggcccctctggccctggagcgagggctgaggggaacccatatggagaatgtgtatgacttctacaaaccaaatttggcctcggagtacccaatagtggatgggaagctttccatccagtgctacttgcgggccttggatcgatgttacacatcataccgtaaaaaaatccagaatcagtggaagcaagctggcagcgatcgacccttcacccttgacgatttacagtacatgatctttcatacacccttttgcaagatggtccagaagtctctggctcgcctgatgttcaatgacttcctgtcagccagcagtgacacacaaaccagcttatataaggggctggaggctttcggggggctaaagctggaagacacctacaccaacaaggacctggataaagcacttctaaaggcctctcaggacatgttcgacaagaaaaccaaggcttccctttacctctccactcacaatgggaacatgtacacctcatccctgtacgggtgcctggcctcgcttctgtcccaccactctgcccaagaactggctggctccaggattggtgccttctcttatggctctggtttagcagcaagtttcttttcatttcgagtatcccaggatgctgctccaggctctcccctggacaagttggtgtccagcacatcagacctgccaaaacgcctagcctcccgaaagtgtgtgtctcctgaggagttcacagaaataatgaaccaaagagagcaattctaccataaggtgaatttctccccacctggtgacacaaacagccttttcccaggtacttggtacctggagcgagtggacgagcagcatcgccgaaagtatgcccggcgtcccgtctaaaggtgttctgcagatccatggaaagcttcctgggaaacgtatgctagcagagcttctccccgtgaatcatatttttaagatcccactcttagctggtaaatgaatttgaatcgacatagtagccccataagcatcagccctgtagagtgaggagccatctctagcgggcccttcattcctctccatgctgcaatcactgtcctgggcttatggtgctatggactaggggtcctttgtgaaagagcaagatggagcaatggagagaagacctcttcctgaatcactggactccagaaatgtgcatgcagatcagctgttgccttcaagatccagataaactttcctgtcatgtgttagaactttattattattaatattgttaaacttctgtgctgttcctgtgaatctccaaattttgtaccttgttctaagctaatatatagcaattaaaaagagagaaagaggaaatgattcctgcgtttcttggaacccagaatacaaacccagcctaacatgcagcaagcctgctagaccttgtgggtcagagggctgggtccttgcctcacaggctgcctctgtccccttgcaattccattctatttctgccacatgccaagtgctatgacaggtacaaggcaaataagaacggtagaacacagcttcccccagcccacttccctgttctaaagacaccacatagacagagagcagcagacaggggccagcaggagctgtagttcagatcttcttggtcattccttgccgctgttatttgaacaaataaacacagcgcaaaggttaacaagtttttgccttctatagccaaaaataaaaaaataaataaa

SEQ ID NO: 3 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 4 - DNA, 2258 bp, Homo sapiens
  source 1..2258: mol_type = genomic DNA
gcagccccgggcgccgcgcgtcctgcccggcctgcggccccagcccttgcgccgctcgtccgacccgcgatcgtccaccagaccgtgcctcccggccgcccggccggcccgcgtgcatgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacgtagccccgcgatgccagaggaggggcactgggtggggagggaggtggaggagccgtgcaatcccaaccaggatgtctagcacccccatccccttggcccttcctcatgcttctgaagtggacatattcagccttggcgagaagctccgttgcacgggtttccccttgagcccattttacagatgaggaaactgagtccggagaggaaaagggacatggctcccgtgcactagcttgttacagctgcctctgtccccacatgtgggggcaccttctccagtaggattcggaaaagattgtacatatgggaggagggggcagattcctggccctccctccccagacttgaaggtggggggtaggttggttgttcagagtcttcccaataaagatgagtttttgagcc

SEQ ID NO: 5 - protein, 474 aa, Homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 6 - DNA, 4869 bp, Homo sapiens
  source 1..4869: mol_type = genomic DNA
gctctaagctgcagcaagagaaactgtgtgtgaggggaagaggcctgtttcgctgtcgggtctctagttcttgcacgctctttaagagtctgcactggaggaactcctgccattaccagctcccttcttgcagaagggagggggaaacatacatttattcatgccagtctgttgcatgcaggctttttggcttcctaccttgcaacaaaataattgcaccaactccttagtgccgattccgcccacagagagtcctggagccacagtcttttttgctttgcattgtaggagagggactaagtgctagagactatgtcgctttcctgagctaccgagagcgctcgtgaactggaatcaactgcttcagggaaaaagaaaaaaaaaaaaaaaagacttgcctgggaggccgcgagaaacttgcattggaagcttcagcaaccagcattcgagaaactcctctctactttagcacggtctccagactcagccgagagacagcaaactgcagcgcggtgagagagcgagagagagggagagagagactctccagcctgggaactataactcctctgcgagaggcggagaactccttccccaaatcttttggggacttttctctctttacccacctccgcccctgcgaggagttgaggggccagttcggccgccgcgcgcgtcttcccgttcggcgtgtgcttggcccggggaaccgggagggcccggcgatcgcgcggcggccgccgcgagggtgtgagcgcgcgtgggcgcccgccgagccgaggccatggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactgaagggcgcgcaggcagggagaagggccggggggggtaggagaggagaaaaaaaaagtgaaaaaaagaaacgaaaaggacagacgaagagtttaaagagaaaagggaaaaaagaaagaaaaagtaagcagggctggcttcgcccgcgttctcgtcgtcggatcaaggagcgcggcggcgttttggacccgcgctcccatcccccaccttcccgggccggggacccactctgcccagccggagggacgcggaggaggaagagggtagacaggggcgacctgtgattgttgttattgatgttgttgttgatggcaaaaaaaaaaaagcgacttcgagtttgctcccctttgcttgaagagaccccctcccccttccaacgagcttccggacttgtctgcacccccagcaagaaggcgagttagttttctagagacttgaaggagtctcccccttcctgcatcaccaccttggttttgttttattttgcttcttggtcaagaaaggaggggagaacccagcgcacccctccccccctttttttaaacgcgtgatgaagacagaaggctccggggtgacgaatttggccgatggcagatgttttgggggaacgccgggactgagagactccacgcaggcgaattcccgtttggggcttttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaagaaaaaaaaagaaaaaaaaaagatttttttcttctcttaatcggaatcgtgatggtgttggattatttcaatggtggggttaatatagcatgttatcctgtctatcttttaaagatttctgtataagactgttgagcagtttttaaaatagtgtaggataatataaaaagcagatagatggcgctatgtttgattcctacaacgaaattatcaccagctttttttcattcttaactctttaaaggattcaaacgcaactcaaatctgtgctggactttaaaaaaacaattcaggaccaaattttttctcagtgtgtgtgtttattccttataggtgtaaatgagaagacgtgtttttttccttcaccgatgctccatcctcgtatttctttttccttgtaaatgtaatcagatgccattttatatgtggacgtatttatactggccaaacatattttttcttttgtccctttttttctttcctttctttttacttcctttatttctttattccttccttttcctttttttcttttttttttctttttttttttttttttttggtagttgttgttacccacgccattttacgtctccttcactgaagggctagagttttaacttttaattttttatatttaaatgtagacttttgacacttttaaaaaacaaaaaaagacaagagagatgaaaacgtttgattattttctcagtgtatttttgtaaaaaatatataaagggggtgttaatcggtgtaaatcgctgtttggatttcctgattttataacagggcggctggttaatatctcacacagtttaaaaaatcagcccctaatttctccatgtttacacttcaatctgcaggcttcttaaagtgacagtatcccttaacctgccaccagtgtccaccctccggcccccgtcttgtaaaaaggggaggagaattagccaaacactgtaagcttttaagaaaaacaaagttttaaacgaaatactgctctgtccagaggctttaaaactggtgcaattacagcaaaaagggattctgtagctttaacttgtaaaccacatcttttttgcactttttttataagcaaaaacgtgccgtttaaaccactggatctatctaaatgccgatttgagttcgcgacactatgtactgcgtttttcattcttgtatttgactatttaatcctttctacttgtcgctaaatataattgttttagtcttatggcatgatgatagcatatgtgttcaggtttatagctgttgtgtttaaaaattgaaaaaagtggaaaacatctttgtacatttaagtctgtattataataagcaaaaagattgtgtgtatgtatgtttaatataacatgacaggcactaggacgtctgcctttttaaggcagttccgttaagggtttttgtttttaaacttttttttgccatccatcctgtgcaatatgccgtgtagaatatttgtcttaaaattcaaggccacaaaaacaatgtttgggggaaaaaaaagaaaaaatcatgccagctaatcatgtcaagttcactgcctgtcagattgttgatatataccttctgtaaataactttttttgagaaggaaataaaatcagctggaactgaaccctaaa

(19) United States
(12) Patent Application Publication
(10) Pub. No.: US 2026/0258507 A1
(43) Pub. Date: Sep. 3, 2026

(54) COMPOSITIONS AND METHODS FOR CHARACTERIZING HEPATOCYTE STRESS

(71) Applicants: Massachusetts Institute of Technology, Cambridge, MA (US); The General Hospital Corporation, Boston, MA (US); The Brigham and Women's Hospital, Inc., Boston, MA (US)

(72) Inventors: Wolfram Goessling, Boston, MA (US); Alexander K. Shalek, Cambridge, MA (US); Constantine N. Tzouanas, Cambridge, MA (US); Ömer H. Yilmaz, Cambridge, MA (US); Jessica E. Shay, Cambridge, MA (US); Marc S. Sherman, Boston, MA (US)

(73) Assignees: Massachusetts Institute of Technology, Cambridge, MA (US); The General Hospital Corporation, Boston, MA (US); The Brigham and Women's Hospital, Inc., Boston, MA (US)

(21) Appl. No.: 19/670,970

(22) Filed: May 7, 2026

Related U.S. Application Data
(63) Continuation of application No. PCT/US2024/056107, filed on Nov. 15, 2024.
(60) Provisional application No. 63/599,875, filed on Nov. 16, 2023.

(51) Int. Cl. C12Q 1/6886; C12Q 1/6883; G01N 33/57525; G01N 33/68

(52) U.S. Cl. CPC C12Q 1/6886; C12Q 1/6883; G01N 33/57525; G01N 33/6893; C12Q 2600/118; C12Q 2600/158; G01N 2800/085; G01N 2800/52

(57) ABSTRACT

The disclosure provides compositions and methods for identifying subjects having hepatocyte stress associated with a risk for developing hepatocellular carcinoma (HCC), and therapeutic methods for reducing a subject's risk of HCC, for example, by inhibiting RELB, SOX4, or HMGCS2.

### CROSS REFERENCE TO RELATED APPLICATIONS

This application is a continuation under 35 U.S.C. § 111 (a) of PCT International Patent Application No. PCT/US2024/056107, filed Nov. 15, 2024, designating the United States and published in English, which claims priority to and the benefit of U.S. Provisional Application No. 63/599,875, filed Nov. 16, 2023, the entire contents of each of which are incorporated by reference herein.

### SEQUENCE LISTING

The instant application contains a Sequence Listing which has been submitted electronically in XML format and is hereby incorporated by reference in its entirety. Said XML copy, created on Nov. 20, 2024, is named 167741-052301PCT_SL.xml and is 17,263 bytes in size.

### BACKGROUND OF THE DISCLOSURE

Cells must balance immediate viability with supporting tissue homeostasis and contributing to organismal health. The liver performs wide-ranging functions including nutrient metabolism, protein secretion, and chemical detoxification. Moreover, it possesses substantial regenerative capacity, restoring normal mass and function after acute challenges as dramatic as surgical removal of two-thirds of its tissue. However, this intrinsic regenerative ability proves insufficient or even detrimental during chronic stress, with the liver incapable of mitigating progressive tissue damage. For instance, chronic exposure to high caloric diets can precipitate metabolic dysfunction-associated steatotic liver disease (MASLD, formerly NAFLD/NASH; affecting ~25% of people around the world), which in turn drives progressive fibrosis, liver failure, and hepatocellular carcinoma (HCC; the second-leading cause of years of life lost to cancer).

Hepatocytes are the primary cells of the liver, executing most of its functions but also serving as the cell-of-origin for HCC. Epidemiological studies indicate that each successive MASLD stage associates with a progressive increase in HCC incidence. However, mutations mainly accumulate after cirrhosis, but not before: among pre-cancer liver disease patients, only patients with cirrhotic livers (but not earlier fibrosis stages) exhibited significant increases in mutation rate compared to patients with non-fibrotic livers. Among a MASLD-predominant cohort, convergent somatic mutations were enriched for metabolic enzymes, but largely did not overlap with HCC driver mutations. Improved methods of identifying subjects at risk for developing HCC, and for mitigating this risk are urgently required.

### SUMMARY

Featured and described herein are compositions and methods for identifying subjects having hepatocyte stress associated with a propensity to develop hepatocellular carcinoma (HCC), and therapeutic methods for treating and/or reducing a subject's risk of acquiring HCC, for example, by inhibiting RELB, SOX4, or HMGCS2.

In an aspect a method of selecting a subject for therapy is provided, in which the method involves detecting an increase in the expression or activity of a SOX4, RELB, and HMGCS2 polynucleotide or polypeptide; and administering to the subject an agent that inhibits SOX4, RelB, or HMGCS2 expression. In an embodiment of the method, the agent that inhibits Sox4 is an integrin avb6/8-blocking monoclonal antibody (mAb). In an embodiment, the agent is an inhibitory polynucleotide that targets Sox4. In an embodiment, the inhibitory polynucleotide is an siRNA, shRNA, or antisense polynucleotide. In an embodiment, the agent that inhibits RelB is 1,25-Dihydroxyvitamin D3 or vinorelbine. In an embodiment, the agent that inhibits RelB is an inhibitory polynucleotide. In an embodiment, the inhibitory polynucleotide is an siRNA, shRNA, or antisense polynucleotide. In an embodiment, the agent that inhibits HMGCS2 is momilactone B or an HMGCS2 inhibitor.

In an embodiment of the method, levels of the polypeptide or polynucleotide are increased. In an embodiment of the above-delineated method and/or embodiments thereof, RELB and SOX4 drive metabolic adaptation and tumor priming phenotypes. In an embodiment of the above-delineated method and/or embodiments thereof, the method further involves detecting increases in any one or more of the following markers or sets of markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, polynucleotides encoding those polypeptides;
viii. any marker listed as increased in Table 1 or any polypeptide listed as increased in Table 2A or Table 2B;
ix. a RELB, SOX4, JUND, ONECUT1, MAFF, RORC, DBP, or RXRA polypeptide or polynucleotide; and
x. a THRB, PPARA, NR1H4 polypeptide or polynucleotide.

In an embodiment of the above-delineated method and/or embodiments thereof, the method further involves detecting decreases in any one or more of the following sets of markers:
i. downregulation metabolic enzymes and secreted polypeptides Aspg, Hao1, Hamp, or polynucleotides encoding the polypeptides;
ii. suppressors of hepatocellular carcinoma and fetal hepatocyte-associated phenotypes iii. Gls2, Zbtb20, Esr1 polypeptides or polynucleotides encoding the polypeptides;
iii. decreases in hepatocyte secreted FGB and FABP1 polypeptides or polynucleotides encoding the polypeptides;
iv. polypeptides associated with hepatocyte cellular identity and function including NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides; and
v. any marker listed as decreased in Table 1 or any polypeptide listed as decreased in Table 2A or 2B.

In another aspect, a method of treating a subject for hepatocellular carcinoma, metabolic dysfunction-associated steatotic liver disease (MASLD), progressive fibrosis, and/or liver failure is provided, in which the method involves administering to the subject an agent that inhibits SOX4, RelB, or HMGCS2 expression.

In another aspect, a method of treating a selected subject having or having a propensity to develop hepatocellular carcinoma is provided, in which the method involves administering to the subject an agent listed in FIG. 23A, wherein the subject is selected by detecting an increase in the expression of a marker or set of markers defined in Table 3 or Table 4 as having a longitudinal increase, sustained upregulation, longitudinal decrease, sustained downregulation, development-associated hepatocyte, or Human HCC S1 Wnt Activator, and wherein the agent is selected as effective against a liver disease characterized by that marker or set of markers shown in FIG. 23A.

In an embodiment of the methods of the above-delineated aspects and/or embodiments thereof, the subject has metabolic dysfunction-associated steatotic liver disease (MASLD), progressive fibrosis, and/or liver failure.

In another aspect, a method of characterizing the prognosis of a subject having metabolic dysfunction-associated steatotic liver disease (MASLD) is provided, in which the method involves detecting an increase in the expression or activity of a SOX4, RELB, and/or HMGCS2 polynucleotide or polypeptide. In an embodiment of the method, the increase in the expression or activity of a SOX4, RELB, and/or HMGCS2 polypeptide or polynucleotide is associated with the development of hepatocellular carcinoma. In an embodiment of the method, the increase in the expression or activity of a SOX4, RELB, and/or HMGCS2 polypeptide or polynucleotide is associated with a poor prognosis. In an embodiment, the method further involves detecting increases in any one or more of the following sets of markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, or polynucleotides encoding those polypeptides;
viii. WNT/β-catenin target Axin2;
ix. WNT-associated polypeptides Tbx3, Axin2, Lgr5, Notum or polynucleotides encoding those polypeptides;
x. HCC-linked polypeptides Spp1, Igf2r or polynucleotides encoding those polypeptides;
or polynucleotides encoding those polypeptides;
xi. a RELB, SOX4, JUND, ONECUT1, MAFF, RORC, DBP, or RXRA polypeptide or polynucleotide encoding those polypeptides; and
xii. a THRB, PPARA, NR1H4 polypeptide or polynucleotide encoding those polypeptides.
   In an embodiment, the method further involves detecting decreases in any one or more of the following sets of markers:
i. downregulation metabolic enzymes and secreted polypeptides Aspg, Hao1, Hamp, or polynucleotides encoding the polypeptides;
ii. suppressors of hepatocellular carcinoma and fetal hepatocyte-associated phenotypes iii. Gls2, Zbtb20, Esr1 polypeptides or polynucleotides encoding the polypeptides;
iii. decreases in hepatocyte secreted FGB and FABP1 polypeptides or polynucleotides encoding the polypeptides;
iv. hepatocyte cellular identity polypeptides NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides; or
v. decreases in polypeptides associated with hepatocyte function Cps1, Pck1, Hamp, C4a, Pdia5 or polynucleotides encoding those polypeptides.

In another aspect, a panel of capture molecules comprising two or more molecules each of which specifically binds a SOX4, RELB, and/or HMGCS2 polypeptide or polynucleotide. In an embodiment, the panel further comprises one or more capture molecules each of which binds one of the following markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, or polynucleotides encoding those polypeptides;
viii. metabolic enzymes and secreted polypeptides Aspg, Hao1, Hamp, or polynucleotides encoding the polypeptides;
ix. suppressors of hepatocellular carcinoma and fetal hepatocyte-associated phenotypes Gls2, Zbtb20, Esr1 polypeptides or polynucleotides encoding the polypeptides;
x. hepatocyte secreted FGB and FABP1 polypeptides or polynucleotides encoding the polypeptides;
xi. polypeptides associated with hepatocyte cellular identity and function including NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides;
xii. decreases in polypeptides associated with hepatocyte function Cps1, Pck1, Hamp, C4a, Pdia5 or polynucleotides encoding those polypeptides;
xiii. a RELB, SOX4, JUND, ONECUT1, MAFF, RORC, DBP, or RXRA polypeptide or polynucleotide encoding those polypeptides; and
xiv. a THRB, PPARA, NR1H4 polypeptide or polynucleotide encoding those polypeptides.
   In an embodiment of the panel, the capture molecule is an antibody. In an embodiment, the capture molecule is a polynucleotide that hybridizes to a polynucleotide encoding a marker.

In another aspect, a kit for characterizing metabolic dysfunction-associated steatotic liver disease (MASLD) or hepatocellular carcinoma in a subject is provided, in which the kit comprises the capture molecule as described in the above-delineated aspect and/or embodiments thereof, and instructions for the use of the kit according to the method as described in any one of the above-delineated aspects and/or embodiments thereof.

Compositions and articles defined by the present disclosure were isolated or otherwise manufactured in connection with the disclosure and examples provided hereinbelow. Other features and advantages of the disclosure will be apparent from the detailed description, and from the claims.

### Definitions

Unless defined otherwise, all technical and scientific terms used herein have the meaning commonly understood by a person skilled in the art related to the present disclosure. The following references provide one of skill with a general definition of many of the terms used in the disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. Liver diseases associated with stress are stratified using the markers described herein (e.g., at Tables 3 and 4) and treated with the corresponding agents delineated in FIG. 23A and FIG. 23B. For example, markers identified under the header Longitudinal Increase in Table 3 are treated with the agents identified in FIGS. 23A and 23B as useful in the treatment of liver disease characterized by markers that show a longitudinal increase.

By “ameliorate” is meant decrease, suppress, attenuate, diminish, arrest, or stabilize the development or progression of a disease.

By “alteration” is meant a change (increase or decrease) in the expression levels, structure, or activity of a gene or polypeptide as detected by standard art known methods such as those described herein. As used herein, an alteration includes a 10% change in expression levels, a 25% change, a 40% change, or a 50% or greater change in expression levels.”

As used herein, the term “antisense strand” refers to a polynucleotide that is substantially or 100% complementary to a target nucleic acid of interest. For example, an antisense strand may be complementary, in whole or in part, to a molecule of mRNA (messenger RNA), an RNA sequence that is not mRNA (e.g., microRNA, piwiRNA, tRNA, rRNA and hnRNA) or a sequence of DNA that is either coding or non-coding. The terms “antisense strand” and “guide strand” are used interchangeably herein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In embodiments described herein, the disease is a liver disease associated with hepatocyte stress (e.g., hepatocellular carcinoma, metabolic dysfunction-associated steatotic liver disease (MASLD, formerly NAFLD/NASH).

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of a required to ameliorate the symptoms of a disease relative to an untreated patient. The effective amount of active compound(s) used to practice the described embodiments for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In embodiments, an effective amount of an agent described herein inhibits a marker delineated herein, or otherwise counteracts changes associated with hepatocyte stress, hepatocellular carcinoma or MASLD.

Provided are a number of targets that are useful for the development of highly specific drugs to treat or a disorder characterized by the methods delineated herein. In addition, the methods of the present disclosure provide a facile means to identify therapies that are safe for use in subjects. In addition, the methods of the disclosure provide a route for analyzing virtually any number of compounds for effects on a disease described herein with high-volume throughput, high sensitivity, and low complexity.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “high-throughput sequencing” is meant a sequencing technique that allows for large amounts of nucleic acids to be sequenced.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “3-hydroxy-3-methylglutaryl-CoA synthase 2 (HMGCS2) polypeptide” is meant a protein having at least about 85% amino acid sequence identity to Genbank Accession No. NCBI Reference Sequence No. NP_005509.1 for a fragment thereof having DNA binding activity. An exemplary HMGCS2 polypeptide is provided below:

(SEQ ID NO: 1)
61 vyfpaqyvdq tdlekynnve agkytvglgq trmgfcsvqe dinslcltvv qrlmeriqlp
121 wdsvgrlevg tetiidkska vktvlmelfq dsgntdiegi dttnacyggt aslfnaanwm
181 essswdgrya mvvcgdiavy psgnarptgg agavamligp kaplalergl rgthmenvyd
241 fykpnlasey pivdgklsiq cylraldrcy tsyrkkiqnq wkqagsdrpf tlddlqymif
301 htpfckmvqk slarlmfndf lsassdtqts lykgleafgg lkledtytnk dldkallkas
361 qdmfdkktka slylsthngn mytsslygcl asllshhsaq elagsrigaf sygsglaasf
421 fsfrvsqdaa pgspldklvs stsdlpkrla srkcvspeef teimnqreqf yhkvnfsppg
481 dtnslfpgtw ylervdeqhr rkyarrpv

By “3-hydroxy-3-methylglutaryl-CoA synthase 2 (HMGCS2) polynucleotide” is meant a nucleic acid molecule that encodes an HMGCS2 polypeptide. In embodiments, a HMGCS2 polynucleotide is the genomic sequence, cDNA, mRNA, or gene associated with and/or required for HMGCS2 expression. An exemplary HMGCS2 polynucleotide sequence is provided at NCBI Reference Sequence No. NM_005518.4, which is reproduced below:

```
(SEQ ID NO: 2)
      1 atctctttca aggtttctgc tgggtttctg aactgctggg tttctgcttg ctcctctgga
    61 gatgcagcgt ctgttgactc cagtgaagcg cattctgcaa ctgacaagag cggtgcagga
  121 aacctccctc acacctgctc gcctgctccc agtagcccac caaaggtttt ctacagcctc
  181 tgctgtcccc ctggccaaaa cagatacttg gccaaaggac gtgggcatcc tggccctgga
  241 ggtctacttc ccagcccaat atgtggacca aactgacctg gagaagtata acaatgtgga
  301 agcaggaaag tatacagtgg gcttgggcca gacccgtatg ggcttctgct cagtccaaga
  361 ggacatcaac tccctgtgcc tgacggtggt gcaacggctg atggagcgca tacagctccc
  421 atgggactct gtgggcaggc tggaagtagg cactgagacc atcattgaca agtccaaagc
  481 tgtcaaaaca gtgctcatgg aactcttcca ggattcaggc aatactgata ttgagggcat
  541 agataccacc aatgcctgct acggtggtac tgcctccctc ttcaatgctg ccaactggat
  601 ggagtccagt tcctgggatg gtcgttatgc catggtggtc tgtggagaca ttgccgtcta
  661 tcccagtggt aatgctcgtc ccacaggtgg ggccggagct gtggctatgc tgattgggcc
  721 caaggcccct ctggccctgg agcgagggct gaggggaacc catatggaga atgtgtatga
  781 cttctacaaa ccaaatttgg cctcggagta cccaatagtg gatgggaagc tttccatcca
  841 gtgctacttg cgggccttgg atcgatgtta cacatcatac cgtaaaaaaa tccagaatca
  901 gtggaagcaa gctggcagcg atcgaccctt cacccttgac gatttacagt acatgatctt
  961 tcatacaccc ttttgcaaga tggtccagaa gtctctggct cgcctgatgt tcaatgactt
1021 cctgtcagcc agcagtgaca cacaaaccag cttatataag gggctggagg ctttcggggg
1081 gctaaagctg gaagacacct acaccaacaa ggacctggat aaagcacttc taaaggcctc
1141 tcaggacatg ttcgacaaga aaaccaaggc ttccctttac ctctccactc acaatgggaa
1201 catgtacacc tcatccctgt acgggtgcct ggcctcgctt ctgtcccacc actctgccca
1261 agaactggct ggctccagga ttggtgcctt ctcttatggc tctggtttag cagcaagttt
1321 cttttcattt cgagtatccc aggatgctgc tccaggctct cccctggaca agttggtgtc
1381 cagcacatca gacctgccaa aacgcctagc ctcccgaaag tgtgtgtctc ctgaggagtt
1441 cacagaaata atgaaccaaa gagagcaatt ctaccataag gtgaatttct ccccacctgg
1501 tgacacaaac agccttttcc caggtacttg gtacctggag cgagtggacg agcagcatcg
1561 ccgaaagtat gcccggcgtc ccgtctaaag gtgttctgca gatccatgga aagcttcctg
1621 ggaaacgtat gctagcagag cttctccccg tgaatcatat ttttaagatc ccactcttag
1681 ctggtaaatg aatttgaatc gacatagtag ccccataagc atcagccctg tagagtgagg
1741 agccatctct agcgggccct tcattcctct ccatgctgca atcactgtcc tgggcttatg
1801 gtgctatgga ctaggggtcc tttgtgaaag agcaagatgg agcaatggag agaagacctc
1861 ttcctgaatc actggactcc agaaatgtgc atgcagatca gctgttgcct tcaagatcca
1921 gataaacttt cctgtcatgt gttagaactt tattattatt aatattgtta aacttctgtg
1981 ctgttcctgt gaatctccaa attttgtacc ttgttctaag ctaatatata gcaattaaaa
2041 agagagaaag aggaaatgat tcctgcgttt cttggaaccc agaatacaaa cccagcctaa
2101 catgcagcaa gcctgctaga ccttgtgggt cagagggctg ggtccttgcc tcacaggctg
2161 cctctgtccc cttgcaattc cattctattt ctgccacatg ccaagtgcta tgacaggtac
2221 aaggcaaata agaacggtag aacacagctt cccccagccc acttccctgt tctaaagaca
2281 ccacatagac agagagcagc agacaggggc cagcaggagc tgtagttcag atcttcttgg
2341 tcattccttg ccgctgttat ttgaacaaat aaacacagcg caaaggttaa caagtttttg
2401 ccttctatag ccaaaaataa aaaaataaat aaa
```

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” or “biomarker” is meant any protein or polynucleotide having an alteration in expression level or activity that is associated with a disease or disorder. Markers described herein include polynucleotides (e.g., mRNA) and polypeptides whose levels are altered in a biological sample of a subject having a liver disease (e.g., hepatocellular carcinoma, metabolic dysfunction-associated steatotic liver disease (MASLD, formerly NAFLD/NASH). Exemplary markers having an increase in expression associated with liver disease (e.g., hepatocellular carcinoma, metabolic dysfunction-associated steatotic liver disease (MASLD, formerly NAFLD/NASH) include, but are not limited to the following:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iii. development-associated and regeneration-linked polypeptides CD24, CDKN1A, and LGR5 or polynucleotides encoding those polypeptides;
iv. HCC-associated polypeptides p62/SQSTM1, CD151, LGALS1, CD74 or polynucleotides encoding those polypeptides;
v. CD24, LGR5 and NKD1; and
vi. genes with functional effects in HCC CD151, MDK, AIFM2, AKR1C2, ROBO1, or polynucleotides encoding those polypeptides.

In some embodiments, decreases in any one or more of the following markers is associated with liver disease:
i. downregulation metabolic enzymes and secreted polypeptides Aspg, Hao1, Hamp, or polynucleotides encoding the polypeptides;
ii. suppressors of hepatocellular carcinoma and fetal hepatocyte-associated phenotypes iii. Gls2, Zbtb20, Esr1 polypeptides or polynucleotides encoding the polypeptides;
iii. decreases in hepatocyte secreted FGB and FABP1 polypeptides or polynucleotides encoding the polypeptides;
iv. polypeptides associated with hepatocyte cellular identity and function including NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides.

By “portion” is meant a fragment of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

By “RNA-seq” is meant RNA sequencing for detecting and quantifying messenger RNA molecules (mRNA) in a biological sample, which, for example, may be used to study cellular responses. A related term, “scRNA-seq” is single-cell RNA sequencing, which may be, for example, a droplet-based single-cell RNA-seq or “Drop-seq,” that is a sequencing technology for analyzing RNA expression in at least hundreds of thousands of individual cells in embodiments of the disclosure, but may alternatively use any other high-throughput sequencing platform.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition. In embodiments, the level of a marker in a subject having liver disease is compared to the level of that marker in a healthy control subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides, or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, and less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In an embodiment, wash steps will occur at 25° C. in 30 mM NaCl, 3 mM trisodium citrate, and 0.1% SDS. In an embodiment, wash steps will occur at 42 C in 15 mM NaCl, 1.5 mM trisodium citrate, and 0.1% SDS. In an embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, or 80% or 85%, or 90%, 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “Transcription factor RELB (RELB) polypeptide” is meant a protein having at least about 85% amino acid sequence identity to NCBI Reference Sequence No. NP_006500.2 or a fragment thereof, and having DNA binding activity. An exemplary RELB polypeptide sequence is provided below:

>NP_006500. 2 transcription factor RelB isoform 1 [Homo sapiens]
(SEQ ID NO: 3)
MLRSGPASGPSVPTGRAMPSRRVARPPAAPELGALGSPDLSSLSLAVSRSTDELEIIDEYIKENGFGLDG
GQPGPGEGLPRLVSRGAASLSTVTLGPVAPPATPPPWGCPLGRLVSPAPGPGPQPHLVITEQPKORGMRF
RYECEGRSAGSILGESSTEASKTLPAIELRDCGGLREVEVTACLVWKDWPHRVHPHSLVGKDCTDGICRV
RLRPHVSPRHSFNNLGIQCVRKKEIEAAIERKIQLGIDPYNAGSLKNHQEVDMNVVRICFQASYRDQQGQ
MRRMDPVLSEPVYDKKSTNTSELRICRINKESGPCTGGEELYLLCDKVQKEDISVVFSRASWEGRADFSQ
ADVHRQIAIVFKTPPYEDLEIVEPVTVNVFLQRLTDGVCSEPLPFTYLPRDHDSYGVDKKRKRGMPDVLG
ELNSSDPHGIESKRRKKKPAILDHFLPNHGSGPFLPPSALLPDPDFFSGTVSLPGLEPPGGPDLLDDGFA
YDPTAPTLFTMLDLLPPAPPHASAVVCSGGAGAVVGETPGPEPLTLDSYQAPGPGDGGTASLVGSNMFPN
HYREAAFGGGLLSPGPEAT

By “Transcription factor RELB (RELB) polynucleotide” is meant a nucleic acid molecule that encodes an RELB polypeptide. In embodiments, a RELB polynucleotide is the genomic sequence, cDNA, mRNA, or gene associated with and/or required for RELB expression. An exemplary RELB polynucleotide sequence is provided at NCBI Reference Sequence No. NM_006509.4, which is reproduced below:

```
(SEQ ID NO: 4)
      1 gcagccccgg gcgccgcgcg tcctgcccgg cctgcggccc cagcccttgc gccgctcgtc
    61 cgacccgcga tcgtccacca gaccgtgcct cccggccgcc cggccggccc gcgtgcatgc
  121 ttcggtctgg gccagcctct gggccgtccg tccccactgg ccgggccatg ccgagtcgcc
  181 gcgtcgccag accgccggct gcgccggagc tgggggcctt agggtccccc gacctctcct
  241 cactctcgct cgccgtttcc aggagcacag atgaattgga gatcatcgac gagtacatca
  301 aggagaacgg cttcggcctg gacgggggac agccgggccc gggcgagggg ctgccacgcc
  361 tggtgtctcg cggggctgcg tccctgagca cggtcaccct gggccctgtg gcgcccccag
  421 ccacgccgcc gccttggggc tgccccctgg gccgactagt gtccccagcg ccgggcccgg
  481 gcccgcagcc gcacctggtc atcacggagc agcccaagca gcgcggcatg cgcttccgct
  541 acgagtgcga gggccgctcg gccggcagca tccttgggga gagcagcacc gaggccagca
  601 agacgctgcc cgccatcgag ctccgggatt gtggagggct gcgggaggtg gaggtgactg
  661 cctgcctggt gtggaaggac tggcctcacc gagtccaccc ccacagcctc gtggggaaag
  721 actgcaccga cggcatctgc agggtgcggc tccggcctca cgtcagcccc cggcacagtt
  781 ttaacaacct gggcatccag tgtgtgagga agaaggagat tgaggctgcc attgagcgga
  841 agattcaact gggcattgac ccctacaacg ctgggtccct gaagaaccat caggaagtag
  901 acatgaatgt ggtgaggatc tgcttccagg cctcatatcg ggaccagcag ggacagatgc
  961 gccggatgga tcctgtgctt tccgagcccg tctatgacaa gaaatccaca aacacatcag
1021 agctgcggat ttgccgaatt aacaaggaaa gcgggccgtg caccggtggc gaggagctct
1081 acttgctctg cgacaaggtg cagaaagagg acatatcagt ggtgttcagc agggcctcct
1141 gggaaggtcg ggctgacttc tcccaggccg acgtgcaccg ccagattgcc attgtgttca
1201 agacgccgcc ctacgaggac ctggagattg tcgagcccgt gacagtcaac gtcttcctgc
1261 agcggctcac cgatggggtc tgcagcgagc cattgccttt cacgtacctg cctcgcgacc
1321 atgacagcta cggcgtggac aagaagcgga aacgggggat gcccgacgtc cttggggagc
1381 tgaacagctc tgacccccat ggcatcgaga gcaaacggcg gaagaaaaag ccggccatcc
1441 tggaccactt cctgcccaac cacggctcag gcccgttcct cccgccgtca gccctgctgc
1501 cagaccctga cttcttctct ggcaccgtgt ccctgcccgg cctggagccc cctggcgggc
1561 ctgacctcct ggacgatggc tttgcctacg accctacggc ccccacactc ttcaccatgc
1621 tggacctgct gcccccggca ccgccacacg ctagcgctgt tgtgtgcagc ggaggtgccg
1681 gggccgtggt tggggagacc cccggccctg aaccactgac actggactcg taccaggccc
1741 cgggccccgg ggatggaggc accgccagcc ttgtgggcag caacatgttc cccaatcatt
1801 accgcgaggc ggcctttggg ggcggcctcc tatccccggg gcctgaagcc acgtagcccc
1861 gcgatgccag aggaggggca ctgggtgggg agggaggtgg aggagccgtg caatcccaac
1921 caggatgtct agcaccccca tccccttggc ccttcctcat gcttctgaag tggacatatt
1981 cagccttggc gagaagctcc gttgcacggg tttccccttg agcccatttt acagatgagg
2041 aaactgagtc cggagaggaa aagggacatg gctcccgtgc actagcttgt tacagctgcc
2101 tctgtcccca catgtggggg caccttctcc agtaggattc ggaaaagatt gtacatatgg
2161 gaggaggggg cagattcctg gccctccctc cccagacttg aaggtggggg gtaggttggt
2221 tgttcagagt cttcccaata aagatgagtt tttgagcc
```

By “Transcription factor SOX-4 (SOX-4) polypeptide” is meant a polypeptide having at least about 85% amino acid sequence identity to NCBI Reference Sequence No. NP_003098.1 or a fragment thereof having DNA binding activity. provided below, incorporated herein by reference in its entirety. An exemplary SOX-4 polypeptide sequence is provided below.

```
(SEQ ID NO: 5)
    1 mvqqtnnaen teallagess dsgaglelgi assptpgsta stggkaddps wcktpsghik
  61 rpmnafmvws qierrkimeq spdmhnaeis krlgkrwkll kdsdkipfir eaerlrlkhm
121 adypdykyrp rkkvksgnan ssssaaassk pgekgdkvgg sgggghgggg gggssnaggg
181 gggasgggan skpaqkkscg skvaggaggg vskphaklil aggggggkaa aaaaasfaae
241 qagaaallpl gaaadhhsly kartpsasas assaasasaa laapgkhlae kkvkrvylfg
301 glgtssspvg gvgagadpsd plglyeeega gcspdapsls grssaasspa agrspadhrg
361 yaslraaspa pssapshass sasshsssss ssgssssdde feddlldlnp ssnfesmslg
421 sfssssaldr dldfnfepgs gshfefpdyc tpevsemisg dwlessisnl vity
```

By “Transcription factor SOX-4 (SOX-4) polynucleotide” is meant a nucleic acid molecule that encodes an SOX4 polypeptide or fragment thereof having DNA binding activity. In embodiments, a SOX4 polynucleotide is the genomic sequence, cDNA, mRNA, or gene associated with and/or required for SOX4 expression. An exemplary SOX4 polynucleotide is provided at NCBI Reference Sequence No. NM_003107.3, which is reproduced below.

```
      1 gctctaagct gcagcaagag aaactgtgtg tgaggggaag aggcctgttt cgctgtcggg
    61 tctctagttc ttgcacgctc tttaagagtc tgcactggag gaactcctgc cattaccagc
  121 tcccttcttg cagaagggag ggggaaacat acatttattc atgccagtct gttgcatgca
  181 ggctttttgg cttcctacct tgcaacaaaa taattgcacc aactccttag tgccgattcc
  241 gcccacagag agtcctggag ccacagtctt ttttgctttg cattgtagga gagggactaa
  301 gtgctagaga ctatgtcgct ttcctgagct accgagagcg ctcgtgaact ggaatcaact
  361 gcttcaggga aaaagaaaaa aaaaaaaaaa agacttgcct gggaggccgc gagaaacttg
  421 cattggaagc ttcagcaacc agcattcgag aaactcctct ctactttagc acggtctcca
  481 gactcagccg agagacagca aactgcagcg cggtgagaga gcgagagaga gggagagaga
  541 gactctccag cctgggaact ataactcctc tgcgagaggc ggagaactcc ttccccaaat
  601 cttttgggga cttttctctc tttacccacc tccgcccctg cgaggagttg aggggccagt
  661 tcggccgccg cgcgcgtctt cccgttcggc gtgtgcttgg cccggggaac cgggagggcc
  721 cggcgatcgc gcggcggccg ccgcgagggt gtgagcgcgc gtgggcgccc gccgagccga
  781 ggccatggtg cagcaaacca acaatgccga gaacacggaa gcgctgctgg ccggcgagag
  841 ctcggactcg ggcgccggcc tcgagctggg aatcgcctcc tcccccacgc ccggctccac
  901 cgcctccacg ggcggcaagg ccgacgaccc gagctggtgc aagaccccga gtgggcacat
  961 caagcgaccc atgaacgcct tcatggtgtg gtcgcagatc gagcggcgca agatcatgga
1021 gcagtcgccc gacatgcaca acgccgagat ctccaagcgg ctgggcaaac gctggaagct
1081 gctcaaagac agcgacaaga tccctttcat tcgagaggcg gagcggctgc gcctcaagca
1141 catggctgac taccccgact acaagtaccg gcccaggaag aaggtgaagt ccggcaacgc
1201 caactccagc tcctcggccg ccgcctcctc caagccgggg gagaagggag acaaggtcgg
1261 tggcagtggc gggggcggcc atgggggcgg cggcggcggc gggagcagca acgcgggggg
1321 aggaggcggc ggtgcgagtg gcggcggcgc caactccaaa ccggcgcaga aaaagagctg
1381 cggctccaaa gtggcgggcg gcgcgggcgg tggggttagc aaaccgcacg ccaagctcat
1441 cctggcaggc ggcggcggcg gcgggaaagc agcggctgcc gccgccgcct ccttcgccgc
1501 cgaacaggcg ggggccgccg ccctgctgcc cctgggcgcc gccgccgacc accactcgct
1561 gtacaaggcg cggactccca gcgcctcggc ctccgcctcc tcggcagcct cggcctccgc
1621 agcgctcgcg gccccgggca agcacctggc ggagaagaag gtgaagcgcg tctacctgtt
1681 cggcggcctg ggcacgtcgt cgtcgcccgt gggcggcgtg ggcgcgggag ccgaccccag
1741 cgaccccctg ggcctgtacg aggaggaggg cgcgggctgc tcgcccgacg cgcccagcct
1801 gagcggccgc agcagcgccg cctcgtcccc cgccgccggc cgctcgcccg ccgaccaccg
1861 cggctacgcc agcctgcgcg ccgcctcgcc cgccccgtcc agcgcgccct cgcacgcgtc
1921 ctcctcggcc tcgtcccact cctcctcttc ctcctcctcg ggctcctcgt cctccgacga
1981 cgagttcgaa gacgacctgc tcgacctgaa ccccagctca aactttgaga gcatgtccct
2041 gggcagcttc agttcgtcgt cggcgctcga ccgggacctg gattttaact tcgagcccgg
2101 ctccggctcg cacttcgagt tcccggacta ctgcacgccc gaggtgagcg agatgatctc
2161 gggagactgg ctcgagtcca gcatctccaa cctggttttc acctactgaa gggcgcgcag
2221 gcagggagaa gggccggggg gggtaggaga ggagaaaaaa aaagtgaaaa aaagaaacga
2281 aaaggacaga cgaagagttt aaagagaaaa gggaaaaaag aaagaaaaag taagcagggc
2341 tggcttcgcc cgcgttctcg tcgtcggatc aaggagcgcg gcggcgtttt ggacccgcgc
2401 tcccatcccc caccttcccg ggccggggac ccactctgcc cagccggagg gacgcggagg
2461 aggaagaggg tagacagggg cgacctgtga ttgttgttat tgatgttgtt gttgatggca
2521 aaaaaaaaaa agcgacttcg agtttgctcc cctttgcttg aagagacccc ctcccccttc
2581 caacgagctt ccggacttgt ctgcaccccc agcaagaagg cgagttagtt ttctagagac
2641 ttgaaggagt ctcccccttc ctgcatcacc accttggttt tgttttattt tgcttcttgg
2701 tcaagaaagg aggggagaac ccagcgcacc cctccccccc tttttttaaa cgcgtgatga
2761 agacagaagg ctccggggtg acgaatttgg ccgatggcag atgttttggg ggaacgccgg
2821 gactgagaga ctccacgcag gcgaattccc gtttggggct tttttttcct ccctcttttc
2881 cccttgcccc ctctgcagcc ggaggaggag atgttgaggg gaggaggcca gccagtgtga
2941 ccggcgctag gaaatgaccc gagaaccccg ttggaagcgc agcagcggga gctaggggcg
3001 ggggcggagg aggacacgaa ctggaagggg gttcacggtc aaactgaaat ggatttgcac
3061 gttggggagc tggcggcggc ggctgctggg cctccgcctt cttttctacg tgaaatcagt
3121 gaggtgagac ttcccagacc ccggaggcgt ggaggagagg agactgtttg atgtggtaca
3181 ggggcagtca gtggagggcg agtggtttcg gaaaaaaaaa aagaaaaaaa gaaaaaaaaa
3241 gaaaaaaaaa agattttttt cttctcttaa tcggaatcgt gatggtgttg gattatttca
3301 atggtggggt taatatagca tgttatcctg tctatctttt aaagatttct gtataagact
3361 gttgagcagt ttttaaaata gtgtaggata atataaaaag cagatagatg gcgctatgtt
3421 tgattcctac aacgaaatta tcaccagctt tttttcattc ttaactcttt aaaggattca
3481 aacgcaactc aaatctgtgc tggactttaa aaaaacaatt caggaccaaa ttttttctca
3541 gtgtgtgtgt ttattcctta taggtgtaaa tgagaagacg tgtttttttc cttcaccgat
3601 gctccatcct cgtatttctt tttccttgta aatgtaatca gatgccattt tatatgtgga
3661 cgtatttata ctggccaaac atattttttc ttttgtccct ttttttcttt cctttctttt
3721 tacttccttt atttctttat tccttccttt tccttttttt cttttttttt tctttttttt
3781 tttttttttt tggtagttgt tgttacccac gccattttac gtctccttca ctgaagggct
3841 agagttttaa cttttaattt tttatattta aatgtagact tttgacactt ttaaaaaaca
3901 aaaaaagaca agagagatga aaacgtttga ttattttctc agtgtatttt tgtaaaaaat
3961 atataaaggg ggtgttaatc ggtgtaaatc gctgtttgga tttcctgatt ttataacagg
4021 gcggctggtt aatatctcac acagtttaaa aaatcagccc ctaatttctc catgtttaca
4081 cttcaatctg caggcttctt aaagtgacag tatcccttaa cctgccacca gtgtccaccc
4141 tccggccccc gtcttgtaaa aaggggagga gaattagcca aacactgtaa gcttttaaga
4201 aaaacaaagt tttaaacgaa atactgctct gtccagaggc tttaaaactg gtgcaattac
4261 agcaaaaagg gattctgtag ctttaacttg taaaccacat cttttttgca ctttttttat
4321 aagcaaaaac gtgccgttta aaccactgga tctatctaaa tgccgatttg agttcgcgac
4381 actatgtact gcgtttttca ttcttgtatt tgactattta atcctttcta cttgtcgcta
4441 aatataattg ttttagtctt atggcatgat gatagcatat gtgttcaggt ttatagctgt
4501 tgtgtttaaa aattgaaaaa agtggaaaac atctttgtac atttaagtct gtattataat
4561 aagcaaaaag attgtgtgta tgtatgttta atataacatg acaggcacta ggacgtctgc
4621 ctttttaagg cagttccgtt aagggttttt gtttttaaac ttttttttgc catccatcct
4681 gtgcaatatg ccgtgtagaa tatttgtctt aaaattcaag gccacaaaaa caatgtttgg
4741 gggaaaaaaa agaaaaaatc atgccagcta atcatgtcaa gttcactgcc tgtcagattg
4801 ttgatatata ccttctgtaa ataacttttt ttgagaagga aataaaatca gctggaactg
4861 aaccctaaa
```

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-10 provide plots, schematics, and images showing the diet-only mouse model of liver adaptations to chronic metabolic stress. FIG. 1A provides a study design schematic. The Profiling and Analysis includes the following: live sc-RNA-seq, frozen snRNA-seq, and frozen snATAC-seq at 6 months (control diet (CD) and high fat diet (HFD)), 12 months (control diet and high fat diet), and 15 months (control diet and high fat diet), as well as frozen snRNA-seq at 15 months (tumor). FIG. 1B provides a plot of mouse body weight. FIG. 1C provides a plot of Mouse blood albumin concentrations. FIG. 1D provides histological readouts of tissue morphology. FIG. 1E provides histological readouts of fibrosis. FIG. 1F provides a plot of lipid accumulation, and FIGS. 1G-1H provide readouts of hepatocyte ballooning via CK8/18 staining. FIG. 1I provides a plot showing correspondence of CXCR6+CD8+ T cell markers between human MASLD patients and the mouse model. FIG. 1J provides a plot showing annotated T cell subcluster markers. On the x-axis (left to right): Cd8a, Cd4, Pdcd1, Tox, Tigit, Eomes, Ccf7, Sell, Gzmm, Icos, Top2a, Mki67, Cd40Ig, Tnfsf14. FIG. 1K provides T cell subcluster composition with diet. FIGS. 1L-1N provides immunofluorescence images of Tox+ and Cxcr6+ T cells in mouse liver tissue (6mo HFD); FIG. 1L provides an immunofluorescence image with DAPI. FIG. 1M provides an immunofluorescence image with CD4/CD8/Tox. FIG. 1N provides an immunofluorescence image with CD4/CD8/CXCR6. FIG. 1O provides a series of plots showing the quantification of in situ T cell enrichment based on Tox status (6mo HFD, this study). All p-values calculated with Mann-Whitney U test.

FIGS. 2A-20 provide schematics, plots, and images showing the dynamic adaptations of hepatocytes undergoing chronic metabolic stress. FIG. 2A provides an analysis schematic. FIG. 2B provides a plot showing the Longitudinal Increase program, with aggregate expression. FIG. 2C provides a plot showing the expression changes of representative genes. On the x-axis (left to right): lgfbp1, Bcl211, Jun, Klf6, Tcf4, Cdkn1a, Hmgcr, Ldlr, Lcn2, Tm4sf4. FIG. 2D provides a plot showing the enriched GO:BP genesets. FIGS. 2E-2G provide plots showing the Sustained Upregulation program, following the format of FIGS. 2B-2D. On the x-axis of FIG. 2F (left to right): Cd74, Cd1d1, Cd59a, HmgCs1, Cpt1a, Hexa, Smpd2, Pde4d, Mtor, Gpat4. FIGS. 2H-2J provide plots showing the Longitudinal Decrease program, following the format of FIGS. 2B-2D. FIGS. 2K-2M provide plots showing the Sustained Downregulation program, following the format of FIGS. 2B-2D. On the x-axis of FIG. 2I (left to right): Hnf4a, Acaa1a, Aldh5a1, Pank1, Acsl1, Hmgcs2, Nlrp12, Dapk1, Prlr, Nr1 h4. On the x-axis of FIG. 2L: C8a, Fgg, F10, Cps1, Abcb11, Lsr, Esr1, Lifr, Pdia5, Atxn1. FIG. 2N provides an image showing HNF4A protein nuclear abundance log 2 (fold-change) in mouse hepatocytes with chronic metabolic stress, quantified through in situ tissue multiplexed immunofluorescence (N=18 mice, 3 per age (6 months, 12 months, 15 months)×diet condition; n=350,842 nuclei). FIG. 2O provides representative images of HNF4A protein nuclear abundance across chronic metabolic stress progression in mouse liver tissue. GSEA statistical testing through fgsea package; Mann-Whitney U-test used for all other tests. Benjamini-Hochberg correction applied for multiple testing.

FIGS. 3A-3M provide images, plots, and graphs showing that chronic stress adaptations extend to human cohorts and connect to cancer phenotypes and outcomes. FIG. 3A provides MRI (top) and gross imaging (bottom) of mouse model spontaneous hepatocellular carcinoma (HCC). Dashed line indicates tumor; dotted indicates adjacent normal. FIG. 3B provides an H&E-stained image of mouse model spontaneous HCC. FIG. 3C provides a plot showing HCC marker expression in mouse model spontaneous tumors. The markers at the left include: March1, lgfbp1, Fgfr2, Lect2, Krt8, Bcl211, Lcn2, Igf2r, Tbx3, Ctnnb1, Spp1, Axin2, Lgr5, Gpc3, Afp, Mki67, Jag1. FIG. 3D provides a plot showing the Longitudinal Increase program expression in mouse snRNA-seq tumor cells and adjacent normal hepatocytes. FIG. 3E provides a plot showing human bulk liver RNA-seq. FIG. 3F provides a plot showing human snRNA-seq hepatocytes and tumor cells. FIG. 3G provides a plot showing human bulk liver proteomics. FIG. 3H provides a plot showing human HCC survival outcomes. FIGS. 31-3M provide plots showing Longitudinal Decrease program expression, following the format of FIGS. 3D-3H. Survival outcome p-values calculated with log-rank test; all other p-values calculated using Mann-Whitney U test with Benjamini-Hochberg correction.

FIGS. 4A-4N provide plots and graphs showing that chronic metabolic stress induces cancer signatures at early stages of MASLD progression. FIG. 4A provides a plot showing development-associated program expression in mouse snRNA-seq tumor cells and adjacent normal hepatocytes. FIG. 4B provides a plot showing human snRNA-seq hepatocytes and tumor cells. FIG. 4C provides a plot showing mouse snRNA-seq hepatocytes. FIG. 4D provides a plot showing human bulk liver RNA-seq. FIG. 4E provides a plot showing human bulk liver proteomics. FIGS. 4F-4J provide plots showing Human HCC S1 WNT Activation program expression, following the format of FIGS. 4A-4E. FIG. 4K provides a plot showing mouse hepatocyte zonation scoring and annotation. FIG. 4L provides a plot showing HCC S1 WNT Activation program expression by hepatocyte zonation and diet. FIG. 4M provides a plot showing endothelial Rspo3 expression by zonation and diet. FIG. 4N provides a plot showing Tumor-vs-healthy expression differences of this work's hepatocyte adaptation programs, split by tumor etiology. All p-values calculated using Mann-Whitney U test with Benjamini-Hochberg correction.

FIGS. 5A-5F provide plots and schematics showing that chronic metabolic stress drives epigenetic trajectories and WNT pathway priming. FIG. 5A provides a visualization showing chromatin accessibility deviation of TF motifs across age and diet conditions. FIG. 5B provides a visualization showing epigenetic gene activity trajectories of HFD hepatocytes ordered by pseudotime progression. FIG. 5C provides a coverage plot of epigenetic accessibility at Axin2 gene locus across age and diet. FIG. 5D provides a plot showing Accessibility changes of gene-linked peaks at 6-month timepoint, between actual observed HFD-vs-CD fold-changes (red) and GC-and-background-matched null distribution (gray). FIG. 5E provides a plot showing expression changes of genes at 6-month HFD-vs-CD (red) or 15-month tumor-vs-adjacent-normal (brown) comparisons. FIG. 5F provides a computationally-inferred network of TFs predicted to regulate MASLD-relevant gene programs. All p-values calculated using Mann-Whitney U test with Benjamini-Hochberg correction; effect size quantified through Cohen's D.

FIGS. 6A-6M provide plots, graphs, schematics, and images showing human in vitro validation of RELB and SOX4 as regulators of hepatocyte metabolic (mal) adaptation. FIG. 6A provides an experimental design schematic. FIG. 6B provides pseudobulked TF expression profiles. Where applicable, “0.1” and “0.2” indicate isoforms of the same TF gene. At the left: HLF.2, MAFF.1, RELB.2, RORC.2, RELB. 1, RORC.1, SP11.1, None. Lipid, BFP.Lipid, None. Control, BFP.Control, MAFF.2, HLF.1, SP11.2, KLF.1, IRF2.1, JUND.1, DBP.1, SOX4.1, ONECUT1.1, CUX2.1, RXRA.1, JUN.1, PPARA.1. FIG. 6C provides a plot showing RELB's effect sizes on gene program expression. Radius equals |Cohen's D| if concordant directionality with human MASLD/HCC, and 0 if discordant. FIG. 6D provides a plot showing RELB expression across human MASLD progression. FIG. 6E provides a plot showing HCC human survival stratified RELB by expression. FIGS. 6F-6H provide plots showing Transcriptomic regulatory effects of SOX4, following format of FIGS. 6C-6E. FIGS. 6I-6L provide plots showing lipid accumulation (I; BODIPY 493), ROS accumulation (J; CellROX), nuclear p53 (K), and nuclear KI67 (L). FIG. 6M provides representative microscopy images supporting FIGS. 61-6L; scale bar 100 μm. Survival outcome p-values calculated with log-rank test; all other p-values calculated using Mann-Whitney U test with Benjamini-Hochberg correction.

FIGS. 7A-7J provide images, plots, and maps showing in vivo validation of HMGCS2 as a regulator of hepatocyte metabolic (mal) adaptation. FIG. 7A provides H&E staining images of wildtype (WT) or Hmgcs2flox/flox; Alb-Cre (HMGCS2 HepKO) mice on CD or HFD. FIG. 7B provides images showing HMGCS2 immunohistochemistry. FIG. 7C provides a plot showing circulating ketone body concentrations after 24-hour fast. FIGS. 7D-7E provide plots showing circulating glucose concentrations (FIG. 7D) or body weights (FIG. 7E). FIG. 7F shows a series of plots showing HMGCS2 HepKO and diet-induced compositional shifts, using Milo-derived neighborhoods contrasting (HFD.HepKO-CD.HepKO)-(HFD.WT-CD.WT). FIG. 7G provides reference mapping of hepatocytes from HMGCS2 HepKO cohort mice to this study's natural stress adaptation progression. FIG. 7H provides gene program expression with diet and HMGCS2 genotype. FIG. 7I provides expression of Hmgcs1 with diet and HMGCS2 genotype. FIG. 7J provides gene expression across diet and HMGCS2. On x-axis of FIG. 7J (left to right): Hmgcs1, Srebf2, Hmgc1, Mvk, Fdps, Fdft1, Sq1e, Sc5d (Cholesterol synthesis); Cpt1a, Acaa1b, Cd36, Plin2, Cidec (Lipid processing/oxidation); Spp1, Lcn2, Insig2, Rtn4, Nrg1, Lgals1, Erbb4, Cdkn1a (HCC Markers); Afp, Krt8, Axin2, Hes1, Igfbp3, Cd24a, Prom1, Jag1, Sox9, Sox4 (Development/Fetal Hepatocyte); Hnf4a, Cps1, Pck1, Hamp, C4a, Pdia5 (Other Hepatocyte Processes).

FIGS. 8A-8H provides plots and graphs showing the characterization of mouse functional, organ-level, and tissue-level phenotypes induced by metabolic stress. FIGS. 8A-8D provide plots showing circulating alanine transaminase concentrations (FIG. 8A), circulating cholesterol (FIG. 8B), histologic fibrosis stage (FIG. 8C), and histologic inflammation score (FIG. 8D) across chronic metabolic stress progression; each dot is an independent mouse. FIGS. 8E-8G provide plots showing intraperitoneal glucose tolerance test across diet conditions at 6-month (FIG. 8E), 12-month (FIG. 8F), and 15-month (FIG. 8G) timepoints (N=3-5 per agexdiet condition); bars indicate standard error of the mean. In FIGS. 8E, F, G: Top trace=High fat diet condition; Bottom trace: Control diet condition. FIG. 8H provides a plot showing tumor occurrence counts in orthogonal mouse validation cohort of spontaneous HCC formation, at an independent mouse facility.

FIGS. 9A-9L provide plots and visualizations showing a data overview and QC metrics of live-tissue scRNA-seq. FIG. 9A provides UMAP visualization of live-tissue scRNA-seq data. FIG. 9B provides a marker gene dotplot visualization and FIG. 9C provides a plot showing the compositional abundance of cell types. In FIG. 9B, the marker genes are Cfi, C8b, Ptprb, Aqp1, Cd79b, Iglc2, Cx3cr1, Thbs1, Itgae, Xcr1, Marco, Vsig4, S100a8, S100a9, Siglech, Ccr9, Cc15, Thy 1, Top2a, Mki67. FIGS. 9D-9F provide plots showing the number of detected UMIs split by mouse (FIG. 9D), age and diet condition (FIG. 9E), or cell type (FIG. 9F). FIGS. 9G-9I provide plots showing the number of detected genes split by mouse (FIG. 9G), age and diet condition (FIG. 9H), or cell type (FIG. 9I). FIGS. 9J-9L provide plots showing the percent of mitochondrial UMIs split by mouse (FIG. 9J), age and diet condition (FIG. 9K), or cell type (FIG. 9L).

FIGS. 10A-10L provide plots and visualizations showing a data overview and QC metrics of frozen-tissue snRNA-seq. FIG. 10A provides a UMAP visualization of frozen-tissue snRNA-seq data. FIG. 10B provides a marker gene dotplot visualization and FIG. 10C provides a plot showing the compositional abundance of cell types. In FIG. 10B, the marker genes are Abcc2, Cesb3, Nrxn1, Reln, Thsd4, Pdgfd, Ptprb, Stab1, Bank1, Ighm, Lyz2, Ccr2, Clec4f, Cd51, S100a9, Csf3r, Siglech, Irf8, Lck, Cd247, Top2a, Mki67. FIGS. 10D-10F provide a series of plots showing the number of detected UMIs split by mouse (FIG. 10D), age and diet condition (FIG. 10E), or cell type (FIG. 10F). FIGS. 10G-10I provide plots showing the number of detected genes split by mouse (FIG. 10G), age and diet condition (FIG. 10H), or cell type (FIG. 10I). FIGS. 10J-10L provide plots showing the percent of mitochondrial UMIs split by mouse (FIG. 10J), age and diet condition (FIG. 10K), or cell type (FIG. 10L). In FIG. 10L, from left to right: Cholangiocyte, CyclingImmune, Endothelial, Hepatocyte, Kupffer, Macrophage, Neutrophil, pDC, Stellate, T cell.

FIGS. 11A-11I provide plots and visualizations showing a data overview and QC metrics of frozen-tissue snATAC-seq. FIG. 11A provides UMAP visualization of frozen-tissue snATAC-seq data. FIG. 11B provides a plot showing chromatin accessibility coverage plate visualization and FIG. 11C provides a plot showing the compositional abundance of cell types. FIGS. 11D-11F show the number of detected fragment counts split by mouse (FIG. 11D), age and diet condition (FIG. 11E), or cell type (FIG. 11F). FIGS. 11G-11I provide plots showing the number of detected peaks split by mouse (FIG. 11G), age and diet condition (FIG. 11H), or cell type (FIG. 11I).

FIGS. 12A-12K provide plots and images showing that mouse macrophage subcluster exhibits transcriptional similarities to human SAMac cells. FIG. 12A provides a dotplot of marker genes distinguishing this study's macrophage subclusters. In FIG. 12A, the marker genes include (left to right) Clqa, Trem2, Cd9, F13a1, Chi313, Cd209a, Clec10a, Ccna2, Cdk1, Cxcl14, Cd300e. FIG. 12B provides a plot showing the module score of SAMac2 markers, split by macrophage subcluster. FIG. 12C provides a dotplot of SAMac marker genes highly expressed in this study's SAMac-like subcluster. In FIG. 12C, the marker genes include (left to right) Clqb, Clqa, Apoe, Clqc, Ctsd, Trem2, Cd63, Dab2, Itm2b, Ctsc, Cd74, Apoc1. FIG. 12D provides a plot showing compositional enrichment of SAMac-like macrophages with high fat diet even at initial timepoint. FIGS. 12E-12F provide images showing HepG2 lipid accumulation (BODIPY 493) and ROS accumulation (CellROX) in control media (FIG. 12E) or lipid media (FIG. 12F); scale bar 100 μm. FIGS. 12G-12H provide plots showing the Sustained Upregulation program (FIG. 12G) and the Sustained Downregulation program (FIG. 12H) expression in HepG2 cells (n=891 cells). FIGS. 12I-12K provide plots showing HepG2 lipid accumulation (FIG. 12I; microscopy), ROS accumulation (FIG. 12J; microscopy), albumin secretion (FIG. 12K; ELISA). Dots indicate independent wells; N=10 wells/condition.

FIGS. 13A-13L provide plots and visualizations showing a data overview an dQC metrics of frozen-tissue tumor snRNA-seq. FIG. 13A provides a UMAP visualization of frozen-tissue tumor snRNA-seq data. FIG. 13B provides a marker gene dotplot visualization and FIG. 13C provides a plot showing the compositional abundance of cell types. In FIG. 13B, the markers include (left to right) Lect2, Igfbp1, Abcc2, Ces3b, Nrxn1, Reln, Thsd4, Pdgfd, Ptprb, Stab1, Bank1, Ighm, Lyz2, Ccf2, Clec4f, Cd51, Lck, Cd247, Top2a. In FIG. 13C, the cell types from top to bottom include Tumor, Heptocyte, Stellate, Cholangiocyte, Endothelial, B cell, Macrophage, Kupffer, T cell. FIGS. 13D-13F provide plots showing the number of detected UMIs split by mouse (FIG. 13D), diet and tumor status (FIG. 13E), or cell type (FIG. 13F). FIGS. 13G-13I show plots showing the number of detected genes split by mouse (FIG. 13G), diet and tumor status (FIG. 13H), or cell type (FIG. 13I). FIGS. 13J-13L provide a series of plots showing the percent of mitochondrial UMIs split by mouse (FIG. 13J), diet and tumor status (FIG. 13K), or cell type (FIG. 13L). Along the x-axes of FIGS. 13D, 13G and 13J, from left to right: CD_Mouse1_Normal; CD_Mouse2_Normal; CD_Mouse3_Normal; HF_Mouse1_Normal; HF_Mouse1_Tumor; HF_Mouse2_Normal1; HF_Mouse2_Normal2; HF_Mouse2_Tumor1; HF_Mouse2_Tumor2. Along the x-axes of FIGS. 13E, 13H, and 13K, from left to right: CD Normal; HFD Adjacent Normal; HFD Tumor. (CD: Control diet; HFD: High Fat Diet). Along the x-axes of FIGS. 13F, 13I, and 13L: B cell, Cholangiocyte, Endothelial, Hepatocyte, Kupffer, Macrophage, Stellate, T cell, Tumor.

FIGS. 14A-14Q provide graphs and plots showing metabolic stress adaptation programs across species, cohorts, measurements, and clinically-relevant outcomes. FIG. 14A provides a gene set enrichment analysis of HCC signatures among differentially-expressed genes between mouse high fat diet tumor and adjacent normal samples. FIGS. 14B-14E provide plots showing the expression in external human MASLD cohort (liver bulk microarray) of the Longitudinal Increase program (FIG. 14B), the Longitudinal Decrease program (FIG. 14C), the development-associated hepatocyte program (FIG. 14D), or the Human HCC S1: WNT Activation program (FIG. 14E). FIG. 14F provides a plot showing the expression of the Sustained Upregulation program in tumor cells and hepatocytes from adjacent normal tissue (15-month HFD). FIG. 14G provides a plot showing the Expression of Sustained Upregulation program in bulk RNA-seq from external human MASLD cohorts. FIG. 14H provides a plot showing the Expression of Sustained Upregulation program in liver microarray from external human MASLD cohort. FIG. 14I provides a plot showing the expression of Sustained Upregulation program in hepatocytes and tumor cells from snRNA-seq from external human MASLD/HCC cohorts. FIG. 14J provides a plot showing protein abundance of the Sustained Upregulation program in proteomics from external human MASLD/cirrhosis cohort. FIG. 14K provides a graph showing the Stratification of TCGA HCC patient survival, based on expression level of Sustained Upregulation program. FIGS. 14L-14Q provide plots showing disease progression, cancer phenotype associations, and the predictive power of the Sustained Downregulation program, following the format of FIGS. 14F-14K. Survival outcome p-values calculated with log-rank test; GSEA net enrichment score and p-value calculated using random permutation testing through fgsea package; all other p-values calculated using Mann-Whitney U test.

FIGS. 15A-15F provide diagrams showing driver genes of overall program scores. Spearman correlation of individual genes with consistent, strong, positive covariance with overall program scores across datasets: Longitudinal Increase program in FIG. 15A, the Longitudinal Decrease program in FIG. 15B, the Sustained Upregulation program in FIG. 15C, the Sustained Downregulation program in FIG. 15D, the Development-Associated Hepatocyte program in FIG. 15E, and the Human HCC S1: WNT Activation program In FIG. 15F.

FIGS. 16A-16J provide plots and visualizations showing the connection of stress adaptation programs to intercellular signaling and acute regeneration. FIGS. 16A-16B provide plots showing the aggregate hepatocyte module score of Sustained Upregulation (in FIG. 16A) or the Sustained Downregulation program expression levels (in FIG. 16B), over diet and inferred spatial zonation. FIG. 16C shows the zonation scoring and binning of endothelial cells from the mouse model. FIGS. 16D-16E show expression of Wnt2 (in FIG. 16D) or Ctnnb1 (in FIG. 16E) in endothelial cells, over diet and inferred spatial zonation. FIG. 16F provides a plot showing the top inferred ligands prioritized as regulating hepatocytes' stress adaptation-associated gene programs. FIG. 16G provides a plot showing the average expression level of inferred ligands across cell types in the live tissue scRNA-seq mouse data. FIG. 16H provides a plot showing the change in ligand expression with metabolic stress across cell types in the live tissue scRNA-seq mouse data. In FIGS. 16F-H, the ligands include Mmp9, P1g, Tgfb1, Jam3, Gdf3, Ctgf, Yars, Rtn4, Nampt, Mlf, Mapt, Ltb, Psen1, Agt, Edn1, Ptgs2, Hgf, Hmgb1, Col4a1, Tfpi, Cd40Ig, App, Angpt13, Inhbc, Serping1, Spp1, Tnfsf11. FIG. 16I provides a plot showing the change in cell type abundances with metabolic stress in the live tissue scRNA-seq mouse data. The cell types at the left of the plot include CD4 T cells, CD8 T cells, B cells, Cycling T cells, Endothelial, Hepatocyte, Kupffer, Macrophage, Neutrophil, NK or GD, pDC, Treg cells. FIG. 16J provides a plot showing the effect size of change in expression levels of this work's stress adaptation programs during acute regeneration (comparing timepoints after acetaminophen overdose to expression at t=0). All p-values calculated using Mann-Whitney U test.

FIGS. 17A-17F provide plots and visualizations showing epigenetic trajectories of hepatocytes adapting to chronic metabolic stress. FIG. 17A provides plots showing the association between transcriptomic expression (x-axis) and chromatin accessibility (y-axis) of gene programs uncovered in this work (pseudobulked tandem snRNA-seq and snATAC-seq datasets). FIGS. 17B-17C provide plots showing low-dimensional embedding and visualization of high-fat diet snATAC-seq hepatocytes, colored by timepoint (in FIG. 17B) or position (in FIG. 17C) along inferred pseudotime progression. FIGS. 17D-17E provide plots showing low-dimensional embedding and visualization of control diet snATAC-seq hepatocytes, colored by timepoint (in FIG. 17D) or position (in FIG. 17E) along inferred pseudotime progression. FIG. 17F provides a heatmap of chromatin accessibility-based gene activity scores in control diet hepatocyte pseudotime progression; rows have matched order with FIG. 5B.

FIGS. 18A-18O provide plots, graphs, and visualizations showing that MATCHA enables prioritization of TFs regulating arbitrary user-defined gene programs and specific cellular phenotypes. FIG. 18A provides a plot showing the Spearman correlation between transcriptomic expression of the ER stress response gene program (defined externally to this study through the public GO:BP database) and Xbp1 motif accessibility within program-co-accessible linked peaks (this study's natural progression tandem snRNA-seq and snATAC-seq datasets). FIG. 18B provides a plot showing the Spearman correlation between transcriptomic expression of the ER stress response gene program and Xbp1 transcriptomic expression (this study's natural progression snRNA-seq datasets). FIG. 18C provides a plot showing the Spearman correlation between transcriptomic expression of the ER stress response gene program and XBP1 transcriptomic expression (Govaere et al.'s human patient cohort). FIG. 18D provides a graph showing the distribution of Spearman correlations between ER stress response gene program transcriptomic expression and accessibility of all TF motifs at program-coaccessible linked peaks (this study). FIG. 18E provides a graph showing the distribution of Spearman correlations between ER stress response gene program transcriptomic expression and expression of all TFs (this study). FIG. 18F provides a graph showing the distribution of Spearman correlations between ER stress response gene program transcriptomic expression and expression of all TFs (Govaere et al., the disclosure of which is incorporated herein by reference in its entirety for all purposes). FIG. 18G provides a plot showing maximally prioritized TFs inferred as driving (top 5) or repressing (bottom 5) ER stress response gene program. FIG. 18H provides a plot showing the Spearman correlation between transcriptomic expression of the beta-oxidation response gene program (defined externally to this study through the public GO:BP database) and PPARA::RXRA motif accessibility within program-coaccessible linked peaks (this study's natural progression tandem snRNA-seq and snATAC-seq datasets). FIG. 18I provides a plot showing the Spearman correlation between transcriptomic expression of the beta-oxidation gene program and Ppara transcriptomic expression (this study's natural progression snRNA-seq datasets). FIG. 18J provides a plot showing the Spearman correlation between transcriptomic expression of the beta-oxidation gene program and PPARA transcriptomic expression (Govaere et al.'s human patient cohort). FIG. 18K provides a graph showing the distribution of Spearman correlations between beta-oxidation gene program transcriptomic expression and accessibility of all TF motifs at program-coaccessible linked peaks (this study). FIG. 18L provides a graph showing the distribution of Spearman correlations between beta-oxidation gene program transcriptomic expression and expression of all TFs (this study). FIG. 18M provides a graph showing the distribution of Spearman correlations between beta-oxidation gene program transcriptomic expression and expression of all TFs (Govaere et al). FIG. 18N provides a plot showing the maximally prioritized TFs inferred as driving (top 5) or repressing (bottom 5) beta-oxidation gene program. FIG. 18O provides a plot showing a summary of inferred regulatory relationships between TFs chosen for experimental validation and each stress adaptation-associated gene program. Shown along the x-axis are RELB, SOX4, CUX2, DBP, HLF, IRF2, JUN, JUND, KLF4, MAFF, ONECUT1, RORC, SPI1, PPARA::RXRA, NR1H2::RXRA, NR1H4::RXRA, NR4A2::RXRA, RARA::RXRA, RXRA::VDR.

FIGS. 19A-19F provide plots and visualizations showing data overview and QC metrics of arrayed human genetic perturbation (lentiviral TF overexpression) scRNA-seq. FIG. 19A provides a UMAP visualization of arrayed lentiviral TF overexpression scRNA-seq data. FIG. 19B provides a plot showing the number of cells passing QC per overexpressed TF. Provided on the y-axis are RELB.1, MAFF.2, None. Control, IRF2.1, KLF4.1, SOX4.1, DBP.1, RXRA.1, RELB.2, ONECUT1.1, BFP.lipid, RORC.1, None. Lipid, JUND.1, BFP.Control, SPI1.2, MAFF.1, CUX2.1, SPI1.1, RORC.2, HLF.1, HLF.2, JUN.1. FIG. 19C provides a plot showing differentially-expressed genes between PPARA-overexpressing vs. BFP-overexpressing cells in lipid-rich media. FIG. 19D provides a plot showing the number of detected UMIs, split by TF. FIG. 19E provides a plot showing the number of detected genes, split by TF. FIG. 19F provides a plot showing the percent of mitochondrial UMIs, split by TF. The x-axes of FIGS. 19D-F show BFP. Control, BFP.Lipid, CUX2.1, DBP.1, HLF.1, HLF.2, IRF2.1, JUN.1, JUND.1, KLF4.1, MAFF. 1, MAFF.2, None. Control, None.Lipid, ONECUT1.1, PPARA.1, RELB.1, RELB.2, RORC.1, RORC.2, RXRA.1, SOX4.1, SPI1.1, SPI1.2.

FIGS. 20A-20K provide plots, graphs, and visualizations showing contextualization of regulatory effects of RELB and SOX4 on hepatocytes' metabolic adaptation programs. FIG. 20A provides a plot showing the gene expression change induced by RELB overexpression in lipid-rich media (compared to BFP transduction control in lipid-rich media) (top row), or gene expression change with MASLD/cirrhosis/HCC progression in human patients' hepatocytes (Filliol, A. et al., 2022, Nature, 610, 356-365; human snRNA-seq dataset, the disclosure of which is incorporated herein by reference in its entirety for all purposes). snRNA-seq datasets have been deposited in the Gene Expression Omnibus (GEO) database under accession numbers GSE174748, GSE212047, as well as GSE172492, GSE158183, and GSE185477 (Filliol, A. et al., Ibid.). Shown at the top of FIG. 20A are the following: RELB, JUN, KLF6, EPHX1, ALDH1A1, ECH1, DHCR24, FABP1, FGB, SPP1, IL32, LGR5, CD24, CDKN1A, CD74, CD151. LGALS1, SQSTM1. FIGS. 20B-20H provide plots (violin plots) showing TCGA HCC samples stratified by CNV status at RELB gene locus, with expression of RELB itself (FIG. 20B), Development-Associated Hepatocyte gene program (FIG. 20C),Human HCC S1: WNT Activation gene program (FIG. 20D), Sustained Upregulation gene program (FIG. 20E), Longitudinal Increase gene program (FIG. 20F), Sustained Downregulation gene program (FIG. 20G), Longitudinal Decrease gene program (FIG. 20H). FIG. 20I provides a plot showing gene expression change induced by SOX4 overexpression in lipid-rich media (compared to BFP transduction control in lipid-rich media) (top row), or gene expression change with MASLD/cirrhosis/HCC progression in human patients' hepatocytes (Filliol et al., Ibid., human snRNA-seq dataset, the disclosure of which is incorporated herein by reference in its entirety for all purposes). Shown at the top of FIG. 20I are the following: SOX4, NR1H4, PPARA, HNF4A, PHYH, GPD1, AKR1D1, DPYD, EHHADH, SERPINF2, PLG, FABP1, FGG, LGR5, CD24, CDKN1A, NKD1, CD151, MDK, AIFM2, AKR1C3, ROBO1. FIG. 20J provides a graph showing the distribution of expression fold-changes among genes undergoing significant changes with SOX4 overexpression in lipid-rich media (compared to BFP transduction control in lipid-rich media). FIG. 20K provides a plot showing the fold-change of genes involved in lipid handling and oxidative stress response with overexpression of SOX4 or RELB in lipid-rich media (compared to BFP transduction control in lipid-rich media). Shown on x-axis are ABCC1, CPT1A, FABP1, GSTA1, MT1E, MT2A. All p-values calculated using Mann-Whitney U test.

FIGS. 21A-21F provide plots and graphs showing characterization of HMGCS2 with chronic metabolic stress adaptation and hepatocyte-specific knockout cohort. FIG. 21A provides a plot showing Hmgcs2 expression during chronic metabolic stress natural progression in mice (snRNA-seq). FIG. 21B provides a plot showing HMGCS2 expression during MASLD in human patient cohorts (bulk RNA-seq). FIG. 21C provides a plot showing HMGCS2 expression during MASLD/cirrhosis/HCC in human patient cohorts (snRNA-seq). FIG. 21D provides a graph showing stratification of TCGA HCC patient survival, based on expression of HMGCS2. FIG. 21E provides a plot showing ALT levels in HMGCS2 knockout cohort (Kruskal-Wallis p=0.0046). FIG. 21F provides a plot showing cholesterol levels in HMGCS2 knockout cohort (Kruskal-Wallis p=0.0047). Survival outcome p-values calculated with log-rank test. ALT and cholesterol statistics calculated with Kruskal-Wallis test and post-hoc Mann-Whitney U test with Benjamini-Hochberg multiple-testing correction. All other p-values calculated using Mann-Whitney U test.

FIGS. 22A-22L provide plots and visualizations showing a data overview and QC metrics of frozen-tissue snRNA-seq on hepatocyte-specific Hmgcs2 knockout cohort. FIG. 22A provides a UMAP visualization of frozen-tissue tumor snRNA-seq data in hepatocyte-specific Hmgcs2 knockout cohort. FIG. 22B provides a marker gene dotplot visualization and FIG. 22C provides a plot showing the compositional abundance of cell types. In FIG. 22B, the markers include (left to right) Abcc2, Ces3b, Nrxn1, Reln, Thsd4, Pdgfd, Ptprb, Stab1, Bank1, Ighm, Lyz2, Ccr2, Clec4f, Cd51, Lck, Cd247, Top2a. In FIG. 22C, the first bar of each group of bars represents HFD.WT, the second bar represents HFD, LiKO, the third bar represents CD.WT, and the fourth bar represents CD.LiKO. The cell types include Hepatocyte, Stellate, Cholangiocyte, Endothelial, B cell, Macrophage, Kupffer, T cell. FIGS. 22D-22F provide plots showing the number of detected UMIs split by mouse (FIG. 22D), diet and Hmgcs2 genotype status (FIG. 22E), or cell type (FIG. 22F). FIGS. 22G-22I provide a series of plots showing the number of detected genes split by mouse (FIG. 22G), diet (FIG. 22H) and Hmgcs2 genotype status, or cell type (FIG. 22I). FIGS. 22J-22L provide a series of plots showing the percent of mitochondrial UMIs split by mouse (FIG. 22J), diet and tumor status (FIG. 22K), or cell type (FIG. 22L). Survival outcome p-values calculated with log-rank test; all other p-values calculated using Mann-Whitney U test.

FIGS. 23A and 23B provide visualizations of expression of stress adaptation gene programs associated with HCC susceptibility or resistance to chemotherapy/related drugs, which include Temsirolimus, Bosutinib, BEZ235, AT-406, Barasertib, ABT-263, YM155, JNJ-26854165, Epirubicin, Doxorubicin, Topotecan, Panobinostat, Belinostat, Alisertib, Paclitaxel, Vinblastine, Docetaxel, PF-562271, Ganetespib, Trametinib, (+)-JQ1, GSK126, dBET6, A1874, Talazoparib, Dasatinib, Simvastatin, Gemcitabine HCL, Pirarubicin, PI-103, Etoposide, Fluorouracil, Larotrectinib, DZNeP, OSI-906, Erastin, BGJ398, Lenvatinib, Rapamycin, PD173074, Vorasidenib, Piperlongumine, FG-4592, NU-7441, Methotrexate, ARV-771, AT-7519, CP-466722, UNC6852, Olaparib, Afatinib, Cobimetinib, Foretinib, BI2536, I-BET151, GSK1838705A, Ceritinib, Capmatinib, Daporinad, Dovitinib, Sunitinib, Ceralasertib, Crizotinib, Sorafenib, AZD1390, TAE684, 17-AAG, AZD6244, Regorafenib, Tipifarnib, Tivantinib, Ponatinib, AZD7762, Vorinostat, YK-4-279, and Ispinesib. Liver diseases associated with stress are stratified using the markers described herein (e.g., markers showing longitudinal increase, sustained upregulation, etc.) and treated with the corresponding agents delineated in FIGS. 23A and 23B and noted above. The drug target pathways include: 1. IGFR signaling, 2. TOR signaling, 3. ABL signaling, 4. PI3K signaling, 5. MAPK signaling, 6. RTK signaling, 7. FGFR signaling, 8. EGFR signaling, 9. Histone acetylation, 10. Chromatin reader, 11. Histone methylation, 12. Genome integrity, 13. Apoptosis, 14. Mitosis, 15. Cell cycle, 16. DNA replication, 17. Cytoskeleton, 18. Protein folding, 19. Metabolism, 20. Reactive oxygen species, 21. Other.

### DETAILED DESCRIPTION OF THE EMBODIMENTS

Featured and provided herein are compositions and methods for characterizing and treating hepatocellular carcinoma (HCC). The disclosure is based, at least in part, on the discovery of a set of transcriptional (e.g., RELB, Sox4) and metabolic (HMGCS2) mediators that co-regulate and couple tradeoffs between developmental phenotypes, hepatocyte identity, and tissue-level functions.

Under chronic stress, cells must balance competing demands between cellular survival and tissue function. In metabolic dysfunction-associated steatotic liver disease (MASLD, formerly NAFLD/NASH), hepatocytes cooperate with structural and immune cells to perform crucial metabolic, synthetic, and detoxification functions despite nutrient imbalance. While prior work emphasized stress-induced drivers of cell death, longitudinal adaptations and responses of surviving cells remain unclear: which pathways and programs define dynamic cellular responses, what regulatory factors mediate (mal) adaptations, and how cellular adaptations connect to tissue-scale dysfunction and long-term disease outcomes. Here, by applying longitudinal single-cell multi-omics to a mouse model of chronic metabolic stress and extending to human cohorts, it was shown that stress drives survival-linked tradeoffs and metabolic rewiring: shifts towards development-associated states in non-transformed hepatocytes, accompanied by decreases in canonical functions. Diet-induced adaptations occur significantly prior to tumorigenesis but parallel tumorigenesis-induced phenotypes and predict shortened human cancer survival. Through the development of a novel computational gene regulatory inference framework and human in vitro and mouse in vivo genetic perturbations, transcriptional (RELB, SOX4) and metabolic (HMGCS2) mediators were identified that co-regulate and couple tradeoffs between developmental phenotypes, hepatocyte identity, and tissue-level functions. This work defines cellular principles of tissue adaptation to chronic stress, connects environmental stress adaptations to long-term disease outcomes and cancer hallmarks, and unifies diverse axes of cellular dysfunction around core causal factors.

### Hepatocytes and Chronic Stress

These epidemiological and mutation cohort studies raise the hypothesis that, in addition to experiencing mutational accumulation, hepatocytes may respond to chronic stress by developing progressively dysfunctional cell states that are not genetically defined but are poised for tumorigenesis. Work in other organs has described environmental stressors driving non-mutational priming for longer-term dysfunction and tumorigenesis, manifesting as transcriptional and epigenetic adaptations in response to inflammation in the pancreatic epithelium and high-fat diets in intestinal stem cells.

However, prior work in MASLD has largely focused on tissue-level histology or organ-level function in the context of specific gene knockouts or immune subset depletions, or examined broad contributors to cell death (such as reactive oxygen species, unfolded protein response, or lipotoxicity). Comparatively little is known about phenotypic changes in surviving cells and their dynamics. Outstanding questions include: which pathways and functional tradeoffs are induced with progressive exposure to environmental stressors? how do early adaptations connect to long-term consequences like tumor outcomes? and, which decision-making circuits causally mediate cellular (mal) adaptations? Knowledge of the temporal hierarchy of stress adaptations (and accompanying disease repercussions) would help elucidate how tissues coordinate homeostatic functions while buffering stresses affecting constituent cells. Furthermore, the discovery of cell-extrinsic and cell-intrinsic drivers of these processes could lead to novel therapeutic targets and improved patient stratification.

As reported below, the question of how chronic metabolic stress drives functional tradeoffs between cellular identity, homeostatic function, and cancer-associated states was examined. Longitudinal, single-cell, multi-omic analyses of a diet-only mouse model of chronic stress via metabolic overload (spanning early steatosis to spontaneous tumorigenesis) were conducted. With these resources and extensions to human MASLD/HCC cohorts, the progression of hepatocyte adaptations to chronic metabolic stress was defined, including upregulation of early developmental markers, anti-apoptotic/pro-survival effectors, and WNT signaling. These adaptations occur at the expense of core identity and tissue-level functions, such as the expression of lineage-determining transcription factors, rate-limiting enzymes, and immunomodulatory secreted proteins. Through the development of computational frameworks to discover causal regulators driving disease-associated gene programs and experimental genetic perturbations (human in vitro and mouse in vivo), RELB, SOX4, and HMGCS2 were validated as causally driving hepatocyte dysregulation and inducing early shifts towards cancer- and development-associated states. These results define principles of cellular responses to chronic stress in non-transformed tissue and connect them to cancer-associated sequelae, suggesting avenues by which initial stress adaptations perturb cellular states, priming for long-term tissue dysfunction, and disease outcomes.

### Biomarkers

Measurements of expression levels of markers (e.g., polypeptide and/or polynucleotides encoding polypeptides described herein, such as RELB, SOX4, HMGCS2) are used to identify biological samples from subjects having a propensity to develop hepatocellular carcinoma. In particular embodiments, a biomarker is an organic biomolecule that is differentially present in a sample taken from a subject of one phenotypic status (e.g., having a disease, such as hepatocellular carcinoma (HCC)) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease (e.g., having a disease, such as hepatocellular carcinoma (HCC)).

A biomarker described herein may be detected in a biological sample of the subject (e.g., tissue, fluid), including, but not limited to blood, blood serum, plasma, saliva, urine, ascites, cyst fluid, a homogenized tissue sample (e.g., a tissue sample, such as a liver sample, obtained by biopsy), a cell isolated from a patient sample, and the like.

The disclosure provides panels comprising isolated biomarkers. The biomarkers can be isolated from biological fluids. They can be isolated by any method known in the art. In certain embodiments, this isolation is accomplished using the mass and/or binding characteristics of the markers. For example, a sample comprising markers can be subject to chromatographic fractionation and subject to further separation by, e.g., acrylamide gel electrophoresis. Knowledge of the identity of the biomarker also allows their isolation by immunoaffinity chromatography. In some embodiments, biomarkers described herein are fixed to a substrate (e.g., chips, beads, microfluidic platforms, membranes).

TABLE 1A
Selected Markers

| | Longitudinal | Sustained | Sustained |
|---|---|---|---|
| Longitudinal increase | Decrease | Upregulation | Downregulation |
| IGFBP1 | HNF4A | HMGCS1 | C8A |
| BCL2L1 | ACAA1 | CPT1A | FGG |
| JUN | ALDH5A1 | MTOR | F10 |
| KLF6 | PANK1 | GPAT4 | CPS1 |
| TCF4 | ACSL1 | HEXA | ABCB11 |
| CDKN1A | HMGCS2 | PDE4D | ESR1 |
| HMGCR | NLRP12 | SMPD2 | LSR |
| LDLR | DAPK1 | CD74 | LIFR |
| LCN2 | PRLR | CD1D | PDIA5 |
| TM4SF4 | NR1F4 | CD59 | ATXN1 |
| BTG2 | GLYAT | GRPEL2 | ST6GAL1 |
| KRT18 | HIBADH | KLF6 | C8B |
| YWHAZ | PBLD | ARL4A | EGFR |
| ATF3 | DPYS | ANXA5 | ITIH1 |
| JUND | MAOB | ZFAND5 | IL13RA1 |
| MCL1 | SCP2 | GDF15 | SLC30A10 |
| TMEM87B | SLC25A13 | ARPC5 | SERPINF2 |
| SUCO | A1CF | MDM2 | AMFR |
| SLC38A2 | DMGDH | S100A10 | CYP4F14 |
| GDF15 | ALDH8A1 | CSTB | ERP44 |

TABLE 1B

| | | | Other |
|---|---|---|---|
| | | Tested | prioritized |
| Development-Associated | Human HCC S1: | transcription | transcription |
| Hepatocyte | WNT Activation | factors | factors |
| CDKN1A | CD74 | HLF | NFE2L1 |
| KRT8 | ARCP1B | MAFF | AR |
| CD24 | FLNA | RELB | BATF |
| SOX9 | S100A11 | RORC | BATF3 |
| HES1 | PEA15 | SPI1 | ESRRA |
| EPCAM | GNAI2 | KLF4 | NR1I3 |
| IGFBP3 | ATP1B3 | IRF2 | CEPBA |
| JAG1 | ANXA5 | JUND | TEF |
| DDR1 | IER3 | DBP | RFX2 |
| PROM1 | SRGN | SOX4 | CUX1 |
| LGR5 | | ONECUT1 | NR3C2 |
| AFP | | CUX2 | NFYC |
| | | RXRA | |
| | | JUN | |
| | | PPARA | |

TABLE 2A
Secreted Proteins Linked to Transcription Factors for Peripheral Detection

| CUX2 | DBP1 | HLF1 | IRF2 | JUN | JUND | KLF4 | MAFF | ONECUT1 |
|---|---|---|---|---|---|---|---|---|
| IGF2 | SAA4 | PPIG | PLG | SPP1 | NDRG1 | PLG | C6 | DKK1 |
| DKK1 | TFPI | YWHAQ | SERPINE1 | SAA4 | RELN | IGFBP1 | AMBP | GC |
| RELN | SPP1 | CES1 | FGA | IL6ST | FLNB | CP | FTH1 | PLG |
| LOX | CXCL8 | ORM2 | C6 | INHBE | PLIN2 | IGF2 | SERPINA7 | SERPINE2 |
| HP | CES1 | | | | C3 | TTR | INHBE | SPINK1 |
| CTSB | IL32 | | | | | | LGALS3BP | FABP5 |
| APOA5 | F11 | | | | | | FGA | |
| | HPX | | | | | | | |

TABLE 2B

| PPARA | RORC | RXRA | SPI1 | RELB | SOX4 |
|---|---|---|---|---|---|
| FABP1 | LOX | KNG1 | IGF2 | IL32 | DKK1 |
| BMP4 | CP | NRP2 | LOX | SPP1 | CFL1 |
| S100P | PLIN2 | VCAN | GPX3 | TFPI | MDK |
| CAT | CSTB | DST | TTR | CTSH | NKD1 |
| CTSC | APOH | LDLR | SERPINC1 | CFL1 | GDF15 |
| OAS1 | KRT19 | | ENO1 | SAA4 | PSAP |
| | ITIH1 | | PLG | PSAP | |
| | ITIH3 | | | | |

In one embodiment, the markers being tested are: HLF, MAFF, RELB, RORC, SPI1, KLF4, IRF2, JUND, DBP, SOX4, ONECUT1, CUX2, RXRA, JUN, or PPARA. In one embodiment, the markers being tested are: RELB, SOX4, JUND, ONECUT1, MAFF, RORC, DBP, or RXRA. In one embodiment, the markers being tested are: NFE2L1, AR, BATF, BATF3, ESRRA, NR113, CEPBA, TEF, RFX2, CUX1, NR3C2, or NFYC. In one embodiment, the markers being tested are: THRB, PPARA, or NR1H4.

### Detection of Biomarkers

The biomarkers of this disclosure (e.g., RELB, SOX4, HMGCS2) can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the described embodiments include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described below.

Detection by Sequencing and/or Probes

In particular embodiments, the biomarkers are measured by a sequencing- and/or probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109 (4): 1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a biomarker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TAQGOLD™, T7 DNA polymerase, Klenow fragment of E. coli DNA polymerase, and reverse transcriptase. A useful amplification method is polymerase chain reaction (PCR). In particular, the isolated RNA can be subjected to a reverse transcription assay that is coupled with a quantitative polymerase chain reaction (RT-PCR) in order to quantify the expression level of a biomarker.

Detection of the expression level of a biomarker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoechst, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91 (13): 5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of biomarkers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target biomarker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as ³²P, ¹⁴C, ¹²⁵I, ³H, and ¹³¹I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments as described herein.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the biomarkers described herein are measured by immunoassay. An immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

The disclosure embraces traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In embodiments, a sample is analyzed by means of a biochip (also known as a microarray). The polypeptides and nucleic acid molecules described herein are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a biomarker, or a fragment thereof. In embodiments, a protein biochip described herein binds a biomarker present in a sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker as described herein) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects and embodiments as described herein, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions include, as non-limiting examples, temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentrations for the wash steps are less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In an embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the biomarkers described herein are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2): 880-891) or semi-automated format. This can be accomplished, for example, with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the disclosure is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the described embodiments to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS″ analysis to be performed. Tandem mass spectrometry allows multiple reactions to be monitored at the same time.

Capillary infusion may be employed to introduce the biomarker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the embodiments described herein. Fourier-transform ion cyclotron resonance (FTMS) can also be used for some embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the disclosure is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's PROTEINCHIP® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol) methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used. In an embodiment, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Panels

The present disclosure provides panels of capture molecules, each of which binds a biomarker, and the use of such panels for characterizing the propensity of a subject to develop MASLD, hepatocellular carcinoma, or another liver disease. As would be understood, references herein to a biomarker, a panel of biomarkers, or other similar phrase indicates one or more of the biomarkers described herein (e.g., RelB, Sox4, Hmgcs2).

The disclosure further features the use of such panels for characterizing hepatocellular carcinoma. In embodiments, the panels are used in combination with a classifier (e.g., a machine learning classifier, such as MATCHA) to identify a subject having a propensity to develop hepatocellular carcinoma. The panels are advantageously used for guiding selection of a subject for a hepatocellular carcinoma treatment.

### Hardware and Software

The present disclosure also provides a computer system (e.g., capable of executing code associated with MATCHA) useful in analyzing data associated with biomarker expression, patient selection, and related computations (e.g., calculations associated with a machine learning classifier).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the disclosure can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or

DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Therapeutics

The disclosure provides agents that inhibit markers (e.g., RelB, Sox4, HMGCS2) that are increased in subjects having hepatocyte stress, and/or a propensity to develop liver disease (e.g., MALSD and/or hepatocellular carcinoma (HCC)). In some embodiments, an agent useful for treating HCC alters the expression or activity of a gene or polypeptide that is downstream of RelB, Sox4, or HMGCS2.

In one embodiment, the disclosure features a small molecule inhibitor of RelB, a RelB antibody that inhibits its activity, or an inhibitory polynucleotide (e.g., shRNA, siRNA, antisense polynucleotide) that inhibits RelB expression. Agents that inhibit RelB include, but are not limited to, 1,25-Dihydroxyvitamin D3 (Mineva et al., J Cell Physiol. 2009 September; 220 (3): 593-599), Vinorelbine (Wu et al., Front. Mol. Biosci., 14 Jun. 2023).

In another embodiment, the disclosure features an inhibitor of Sox4. Agents that inhibit Sox4 include, but are not limited to, integrin avb6/8-blocking monoclonal antibody (mAb) (See Bagati et al., Cancer Cell 39, 54-67, Jan. 11, 2021), inhibitory polynucleotides that target Sox4 (e.g., siRNA, shRNA, antisense polynucleotide).

In another embodiment, the disclosure features an inhibitor of HMGCS2. Such inhibitors include, for example, momilactone B (Kang et al., Anim Biotechnol. 2017 Jul. 3; 28 (3): 189-197), HMGCS2 inhibitor (CN105055404B).

Agents of the embodiments disclosed herein may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agents of the disclosed embodiments include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition (e.g., hepatocellular carcinoma). The dosage of a nucleobase oligomer as described is likely to depend on such variables as the type and extent of the disorder, the overall health status of the particular patient, the formulation of the compound excipients, and its route of administration.

With respect to a subject having hepatocellular carcinoma, an effective amount of an agent described herein is sufficient to stabilize, slow, or reduce the proliferation of hepatocellular carcinoma or a condition that precedes HCC. Generally, doses of active polynucleotide compositions of the present disclosure would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an agent and/or compositions of the present disclosure.

A variety of administration routes are available. The methods described herein, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Kits

In another aspect, the disclosure provides kits for aiding in patient selection for treatment and/or characterizing hepatocellular carcinoma (e.g., selecting a treatment method for a subject, selection of a subject for a clinical trial, predicting clinical outcome, and the like), which kits are used to detect biomarkers according to the disclosure. In an embodiment, the kit comprises a drug for use in treatment of hepatocellular carcinoma. In some instances, the kit comprises reagents for collecting a sample from a patient and sequencing RNA from the sample (e.g., RNA-seq). In one embodiment, the kit comprises agents that specifically recognize RelB, Sox4, or HMGCS2. In another embodiment, the kit comprises agents for use in detecting the biomarkers identified herein. In related embodiments, the agents are antibodies or probes (e.g., oligonucleotides).

In another embodiment, the kit comprises a solid support, such as a chip, a microtiter plate or a bead or resin having capture reagents attached thereon, wherein the capture reagents bind the biomarkers of the disclosure. In the case of biospecfic capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In some instances, the kit comprises drug sensitivity information for hepatocellular carcinoma. The drug sensitivity data is provided in some embodiments along with instructions for selecting a patient for administration of a drug based upon biomarker expression in the subject. In embodiments, the instructions provide suitable operational parameters in the form of a label or separate insert. For example, the instructions may inform a consumer about how to collect the sample, how to wash the probe, and/or the particular biomarkers to be detected.

In yet another embodiment, the kit can comprise one or more containers with controls (e.g., biomarker samples) to be used as standard(s) for calibration.

The practice of the present embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the aspects and embodiments disclosed herein. Particularly useful techniques for particular embodiments will be discussed in the sections that follow.

The following examples are put forth so as to provide those of ordinary skill in the art with a complete disclosure and description of how to make and use the assay, screening, and therapeutic methods as described, and are not intended to limit the scope of the present disclosure.

### EXAMPLES

### Example 1: Long-Term High Fat Diet in Wild-Type Mice Mimics Aspects of Human MASLD and Spontaneous Tumorigenesis

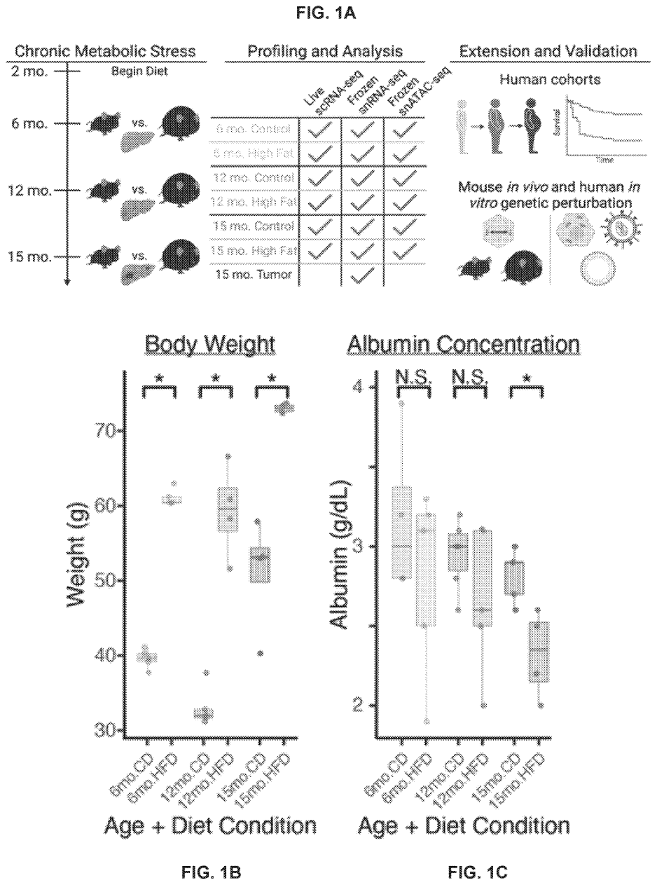

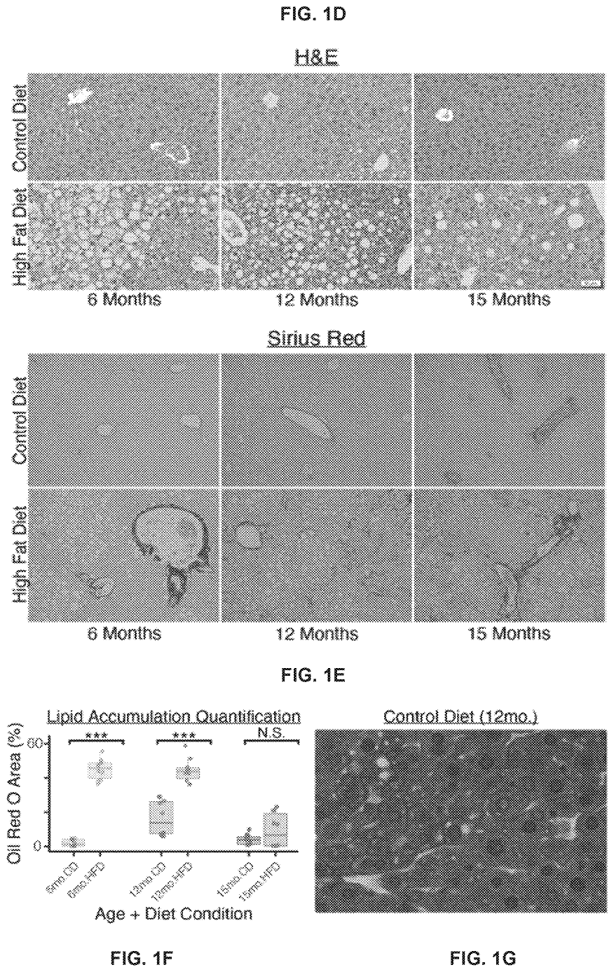

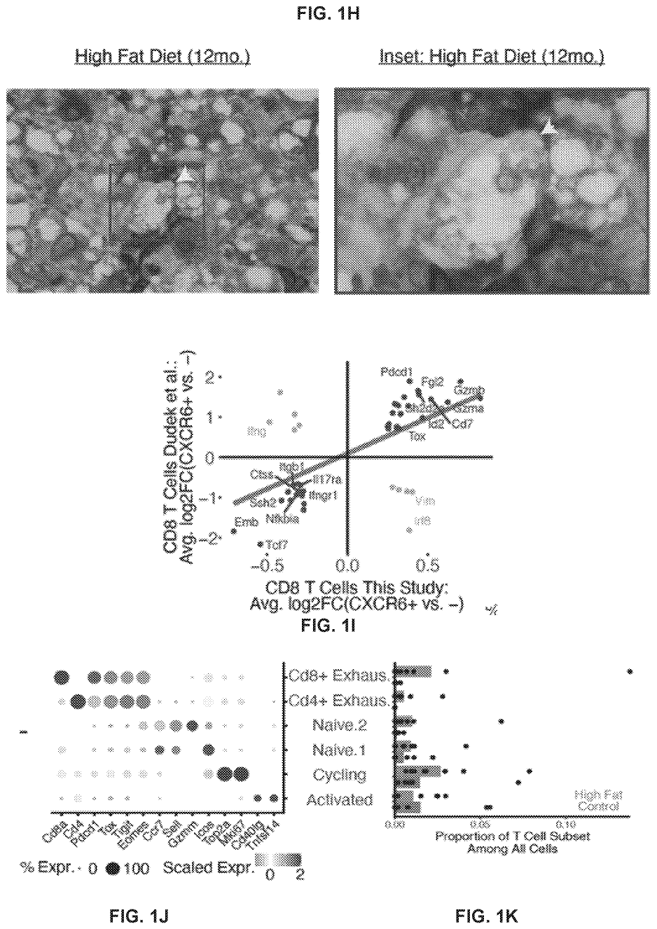

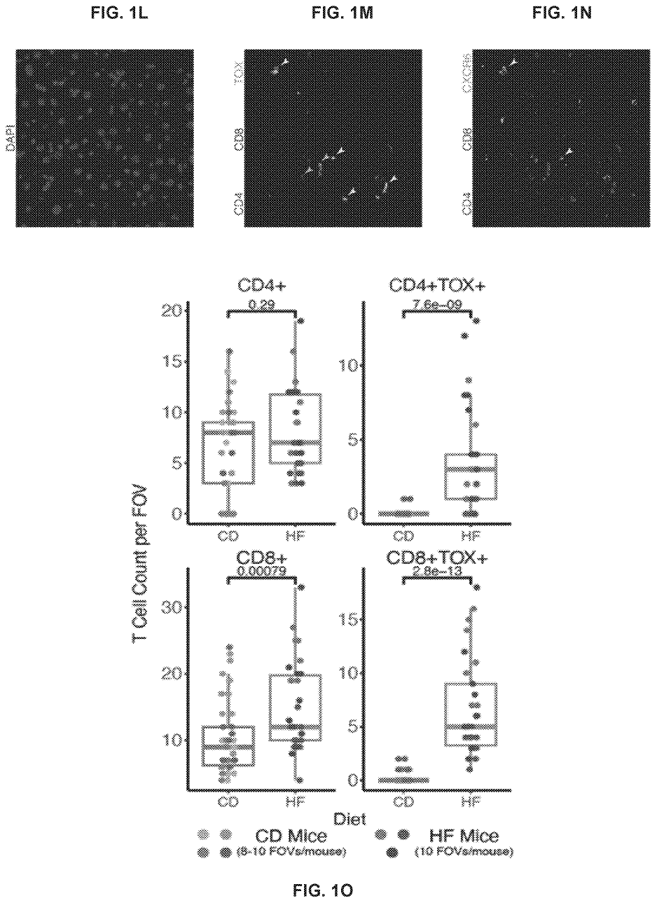

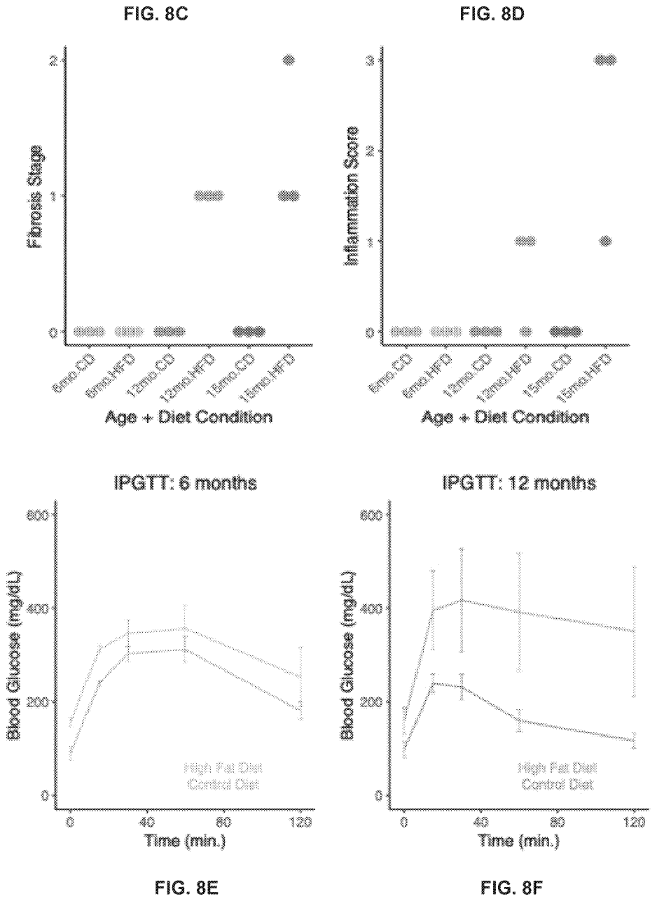

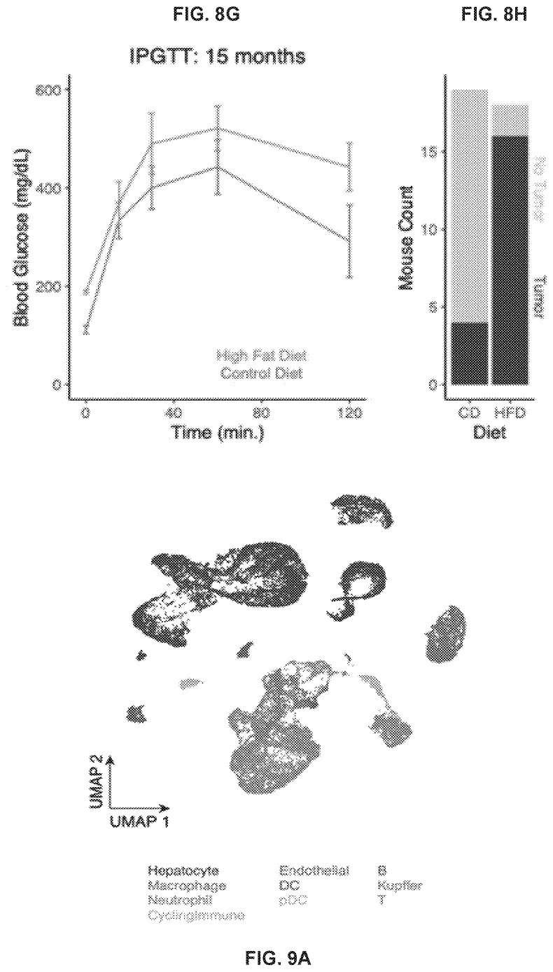

As an exemplar of chronic stress, a high-fat diet (HFD)-mediated liver injury model was studied. In agreement with prior work, it was found that HFD C57BL/6 mice developed obesity, elevated serum cholesterol, increased alanine aminotransferase (ALT) levels, decreased serum albumin, and impaired glucose tolerance, indicative of hepatocellular damage, synthetic dysfunction, and diet-induced systemic insulin resistance (FIG. 1B-C, FIG. 8). Histologically, HFD livers exhibited (initially pericentral) steatosis, lobular inflammation, pericellular “chicken-wire” fibrosis, and hepatocellular ballooning (FIG. 1D-H, FIG. 8). Spontaneous HCC developed without additional genotypic or chemical manipulation, validated in a second mouse cohort at a different institution (16/18 HFD mice vs. 4/19 CD mice developing HCC by 18 months; FIG. 8). These systemic and histologic findings mimic aspects of human MASLD progression: simple steatosis at 6 months, followed by certain features of inflammation, fibrosis, and spontaneous HCC tumorigenesis at 12 and 15 months.

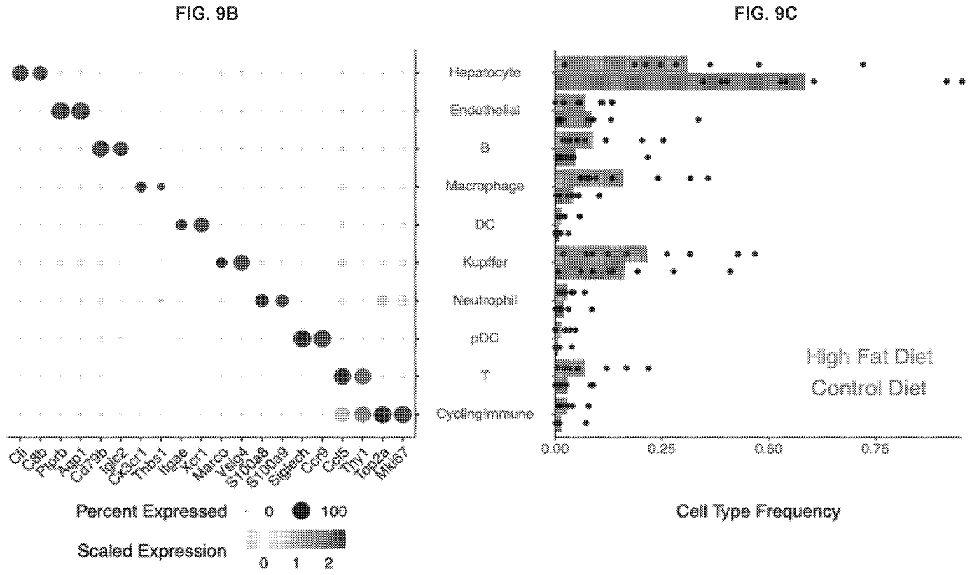

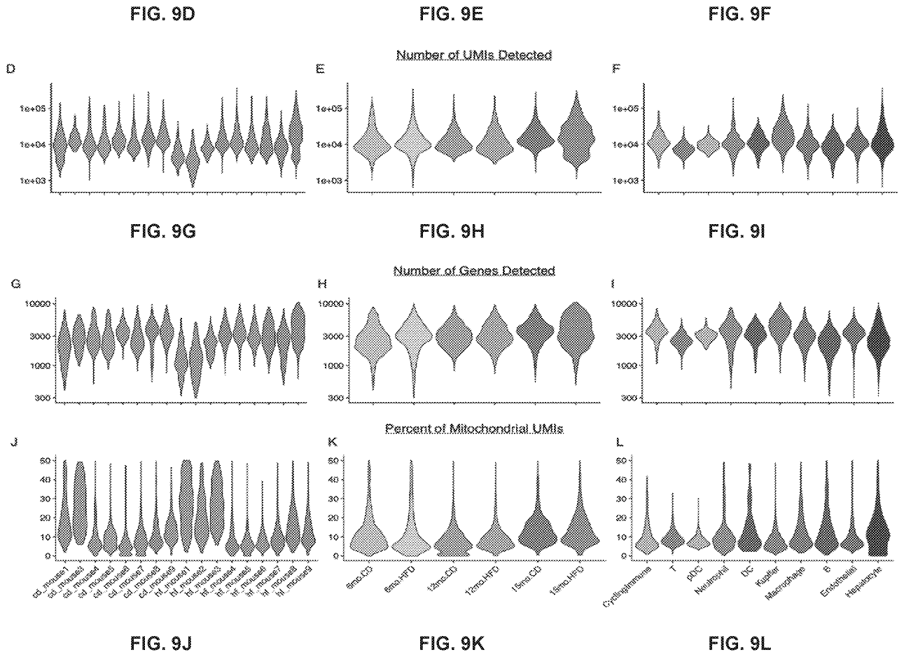

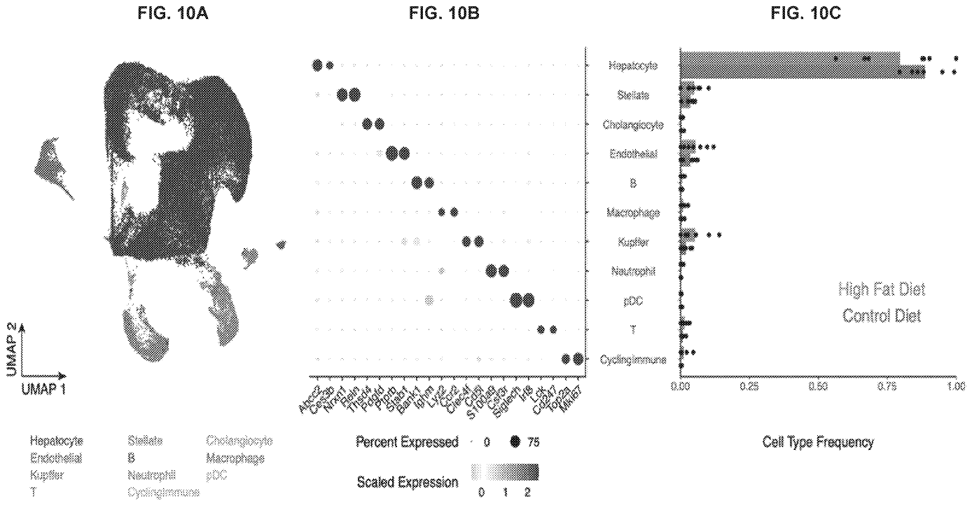

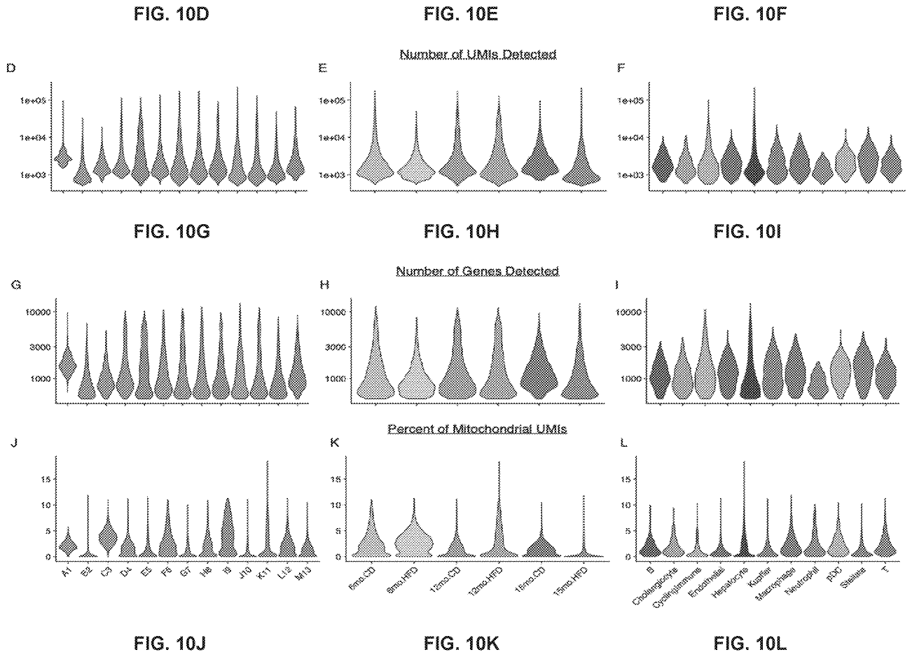

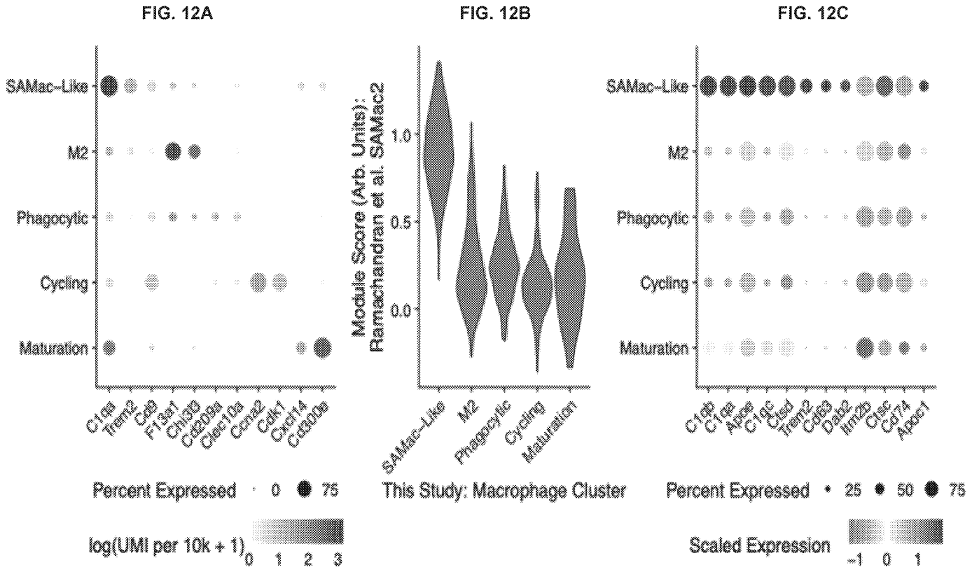

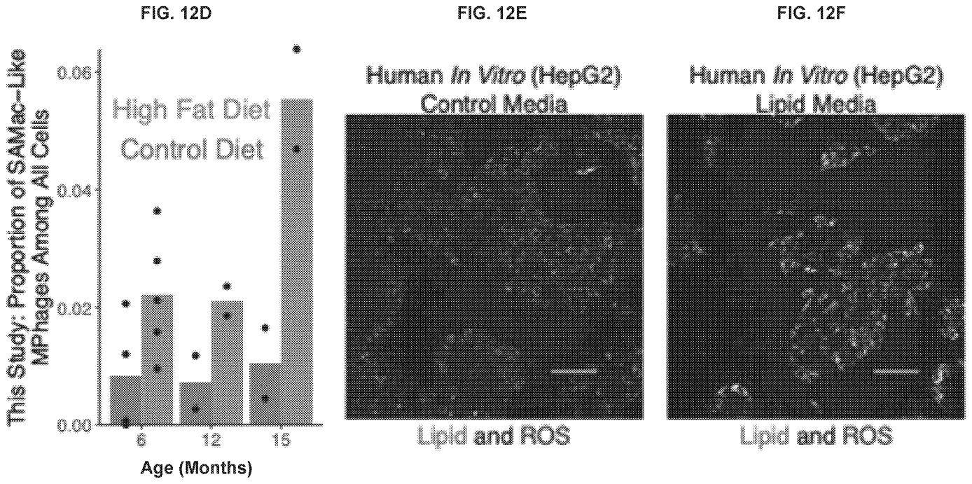

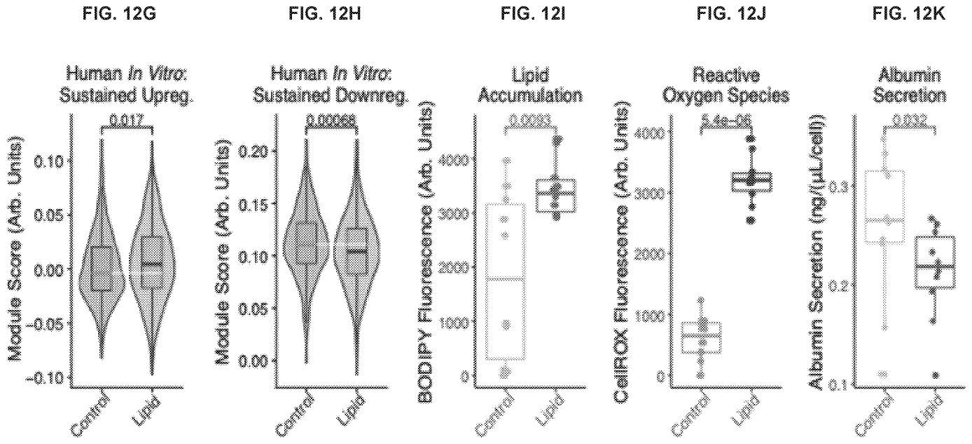

To understand longitudinal adaptations to chronic metabolic stress, immune-biased live tissue scRNA-seq (N=17 mice, n=23,819 cells), epithelial-biased frozen tissue snRNA-seq (N=18 mice, n=79,408 cells), and snATAC-seq (N=13 mice, n=97,113 cells) for HFD and CD mice at each timepoint (FIGS. 9-11) were conducted. Towards human findings on non-parenchymal cells in MASLD, prior work has implicated human CXCR6⁺CD8⁺ T cells (expressing TOX and PDCD1) as drivers of autoaggressive hepatocyte killing in MASLD. Cxcr6⁺Cd8⁺ T cells in the model recapitulated markers of these previously-described human cells, and T cells expressing exhaustion-related markers were compositionally enriched with HFD in the scRNA-seq dataset (FIG. 1I-1K). Multiplexed immunofluorescence on mouse liver revealed that TOX⁺ T cells were strongly enriched in situ as early as 6 months (FIGS. 1L-10). Likewise, prior work identified human scar-associated macrophages (SAMac) localized to cirrhotic regions and capable of activating collagen expression in stellate cells. SAMac markers aligned with a cluster of macrophages in the scRNA-seq data; these SAMac-like mouse macrophages were enriched even at the earliest timepoint before overt fibrosis deposition (FIG. 12A-12D). These results indicate that the mouse model mirrors particular molecular phenotypes of human MASLD and may provide relevant insights into the trajectory of liver dysfunction under chronic metabolic stress.

### Example 2: Metabolic Stress Drives Tradeoffs Between Pro-Survival Pathways and Core Homeostatic Functions

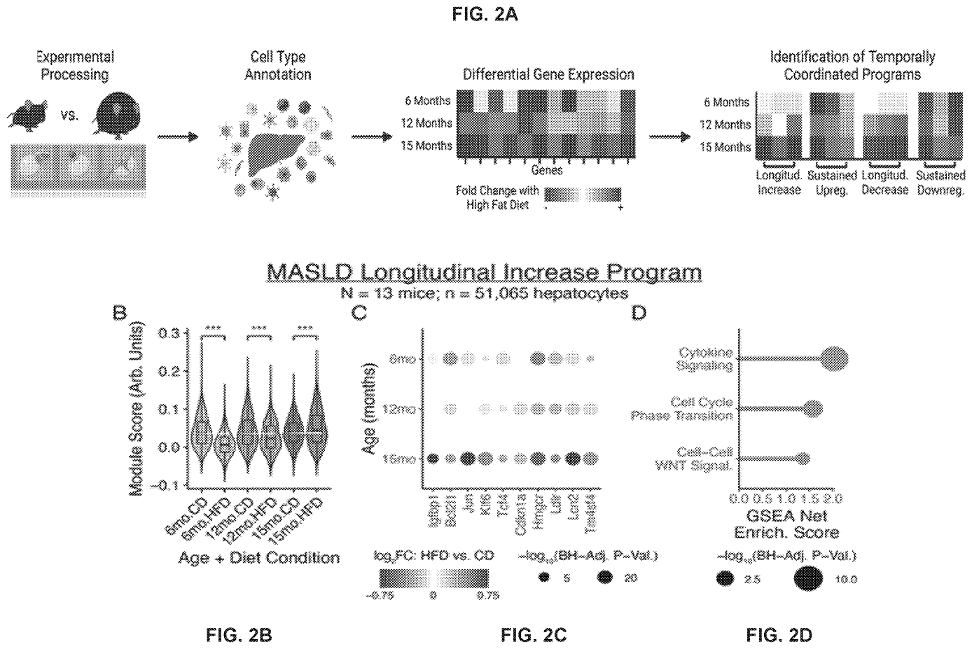

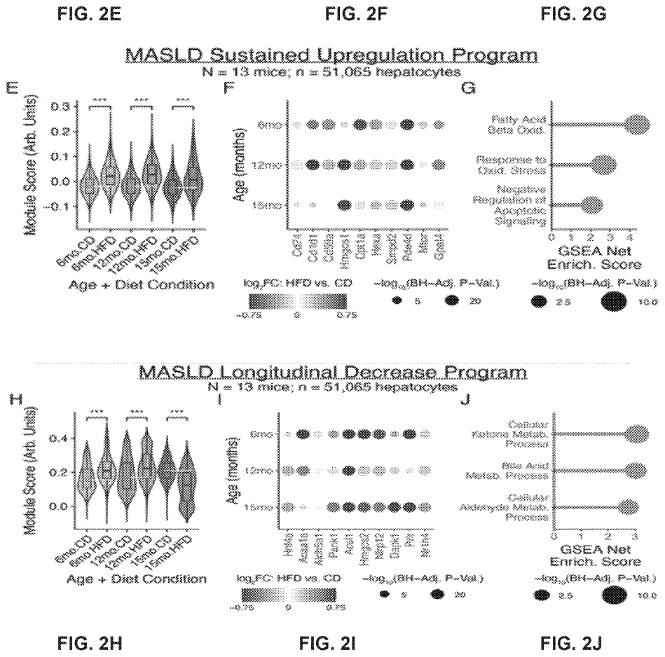

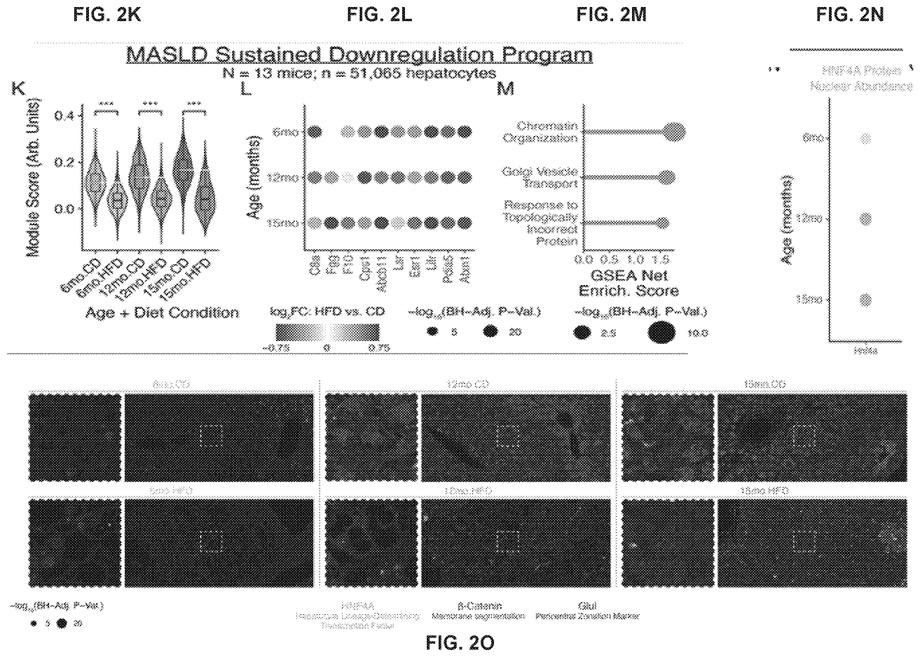

Given the diversity of homeostatic roles performed by hepatocytes, hepatocytes' dynamic stress responses and adaptations were studied. Reasoning that genes with temporally-coordinated expression trajectories may constitute linked biological processes, cellular responses, or regulatory mechanism targets, four expression programs were defined: 1) “Longitudinal Increase” and 2) “Sustained Upregulation”, which capture genes progressively elevated with long-term metabolic stress or consistently elevated, respectively; and 3) “Longitudinal Decrease” and 4) “Sustained Downregulation”, which capture progressive declines or consistent lessening, respectively (FIG. 2A, Table 1).

Examining the Longitudinal Increase program, long-term metabolic stress increased expression of genes involved in pro-survival pathways, WNT signaling, cholesterol metabolism, and intercellular signaling (FIGS. 2B-2D). For instance, Igfbp1, Bcl211, Jun, and Klf6 have well-established roles in promoting hepatocyte survival and inhibiting apoptosis. Increases of key WNT effectors (e.g., Tcf4) and regeneration-elevated regulators of cell cycle and senescence (e.g., Cdknla) suggest connections between stress responses and hepatocyte regeneration. Linking stress responses and altered metabolism, hepatocytes also progressively upregulated the rate-limiting enzyme of cholesterol synthesis (Hmgcr) and key cholesterol uptake regulators (e.g., Ldlr), aligning with increases in cholesterol accumulation in hepatocytes. Towards immune influences on hepatocyte phenotypes, upregulation of inflammation-responsive, regeneration-associated, and HCC-elevated genes like Lcn2 and Tm4sf4 was also observed. As a more unbiased analysis, externally-defined gene sets related to cytokine signaling, cell cycle regulation, and WNT signaling were enriched with the Longitudinal Increase program (FIG. 2D).

The Sustained Upregulation program, capturing aspects of hepatocytes' responses to metabolic stress that are maintained over time, provided further examples of hepatocytes shifting to prioritize pro-survival responses under metabolic stress (FIGS. 2E-2G). Hepatocytes increased expression of receptors mediating immune interactions and exerting either pro-survival or anti-apoptotic effects, including Cd74 (through binding to MIF and promoting hepatocyte survival, in addition to antigen presentation), Cd1d1 (through mitigating iNKT-mediated inflammation and hepatocyte apoptosis), and Cd59a (through preventing formation of complement membrane attack complexes on hepatocytes). Specific cholesterol synthesis and lipid oxidation enzymes were also upregulated, including Hmgcs1 (catalyzing cytoplasmic acetoacetyl-CoA to HMG-CoA) and Cpt1a (rate-limiting enzyme for fatty acid beta-oxidation). Also increased were metabolic regulators including Mtor and Gpat4 and enzymes catalyzing signal transduction metabolites (e.g., Hexa, Smpd2, Pde4d).

However, hepatocytes' pro-survival, WNT-associated, and immune interaction-related responses came at the expense of processes normally associated with hepatocytes' core, homeostatic roles (FIGS. 2H-2J). Hnf4a was progressively downregulated with long-term metabolic stress, particularly notable given its role as a master regulator of hepatocyte identity with wide-ranging effects on hepatocyte functions and fate specification during development. Concordantly, the Longitudinal Decrease program also included peroxisomal oxidation (e.g., Acaa1a), aldehyde processing (e.g., Aldh5a1), CoA biosynthesis (Pank1; rate-limiting), and acyl-CoA processing (e.g., Acsl1). Progressive decreases in Hmgcs2, the rate-limiting enzyme of ketogenesis, were notable given that 1) ketogenesis and cholesterol synthesis compete for the same starting metabolites, and 2) many cholesterol synthesis enzymes (including Hmgcr) followed opposing trajectories. Genes capable of driving cell death and serving as tumor suppressors, like Nlrp12 and Dapk1, were also downregulated. Regulators of broad hepatocyte phenotypes, Prlr and Nr1 h4 (encoding FXR) were likewise reduced with long-term metabolic stress. Prlr serves as a receptor for hormones including prolactin and growth hormone and is upregulated by estrogens, notable given MASLD's dramatically lower incidence in pre-menopause women. The bile acid-activated nuclear receptor FXR has been targeted in clinical trials via agonists including obeticholic acid, but the Phase III REVERSE trial recently failed to reach its primary endpoint, which may indicate opportunities for improved patient stratification based on expression and/or protein abundance of FXR.

Hepatocytes' Sustained Downregulation program also reflected reduction of core hepatocyte and liver functions including secreted complement (C8a; also C6, C8b), coagulation factors (Fgg, F10), urea cycle (Cps1; rate-limiting enzyme), and bile acid regulation (e.g., Abcb11) (FIGS. 2K-M). Lsr regulates circulating triglyceride levels, and its experimental knockout has been shown to increase weight gain, suggesting maladaptive repercussions of its diet-induced downregulation. Downregulation of genes involved in proteostasis (e.g., Pdia5) and chromatin interactions (e.g., Atxn1) was also observed. As complementary links to genes captured in other modules, downregulation of complement protein production harmonizes with Cd59a's role in preventing complement-mediated hepatocyte death (Sustained Upregulation program). Esr1 encodes estrogen receptor alpha, and its downregulation aligns with progressive losses of Prlr (Longitudinal Decrease program). Likewise, Lifr knockout is associated with increases in Lcn2 secretion (Longitudinal Increase program) and elevated tumorigenesis, connecting metabolic stress-induced downregulation of an upstream receptor to subsequent pro-inflammatory, cancer-associated hepatocyte responses.

Towards validating dynamic shifts in hepatocyte phenotypes with chronic stress, HNF4A was analyzed for its regulation of hepatocytes' developmental differentiation and mature function. In situ mouse liver immunofluorescence demonstrated progressive decreases in HNF4A nuclear protein abundance with chronic metabolic stress, aligning with its transcriptional trajectory as part of the Longitudinal Decrease program (FIG. 2N). Human liver HepG2 cells were cultured in lipid-rich media, finding gene expression changes and functional metabolic and synthetic dysregulation concordant with sustained in vivo hepatocyte shifts (FIGS. 12E-12K).

Thus, hepatocytes adapt to chronic metabolic stress by increasing expression of pro-survival responses including anti-apoptotic effectors, WNT signaling, and intercellular interaction mediators. This pro-survival adaptation comes at the expense of genes linked to core hepatocyte identity and homeostatic function, including multiple classes of secreted molecules, transcription factors including HNF4A and FXR, and metabolic enzymes.

TABLE 3
Expression Programs and Related Genes

| Geneset | Gene | Geneset | Gene | Geneset | Gene |
|---|---|---|---|---|---|
| Sustained | S100a10 | Sustained | Slco1a1 | Longitudinal | Lcn2 |
| Upregulation | | Downregulation | | Increase | |
| Sustained | Pde4d | Sustained | Hamp | Longitudinal | Fabp5 |
| Upregulation | | Downregulation | | Increase | |
| Sustained | Enpp2 | Sustained | Lsr | Longitudinal | Serpine2 |
| Upregulation | | Downregulation | | Increase | |
| Sustained | Gdf15 | Sustained | Pdia5 | Longitudinal | Egr1 |
| Upregulation | | Downregulation | | Increase | |
| Sustained | Rgs16 | Sustained | Apom | Longitudinal | Ccnd1 |
| Upregulation | | Downregulation | | Increase | |
| Sustained | Phlda1 | Sustained | Abcb11 | Longitudinal | Igfbp2 |
| Upregulation | | Downregulation | | Increase | |
| Sustained | Anxa5 | Sustained | Bst2 | Longitudinal | Atf3 |
| Upregulation | | Downregulation | | Increase | |
| Sustained | Spc24 | Sustained | Srd5a1 | Longitudinal | Jun |
| Upregulation | | Downregulation | | Increase | |
| Sustained | Gda | Sustained | St3gal1 | Longitudinal | Fos |
| Upregulation | | Downregulation | | Increase | |
| Sustained | Klf6 | Sustained | C8a | Longitudinal | Tff3 |
| Upregulation | | Downregulation | | Increase | |
| Sustained | Arl4a | Sustained | C8b | Longitudinal | Rarres1 |
| Upregulation | | Downregulation | | Increase | |
| Sustained | Cldn2 | Sustained | Efna1 | Longitudinal | Hmgcr |
| Upregulation | | Downregulation | | Increase | |
| Sustained | Cd59a | Sustained | H2-Q7 | Longitudinal | Serpina3c |
| Upregulation | | Downregulation | | Increase | |
| Sustained | Plk3 | Sustained | Cdhr5 | Longitudinal | Serpina3n |
| Upregulation | | Downregulation | | Increase | |
| Sustained | Erbb4 | Sustained | Slc30a10 | Longitudinal | Krt18 |
| Upregulation | | Downregulation | | Increase | |
| Sustained | Atp9a | Sustained | Cps1 | Longitudinal | Sqle |
| Upregulation | | Downregulation | | Increase | |
| Sustained | Hmgcs1 | Sustained | Ambp | Longitudinal | Nnmt |
| Upregulation | | Downregulation | | Increase | |
| Sustained | Prodh | Sustained | Slc3a1 | Longitudinal | Acs14 |
| Upregulation | | Downregulation | | Increase | |
| Sustained | L3hypdh | Sustained | Lrrc3 | Longitudinal | Btg2 |
| Upregulation | | Downregulation | | Increase | |
| Sustained | Gins4 | Sustained | St6gal1 | Longitudinal | Cdkn1a |
| Upregulation | | Downregulation | | Increase | |
| Sustained | Mmd | Sustained | Itih1 | Longitudinal | Pfkfb2 |
| Upregulation | | Downregulation | | Increase | |
| Sustained | G0s2 | Sustained | Fgg | Longitudinal | Sfi1 |
| Upregulation | | Downregulation | | Increase | |
| Sustained | Slc16a12 | Sustained | C6 | Longitudinal | Junb |
| Upregulation | | Downregulation | | Increase | |
| Sustained | H2-Ab1 | Sustained | Serpinf2 | Longitudinal | Arhgef3 |
| Upregulation | | Downregulation | | Increase | |
| Sustained | Cstb | Sustained | Cbs | Longitudinal | Serpina3m |
| Upregulation | | Downregulation | | Increase | |
| Sustained | Cd74 | Sustained | F10 | Longitudinal | Klf6 |
| Upregulation | | Downregulation | | Increase | |
| Sustained | Rsrp1 | Sustained | Cecr2 | Longitudinal | Igfbp1 |
| Upregulation | | Downregulation | | Increase | |
| Sustained | Grpel2 | Sustained | Ly6e | Longitudinal | Ifrd1 |
| Upregulation | | Downregulation | | Increase | |
| Sustained | Ido2 | Sustained | Acaca | Longitudinal | Tpm1 |
| Upregulation | | Downregulation | | Increase | |
| Sustained | Zscan26 | Sustained | Txndc11 | Longitudinal | Gdf15 |
| Upregulation | | Downregulation | | Increase | |
| Sustained | Hdac11 | Sustained | Bach2 | Longitudinal | Krt8 |
| Upregulation | | Downregulation | | Increase | |
| Sustained | Tbl1xr1 | Sustained | Il13ra1 | Longitudinal | Ldlr |
| Upregulation | | Downregulation | | Increase | |
| Sustained | Trp53inp1 | Sustained | Aspg | Longitudinal | Rnf213 |
| Upregulation | | Downregulation | | Increase | |
| Sustained | Ccdc107 | Sustained | Pla1a | Longitudinal | Srgap2 |
| Upregulation | | Downregulation | | Increase | |
| Sustained | Dram2 | Sustained | Prdx4 | Longitudinal | Mvp |
| Upregulation | | Downregulation | | Increase | |
| Sustained | Txnip | Sustained | Cxadr | Longitudinal | Lsamp |
| Upregulation | | Downregulation | | Increase | |
| Sustained | Cog4 | Sustained | Itih2 | Longitudinal | Dnajc10 |
| Upregulation | | Downregulation | | Increase | |
| Sustained | Ogt | Sustained | Gcat | Longitudinal | Fhit |
| Upregulation | | Downregulation | | Increase | |
| Sustained | Pde1c | Sustained | Egfr | Longitudinal | Chrm3 |
| Upregulation | | Downregulation | | Increase | |
| Sustained | Gpat4 | Sustained | Enpp3 | Longitudinal | B4galnt1 |
| Upregulation | | Downregulation | | Increase | |
| Sustained | Vps41 | Sustained | Ern1 | Longitudinal | Crip2 |
| Upregulation | | Downregulation | | Increase | |
| Sustained | Tmem87b | Sustained | Hs3st3b1 | Longitudinal | Sptan1 |
| Upregulation | | Downregulation | | Increase | |
| Sustained | Abcc3 | Sustained | Gltpd2 | Longitudinal | Nsdhl |
| Upregulation | | Downregulation | | Increase | |
| Sustained | Tsc22d1 | Sustained | Esr1 | Longitudinal | Pttg1 |
| Upregulation | | Downregulation | | Increase | |
| Sustained | Smarca4 | Sustained | Nos1ap | Longitudinal | Zap70 |
| Upregulation | | Downregulation | | Increase | |
| Sustained | Mdm2 | Sustained | Pemt | Longitudinal | Atp8b1 |
| Upregulation | | Downregulation | | Increase | |
| Sustained | Gna12 | Sustained | Btd | Longitudinal | Gabbr2 |
| Upregulation | | Downregulation | | Increase | |
| Sustained | Glrx | Sustained | Apol9a | Longitudinal | Spint2 |
| Upregulation | | Downregulation | | Increase | |
| Sustained | Sod2 | Sustained | Hc | Longitudinal | Lgals3bp |
| Upregulation | | Downregulation | | Increase | |
| Sustained | Fitm2 | Sustained | Hes6 | Longitudinal | Ip6k2 |
| Upregulation | | Downregulation | | Increase | |
| Sustained | Gstt2 | Sustained | Sdf2l1 | Longitudinal | Ogfrl1 |
| Upregulation | | Downregulation | | Increase | |
| Sustained | Idh2 | Sustained | Stt3a | Longitudinal | Dynll1 |
| Upregulation | | Downregulation | | Increase | |
| Sustained | Stx7 | Sustained | Atp2a2 | Longitudinal | H2-Ab1 |
| Upregulation | | Downregulation | | Increase | |
| Sustained | Rnd3 | Sustained | Spcs2 | Longitudinal | Tshz2 |
| Upregulation | | Downregulation | | Increase | |
| Sustained | Cd1d1 | Sustained | Serpina1e | Longitudinal | Rdh11 |
| Upregulation | | Downregulation | | Increase | |
| Sustained | Mmp19 | Sustained | Masp1 | Longitudinal | Cldn2 |
| Upregulation | | Downregulation | | Increase | |
| Sustained | Mpzl2 | Sustained | Aadac | Longitudinal | Clec4f |
| Upregulation | | Downregulation | | Increase | |
| Sustained | Ift52 | Sustained | Lifr | Longitudinal | Arl15 |
| Upregulation | | Downregulation | | Increase | |
| Sustained | 1600014C10Rik | Sustained | F7 | Longitudinal | Kyat1 |
| Upregulation | | Downregulation | | Increase | |
| Sustained | Zfand5 | Sustained | 0610040J01Rik | Longitudinal | Ep400 |
| Upregulation | | Downregulation | | Increase | |
| Sustained | Sgk2 | Sustained | Mogs | Longitudinal | Mrap |
| Upregulation | | Downregulation | | Increase | |
| Sustained | Myl6 | Sustained | Rpl35 | Longitudinal | Krtcap2 |
| Upregulation | | Downregulation | | Increase | |
| Sustained | Vwa5a | Sustained | Tuba4a | Longitudinal | Fyb2 |
| Upregulation | | Downregulation | | Increase | |
| Sustained | Abcb4 | Sustained | Gpr146 | Longitudinal | Zfp36 |
| Upregulation | | Downregulation | | Increase | |
| Sustained | Blvra | Sustained | Apol9b | Longitudinal | Apbb2 |
| Upregulation | | Downregulation | | Increase | |
| Sustained | Chkb | Sustained | Mgat2 | Longitudinal | Rras |
| Upregulation | | Downregulation | | Increase | |
| Sustained | Ddx52 | Sustained | Abhd17b | Longitudinal | Alcam |
| Upregulation | | Downregulation | | Increase | |
| Sustained | Decr1 | Sustained | Fuom | Longitudinal | Suco |
| Upregulation | | Downregulation | | Increase | |
| Sustained | Pts | Sustained | Ssr4 | Longitudinal | Hspa4l |
| Upregulation | | Downregulation | | Increase | |
| Sustained | Zfp655 | Sustained | Sec23b | Longitudinal | Pdlim1 |
| Upregulation | | Downregulation | | Increase | |
| Sustained | Ppdpf | Sustained | Sec63 | Longitudinal | Steap4 |
| Upregulation | | Downregulation | | Increase | |
| Sustained | Aifm2 | Sustained | Uso1 | Longitudinal | Vwa5a |
| Upregulation | | Downregulation | | Increase | |
| Sustained | Hpgd | Sustained | Psmb10 | Longitudinal | Manf |
| Upregulation | | Downregulation | | Increase | |
| Sustained | Them4 | Sustained | Fkbp11 | Longitudinal | Tmem97 |
| Upregulation | | Downregulation | | Increase | |
| Sustained | Arpc5 | Sustained | Hspa5 | Longitudinal | Capn2 |
| Upregulation | | Downregulation | | Increase | |
| Sustained | Celf2 | Sustained | Xbp1 | Longitudinal | Ppp1r9a |
| Upregulation | | Downregulation | | Increase | |
| Sustained | Stard5 | Sustained | Stbd1 | Longitudinal | Slc16a12 |
| Upregulation | | Downregulation | | Increase | |
| Sustained | Gpd2 | Sustained | Slc6a13 | Longitudinal | Aldh1b1 |
| Upregulation | | Downregulation | | Increase | |
| Sustained | Coa6 | Sustained | Ddc | Longitudinal | Skap2 |
| Upregulation | | Downregulation | | Increase | |
| Sustained | Mtor | Sustained | C2 | Longitudinal | Tmem30b |
| Upregulation | | Downregulation | | Increase | |
| Sustained | Pgm3 | Sustained | Cyp4v3 | Longitudinal | Tmem87b |
| Upregulation | | Downregulation | | Increase | |
| Sustained | Ipmk | Sustained | Cyp4f14 | Longitudinal | Vps13a |
| Upregulation | | Downregulation | | Increase | |
| Sustained | Ptgr2 | Sustained | Osbp | Longitudinal | Colec12 |
| Upregulation | | Downregulation | | Increase | |
| Sustained | Bod1l | Sustained | Ces2a | Longitudinal | Fgfr2 |
| Upregulation | | Downregulation | | Increase | |
| Sustained | Actb | Sustained | Irf7 | Longitudinal | Akr1e1 |
| Upregulation | | Downregulation | | Increase | |
| Sustained | Gcc2 | Sustained | Inhbc | Longitudinal | Elovl1 |
| Upregulation | | Downregulation | | Increase | |
| Sustained | Cmip | Sustained | Avpi1 | Longitudinal | mt-Nd6 |
| Upregulation | | Downregulation | | Increase | |
| Sustained | Rcbtb1 | Sustained | Cadps2 | Longitudinal | Arl6ip5 |
| Upregulation | | Downregulation | | Increase | |
| Sustained | C77080 | Sustained | Hspa13 | Longitudinal | Dhcr7 |
| Upregulation | | Downregulation | | Increase | |
| Sustained | Fmr1 | Sustained | Ostc | Longitudinal | Gstp1 |
| Upregulation | | Downregulation | | Increase | |
| Sustained | Plekha8 | Sustained | C1ra | Longitudinal | Litaf |
| Upregulation | | Downregulation | | Increase | |
| Sustained | Perp | Sustained | Cyb5r3 | Longitudinal | Abhd12 |
| Upregulation | | Downregulation | | Increase | |
| Sustained | Ndufa7 | Sustained | Pter | Longitudinal | Rnf19a |
| Upregulation | | Downregulation | | Increase | |
| Sustained | Hexa | Sustained | Os9 | Longitudinal | Fdft1 |
| Upregulation | | Downregulation | | Increase | |
| Sustained | Cpt1a | Sustained | Tars | Longitudinal | Fam3c |
| Upregulation | | Downregulation | | Increase | |
| Sustained | Mycbp | Sustained | Hyou1 | Longitudinal | Jund |
| Upregulation | | Downregulation | | Increase | |
| Sustained | Apip | Sustained | Phlpp1 | Longitudinal | Rangap1 |
| Upregulation | | Downregulation | | Increase | |
| Sustained | Gtf2h5 | Sustained | Surf4 | Longitudinal | Ugt3a2 |
| Upregulation | | Downregulation | | Increase | |
| Sustained | Dhrs7 | Sustained | Paics | Longitudinal | Dlgap1 |
| Upregulation | | Downregulation | | Increase | |
| Sustained | Mrpl27 | Sustained | Pcgf5 | Longitudinal | Pm20d1 |
| Upregulation | | Downregulation | | Increase | |
| Sustained | Ndufv2 | Sustained | Creld2 | Longitudinal | Habp4 |
| Upregulation | | Downregulation | | Increase | |
| Sustained | Arpc3 | Sustained | B4galt5 | Longitudinal | Akap8l |
| Upregulation | | Downregulation | | Increase | |
| Sustained | Fbxo31 | Sustained | Entpd8 | Longitudinal | Slc35g1 |
| Upregulation | | Downregulation | | Increase | |
| Sustained | Vgll4 | Sustained | Zfp609 | Longitudinal | Pard3b |
| Upregulation | | Downregulation | | Increase | |
| Sustained | Rhob | Sustained | Cmtm6 | Longitudinal | Hspb8 |
| Upregulation | | Downregulation | | Increase | |
| Sustained | Clmn | Sustained | Raph1 | Longitudinal | Tcf4 |
| Upregulation | | Downregulation | | Increase | |
| Sustained | Trak1 | Sustained | Ninj1 | Longitudinal | Rasal2 |
| Upregulation | | Downregulation | | Increase | |
| Sustained | Lrmda | Sustained | Qdpr | Longitudinal | Svil |
| Upregulation | | Downregulation | | Increase | |
| Sustained | Micu2 | Sustained | Atp9b | Longitudinal | Ppt1 |
| Upregulation | | Downregulation | | Increase | |
| Sustained | Chdh | Sustained | Arhgap32 | Longitudinal | Chka |
| Upregulation | | Downregulation | | Increase | |
| Sustained | Zbtb38 | Sustained | Erp44 | Longitudinal | Slc38a2 |
| Upregulation | | Downregulation | | Increase | |
| Sustained | Vma21 | Sustained | Ganab | Longitudinal | Erc2 |
| Upregulation | | Downregulation | | Increase | |
| Sustained | Dennd4a | Sustained | Zfp622 | Longitudinal | Sfswap |
| Upregulation | | Downregulation | | Increase | |
| Sustained | Sirt7 | Sustained | Mbl2 | Longitudinal | Tm7sf2 |
| Upregulation | | Downregulation | | Increase | |
| Sustained | Mob1b | Sustained | Slc16a1 | Longitudinal | Bmi1 |
| Upregulation | | Downregulation | | Increase | |
| Sustained | 2810004N23Rik | Sustained | Rabac1 | Longitudinal | Chd7 |
| Upregulation | | Downregulation | | Increase | |
| Sustained | Sptssa | Sustained | Dpy19l1 | Longitudinal | Irf2bp2 |
| Upregulation | | Downregulation | | Increase | |
| Sustained | Rbm17 | Sustained | F11r | Longitudinal | Slc6a6 |
| Upregulation | | Downregulation | | Increase | |
| Sustained | Ube2w | Sustained | Hdhd3 | Longitudinal | Npc1 |
| Upregulation | | Downregulation | | Increase | |
| Sustained | Snrk | Sustained | Sidt2 | Longitudinal | Mat2a |
| Upregulation | | Downregulation | | Increase | |
| Sustained | Cisd1 | Sustained | Cldn3 | Longitudinal | Kalrn |
| Upregulation | | Downregulation | | Increase | |
| Sustained | Inf2 | Sustained | Stard13 | Longitudinal | Tm4sf4 |
| Upregulation | | Downregulation | | Increase | |
| Sustained | Ttc7 | Sustained | Foxa3 | Longitudinal | Arhgef19 |
| Upregulation | | Downregulation | | Increase | |
| Sustained | Cyld | Sustained | Smpd1 | Longitudinal | Lmna |
| Upregulation | | Downregulation | | Increase | |
| Sustained | Pim3 | Sustained | Mid1ip1 | Longitudinal | Cnpy2 |
| Upregulation | | Downregulation | | Increase | |
| Sustained | Triap1 | Sustained | Atxn1 | Longitudinal | Tgfbr1 |
| Upregulation | | Downregulation | | Increase | |
| Sustained | Pdcd6 | Sustained | Yipf3 | Longitudinal | Nr6a1 |
| Upregulation | | Downregulation | | Increase | |
| Sustained | Nat9 | Sustained | Samm50 | Longitudinal | Asap1 |
| Upregulation | | Downregulation | | Increase | |
| Sustained | Trnau1ap | Sustained | Nudt19 | Longitudinal | Ctsa |
| Upregulation | | Downregulation | | Increase | |
| Sustained | Lactb | Sustained | Endog | Longitudinal | Anxa5 |
| Upregulation | | Downregulation | | Increase | |
| Sustained | Snapc5 | Sustained | Dvl1 | Longitudinal | Tlcd1 |
| Upregulation | | Downregulation | | Increase | |
| Sustained | Snx10 | Sustained | Pla2g12b | Longitudinal | Erbb4 |
| Upregulation | | Downregulation | | Increase | |
| Sustained | Arhgap12 | Sustained | Ppm1g | Longitudinal | Fdps |
| Upregulation | | Downregulation | | Increase | |
| Sustained | Rcbtb2 | Sustained | Ktn1 | Longitudinal | Fndc4 |
| Upregulation | | Downregulation | | Increase | |
| Sustained | Higd1a | Sustained | Larp1 | Longitudinal | Sorbs2 |
| Upregulation | | Downregulation | | Increase | |
| Sustained | Slc48a1 | Sustained | Acp1 | Longitudinal | Mcl1 |
| Upregulation | | Downregulation | | Increase | |
| Sustained | Taok1 | Sustained | Txndc12 | Longitudinal | Cacul1 |
| Upregulation | | Downregulation | | Increase | |
| Sustained | Dcun1d1 | Sustained | Plekhj1 | Longitudinal | Ifnar2 |
| Upregulation | | Downregulation | | Increase | |
| Sustained | Mapk1ip1l | Sustained | Cox7a2 | Longitudinal | Hypk |
| Upregulation | | Downregulation | | Increase | |
| Sustained | Xpa | Sustained | Clic4 | Longitudinal | Mast4 |
| Upregulation | | Downregulation | | Increase | |
| Sustained | Gnpat | Sustained | Emc10 | Longitudinal | Hsd17b13 |
| Upregulation | | Downregulation | | Increase | |
| Sustained | Tmem242 | Sustained | App | Longitudinal | Zmiz1 |
| Upregulation | | Downregulation | | Increase | |
| Sustained | Dpp8 | Sustained | Id2 | Longitudinal | Mbnl2 |
| Upregulation | | Downregulation | | Increase | |
| Sustained | Pfdn1 | Sustained | Tmem53 | Longitudinal | Rfx7 |
| Upregulation | | Downregulation | | Increase | |
| Sustained | Mrpl1 | Sustained | H13 | Longitudinal | Pi4k2a |
| Upregulation | | Downregulation | | Increase | |
| Sustained | Ndufaf6 | Sustained | Preb | Longitudinal | Pan3 |
| Upregulation | | Downregulation | | Increase | |
| Sustained | Commd6 | Sustained | Cars | Longitudinal | Hjurp |
| Upregulation | | Downregulation | | Increase | |
| Sustained | Pcf11 | Sustained | Rps21 | Longitudinal | Phf21a |
| Upregulation | | Downregulation | | Increase | |
| Sustained | R3hdm4 | Sustained | Chmp7 | Longitudinal | Ppp1r15a |
| Upregulation | | Downregulation | | Increase | |
| Sustained | Cntrl | Sustained | Tmed9 | Longitudinal | Mrtfb |
| Upregulation | | Downregulation | | Increase | |
| Sustained | Hipk3 | Sustained | Kif13b | Longitudinal | Parp12 |
| Upregulation | | Downregulation | | Increase | |
| Sustained | Chmp1a | Sustained | Ubr2 | Longitudinal | Fkbp1a |
| Upregulation | | Downregulation | | Increase | |
| Sustained | Smpd2 | Sustained | Tmem248 | Longitudinal | Hsph1 |
| Upregulation | | Downregulation | | Increase | |
| Sustained | Bola3 | Sustained | Plec | Longitudinal | Id3 |
| Upregulation | | Downregulation | | Increase | |
| Sustained | Fem1a | Sustained | Rpl38 | Longitudinal | Gpr89 |
| Upregulation | | Downregulation | | Increase | |
| Sustained | Josd1 | Sustained | Fam47e | Longitudinal | Neu1 |
| Upregulation | | Downregulation | | Increase | |
| Sustained | Ankib1 | Sustained | Amdhd1 | Longitudinal | Nrg4 |
| Upregulation | | Downregulation | | Increase | |
| Sustained | Phpt1 | Sustained | Zcchc7 | Longitudinal | Tsc22d2 |
| Upregulation | | Downregulation | | Increase | |
| Sustained | Stx4a | Sustained | Agtr1a | Longitudinal | Map4 |
| Upregulation | | Downregulation | | Increase | |
| Sustained | Ndufa5 | Sustained | Mycbp2 | Longitudinal | Nrbp2 |
| Upregulation | | Downregulation | | Increase | |
| Sustained | Rufy 1 | Sustained | Amfr | Longitudinal | Cd74 |
| Upregulation | | Downregulation | | Increase | |
| Sustained | Qrich1 | Sustained | Mal2 | Longitudinal | Rbpms |
| Upregulation | | Downregulation | | Increase | |
| Sustained | Rest | Sustained | Slc30a5 | Longitudinal | Atp6ap2 |
| Upregulation | | Downregulation | | Increase | |
| Sustained | Fbxo11 | Sustained | Yars | Longitudinal | Nlrc5 |
| Upregulation | | Downregulation | | Increase | |
| Sustained | Trappc5 | Sustained | Ubfd1 | Longitudinal | Hsd17b7 |
| Upregulation | | Downregulation | | Increase | |
| Sustained | Polr2f | Sustained | Dnajb9 | Longitudinal | Nav2 |
| Upregulation | | Downregulation | | Increase | |
| Sustained | Ssh2 | Sustained | Tbc1d5 | Longitudinal | Nme1 |
| Upregulation | | Downregulation | | Increase | |
| Sustained | Sdccag8 | Sustained | Slc35e1 | Longitudinal | Actb |
| Upregulation | | Downregulation | | Increase | |
| Sustained | Klhl9 | Sustained | Rbfox2 | Longitudinal | Neb |
| Upregulation | | Downregulation | | Increase | |
| Sustained | Bod1 | Sustained | Zdhhc9 | Longitudinal | Tuba1c |
| Upregulation | | Downregulation | | Increase | |
| Sustained | Atp6v1g1 | Sustained | Tmem37 | Longitudinal | Hmgcs1 |
| Upregulation | | Downregulation | | Increase | |
| | | Sustained | Adam9 | Longitudinal | Arpc4 |
| | | Downregulation | | Increase | |
| | | Sustained | Dmxl1 | Longitudinal | Eif4a1 |
| | | Downregulation | | Increase | |
| | | Sustained | Znrf2 | Longitudinal | Abhd13 |
| | | Downregulation | | Increase | |
| | | Sustained | Derl1 | Longitudinal | Trmt1 |
| | | Downregulation | | Increase | |
| | | Sustained | Scaf8 | Longitudinal | Cstb |
| | | Downregulation | | Increase | |
| | | Sustained | Nsun2 | Longitudinal | Gas6 |
| | | Downregulation | | Increase | |
| | | Sustained | Hint1 | Longitudinal | Ogt |
| | | Downregulation | | Increase | |
| | | Sustained | Dlc1 | Longitudinal | Osgin1 |
| | | Downregulation | | Increase | |
| | | Sustained | Slc35d2 | Longitudinal | Pgd |
| | | Downregulation | | Increase | |
| | | Sustained | Ifih1 | Longitudinal | Cnot6 |
| | | Downregulation | | Increase | |
| | | Sustained | Oxa1l | Longitudinal | Mmp19 |
| | | Downregulation | | Increase | |
| | | Sustained | 2810459M11Rik | Longitudinal | Hivep2 |
| | | Downregulation | | Increase | |
| | | Sustained | Atp11b | Longitudinal | Idh3a |
| | | Downregulation | | Increase | |
| | | Sustained | Pnpla7 | Longitudinal | Bola2 |
| | | Downregulation | | Increase | |
| | | Sustained | Kidins220 | Longitudinal | Mdm2 |
| | | Downregulation | | Increase | |
| | | Sustained | Pcmt1 | Longitudinal | Ctsc |
| | | Downregulation | | Increase | |
| | | Sustained | Stip1 | Longitudinal | Ctsd |
| | | Downregulation | | Increase | |
| | | Sustained | Numb | Longitudinal | Vmp1 |
| | | Downregulation | | Increase | |
| | | Sustained | Alg11 | Longitudinal | Peli1 |
| | | Downregulation | | Increase | |
| | | Sustained | Slc25a44 | Longitudinal | Cspp1 |
| | | Downregulation | | Increase | |
| | | Sustained | Atp1a1 | Longitudinal | Adam10 |
| | | Downregulation | | Increase | |
| | | Sustained | Mul1 | Longitudinal | Wbp1 |
| | | Downregulation | | Increase | |
| | | Sustained | Rer1 | Longitudinal | Mtmr2 |
| | | Downregulation | | Increase | |
| | | Sustained | Txndc15 | Longitudinal | Rsrp1 |
| | | Downregulation | | Increase | |
| | | Sustained | Dnajc22 | Longitudinal | Cdc16 |
| | | Downregulation | | Increase | |
| | | Sustained | Polr1d | Longitudinal | Gstm7 |
| | | Downregulation | | Increase | |
| | | Sustained | Tcf12 | Longitudinal | Arf4 |
| | | Downregulation | | Increase | |
| | | Sustained | Polr1c | Longitudinal | Lancl1 |
| | | Downregulation | | Increase | |
| | | Sustained | Brwd1 | Longitudinal | Actr1a |
| | | Downregulation | | Increase | |
| | | Sustained | Wdr33 | Longitudinal | Arpc1b |
| | | Downregulation | | Increase | |
| | | Sustained | Aven | Longitudinal | Txnrd1 |
| | | Downregulation | | Increase | |
| | | Sustained | Tmem63b | Longitudinal | Sypl |
| | | Downregulation | | Increase | |
| | | Sustained | Tmem150a | Longitudinal | Rhob |
| | | Downregulation | | Increase | |
| | | Sustained | Gdap2 | Longitudinal | Ryk |
| | | Downregulation | | Increase | |
| | | Sustained | Il6st | Longitudinal | Gorasp1 |
| | | Downregulation | | Increase | |
| | | Sustained | Wapl | Longitudinal | Chd4 |
| | | Downregulation | | Increase | |
| | | Sustained | Iah1 | Longitudinal | Ncoa6 |
| | | Downregulation | | Increase | |
| | | Sustained | Npc2 | Longitudinal | Gm2a |
| | | Downregulation | | Increase | |
| | | Sustained | Ergic2 | Longitudinal | Trappc4 |
| | | Downregulation | | Increase | |
| | | Sustained | As3mt | Longitudinal | Ppa1 |
| | | Downregulation | | Increase | |
| | | Sustained | Ddx58 | Longitudinal | Rbm6 |
| | | Downregulation | | Increase | |
| | | Sustained | Cmss1 | Longitudinal | Foxp1 |
| | | Downregulation | | Increase | |
| | | Sustained | Tnks | Longitudinal | Polr2j |
| | | Downregulation | | Increase | |
| | | Sustained | Herpud2 | Longitudinal | Cep85l |
| | | Downregulation | | Increase | |
| | | Sustained | Rpl39 | Longitudinal | Ier2 |
| | | Downregulation | | Increase | |
| | | Sustained | Wdr7 | Longitudinal | Tmx1 |
| | | Downregulation | | Increase | |
| | | Sustained | Kmt2a | Longitudinal | Nkain2 |
| | | Downregulation | | Increase | |
| | | Sustained | Acp5 | Longitudinal | Atp6v0b |
| | | Downregulation | | Increase | |
| | | Sustained | Dip2c | Longitudinal | Tasor |
| | | Downregulation | | Increase | |
| | | Sustained | Cdk12 | Longitudinal | Trak1 |
| | | Downregulation | | Increase | |
| | | Sustained | Dpp3 | Longitudinal | Tbrg1 |
| | | Downregulation | | Increase | |
| | | Sustained | Vps13b | Longitudinal | Commd2 |
| | | Downregulation | | Increase | |
| | | Sustained | Bri3bp | Longitudinal | Mrrf |
| | | Downregulation | | Increase | |
| | | Sustained | Nfat5 | Longitudinal | Tspo |
| | | Downregulation | | Increase | |
| | | Sustained | Slc25a17 | Longitudinal | Tmem243 |
| | | Downregulation | | Increase | |
| | | Sustained | Tnrc6c | Longitudinal | Lbp |
| | | Downregulation | | Increase | |
| | | | | Longitudinal | Tmbim4 |
| | | | | Increase | |
| | | | | Longitudinal | Nop58 |
| | | | | Increase | |
| | | | | Longitudinal | Zfc3h1 |
| | | | | Increase | |
| | | | | Longitudinal | Gnl2 |
| | | | | Increase | |
| | | | | Longitudinal | Sema4a |
| | | | | Increase | |
| | | | | Longitudinal | Tspan4 |
| | | | | Increase | |
| | | | | Longitudinal | Il1r1 |
| | | | | Increase | |
| | | | | Longitudinal | Cd151 |
| | | | | Increase | |
| | | | | Longitudinal | Mdm4 |
| | | | | Increase | |
| | | | | Longitudinal | Sc5d |
| | | | | Increase | |
| | | | | Longitudinal | Chsy3 |
| | | | | Increase | |
| | | | | Longitudinal | Rnf149 |
| | | | | Increase | |
| | | | | Longitudinal | Ebna1bp2 |
| | | | | Increase | |
| | | | | Longitudinal | Twsg1 |
| | | | | Increase | |
| | | | | Longitudinal | Wipi1 |
| | | | | Increase | |
| | | | | Longitudinal | Daam1 |
| | | | | Increase | |
| | | | | Longitudinal | Fus |
| | | | | Increase | |
| | | | | Longitudinal | Pafah1b2 |
| | | | | Increase | |
| | | | | Longitudinal | Atp6v0e |
| | | | | Increase | |
| | | | | Longitudinal | Aopep |
| | | | | Increase | |
| | | | | Longitudinal | Hnrnpab |
| | | | | Increase | |
| | | | | Longitudinal | Dnajb11 |
| | | | | Increase | |
| | | | | Longitudinal | Grb14 |
| | | | | Increase | |
| | | | | Longitudinal | Cdk5rap3 |
| | | | | Increase | |
| | | | | Longitudinal | Gpatch8 |
| | | | | Increase | |
| | | | | Longitudinal | Stx18 |
| | | | | Increase | |
| | | | | Longitudinal | Anxa4 |
| | | | | Increase | |
| | | | | Longitudinal | Gadd45g |
| | | | | Increase | |
| | | | | Longitudinal | Ywhaz |
| | | | | Increase | |
| | | | | Longitudinal | Sra1 |
| | | | | Increase | |
| | | | | Longitudinal | Asph |
| | | | | Increase | |
| | | | | Longitudinal | Arl8b |
| | | | | Increase | |
| | | | | Longitudinal | Hyal2 |
| | | | | Increase | |
| | | | | Longitudinal | Fam98a |
| | | | | Increase | |
| | | | | Longitudinal | Calm2 |
| | | | | Increase | |
| | | | | Longitudinal | Srsf2 |
| | | | | Increase | |
| | | | | Longitudinal | Hopx |
| | | | | Increase | |
| | | | | Longitudinal | Pgm3 |
| | | | | Increase | |
| | | | | Longitudinal | Fbxo22 |
| | | | | Increase | |
| | | | | Longitudinal | Zmym2 |
| | | | | Increase | |
| | | | | Longitudinal | Ccz1 |
| | | | | Increase | |
| | | | | Longitudinal | Tmed7 |
| | | | | Increase | |
| | | | | Longitudinal | Pet100 |
| | | | | Increase | |
| | | | | Longitudinal | Gins4 |
| | | | | Increase | |
| | | | | Longitudinal | Iars |
| | | | | Increase | |
| | | | | Longitudinal | Etnk1 |
| | | | | Increase | |
| | | | | Longitudinal | Abhd16a |
| | | | | Increase | |
| | | | | Longitudinal | Il11ra1 |
| | | | | Increase | |
| | | | | Longitudinal | Rdh14 |
| | | | | Increase | |
| | | | | Longitudinal | Lrmda |
| | | | | Increase | |
| | | | | Longitudinal | Bub3 |
| | | | | Increase | |
| | | | | Longitudinal | Abca3 |
| | | | | Increase | |
| | | | | Longitudinal | Spin1 |
| | | | | Increase | |
| | | | | Longitudinal | Tmem50a |
| | | | | Increase | |
| | | | | Longitudinal | Rnft1 |
| | | | | Increase | |
| | | | | Longitudinal | Mybbp1a |
| | | | | Increase | |
| | | | | Longitudinal | Rbck1 |
| | | | | Increase | |
| | | | | Longitudinal | Pdzd11 |
| | | | | Increase | |
| | | | | Longitudinal | Mpzl2 |
| | | | | Increase | |
| | | | | Longitudinal | Vps13c |
| | | | | Increase | |
| | | | | Longitudinal | Insig1 |
| | | | | Increase | |
| | | | | Longitudinal | Srsf3 |
| | | | | Increase | |
| | | | | Longitudinal | Tmem33 |
| | | | | Increase | |
| | | | | Longitudinal | Baz2b |
| | | | | Increase | |
| | | | | Longitudinal | Ddx17 |
| | | | | Increase | |
| | | | | Longitudinal | Arpc5 |
| | | | | Increase | |
| | | | | Longitudinal | Vamp8 |
| | | | | Increase | |
| | | | | Longitudinal | Mif4gd |
| | | | | Increase | |
| | | | | Longitudinal | Tmem147 |
| | | | | Increase | |
| | | | | Longitudinal | Rtp4 |
| | | | | Increase | |
| | | | | Longitudinal | Egln2 |
| | | | | Increase | |
| | | | | Longitudinal | Itgb5 |
| | | | | Increase | |
| | | | | Longitudinal | Ankle2 |
| | | | | Increase | |
| | | | | Longitudinal | Mark3 |
| | | | | Increase | |
| | | | | Longitudinal | Dnajc2 |
| | | | | Increase | |
| | | | | Longitudinal | Lclat1 |
| | | | | Increase | |
| | | | | Longitudinal | Tmcc1 |
| | | | | Increase | |
| | | | | Longitudinal | Rnf115 |
| | | | | Increase | |
| | | | | Longitudinal | Khdc4 |
| | | | | Increase | |
| | | | | Longitudinal | Bcl2l1 |
| | | | | Increase | |
| | | | | Longitudinal | Uba3 |
| | | | | Increase | |
| | | | | Longitudinal | Ppp3cb |
| | | | | Increase | |
| | | | | Longitudinal | Sptssa |
| | | | | Increase | |
| | | | | Longitudinal | Yipf1 |
| | | | | Increase | |
| | | | | Longitudinal | Trp53inp1 |
| | | | | Increase | |
| | | | | Longitudinal | Pnrc1 |
| | | | | Increase | |
| | | | | Longitudinal | Arfip1 |
| | | | | Increase | |
| | | | | Longitudinal | Dnm1l |
| | | | | Increase | |
| | | | | Longitudinal | Atf4 |
| | | | | Increase | |
| | | | | Longitudinal | Stat6 |
| | | | | Increase | |
| | | | | Longitudinal | Slc35e2 |
| | | | | Increase | |
| | | | | Longitudinal | Slc48a1 |
| | | | | Increase | |
| | | | | Longitudinal | Tcf7l2 |
| | | | | Increase | |
| | | | | Longitudinal | Txndc5 |
| | | | | Increase | |
| | | | | Longitudinal | Ddx52 |
| | | | | Increase | |
| | | | | Longitudinal | Cdkal1 |
| | | | | Increase | |
| | | | | Longitudinal | Caprin1 |
| | | | | Increase | |
| | | | | Longitudinal | Cct6a |
| | | | | Increase | |
| | | | | Longitudinal | Midn |
| | | | | Increase | |
| | | | | Longitudinal | Txnip |
| | | | | Increase | |
| | | | | Longitudinal | Pik3c2a |
| | | | | Increase | |
| | | | | Longitudinal | Ptrh2 |
| | | | | Increase | |
| | | | | Longitudinal | Srsf7 |
| | | | | Increase | |
| | | | | Longitudinal | Gna12 |
| | | | | Increase | |
| | | | | Longitudinal | Atp6v1e1 |
| | | | | Increase | |
| | | | | Longitudinal | Fitm2 |
| | | | | Increase | |
| | | | | Longitudinal | Pnrc2 |
| | | | | Increase | |
| | | | | Longitudinal | Rbx1 |
| | | | | Increase | |
| | | | | Longitudinal | Ppp2r3a |
| | | | | Increase | |
| | | | | Longitudinal | Jkamp |
| | | | | Increase | |
| | | | | Longitudinal | Eif2b4 |
| | | | | Increase | |
| | | | | Longitudinal | Ankib1 |
| | | | | Increase | |
| | | | | Longitudinal | Sacm1l |
| | | | | Increase | |
| | | | | Longitudinal | Babam2 |
| | | | | Increase | |
| | | | | Longitudinal | Abcf2 |
| | | | | Increase | |
| | | | | Longitudinal | Kras |
| | | | | Increase | |
| | | | | Longitudinal | Hnrnpdl |
| | | | | Increase | |
| | | | | Longitudinal | Pacsin2 |
| | | | | Increase | |
| | | | | Longitudinal | Tm7sf3 |
| | | | | Increase | |
| | | | | Longitudinal | Dstn |
| | | | | Increase | |
| | | | | Longitudinal | Gns |
| | | | | Increase | |
| | | | | Longitudinal | Afdn |
| | | | | Increase | |
| | | | | Longitudinal | Sec16a |
| | | | | Increase | |
| | | | | Longitudinal | Rassf8 |
| | | | | Increase | |
| | | | | Longitudinal | Ror1 |
| | | | | Increase | |
| | | | | Longitudinal | Rere |
| | | | | Increase | |
| | | | | Longitudinal | Lss |
| | | | | Increase | |
| | | | | Longitudinal | Cib1 |
| | | | | Increase | |
| | | | | Longitudinal | Pa2g4 |
| | | | | Increase | |
| | | | | Longitudinal | Vti1a |
| | | | | Increase | |
| | | | | Longitudinal | Rars |
| | | | | Increase | |
| | | | | Longitudinal | Heatr3 |
| | | | | Increase | |
| | | | | Longitudinal | Ppp2r2a |
| | | | | Increase | |
| | | | | Longitudinal | Cgrrf1 |
| | | | | Increase | |
| | | | | Longitudinal | Casp3 |
| | | | | Increase | |
| | | | | Longitudinal | Zfp638 |
| | | | | Increase | |
| | | | | Longitudinal | Atl3 |
| | | | | Increase | |
| | | | | Longitudinal | Plrg1 |
| | | | | Increase | |
| | | | | Longitudinal | Tm2d3 |
| | | | | Increase | |
| | | | | Longitudinal | Scamp1 |
| | | | | Increase | |
| | | | | Longitudinal | Utp6 |
| | | | | Increase | |
| | | | | Longitudinal | Nsmce2 |
| | | | | Increase | |
| | | | | Longitudinal | Taf12 |
| | | | | Increase | |
| | | | | Longitudinal | Fam193a |
| | | | | Increase | |
| | | | | Longitudinal | Dsc2 |
| | | | | Increase | |
| | | | | Longitudinal | Flot1 |
| | | | | Increase | |
| | | | | Longitudinal | Lasp1 |
| | | | | Increase | |
| | | | | Longitudinal | Srpk1 |
| | | | | Increase | |
| | | | | Longitudinal | Edem3 |
| | | | | Increase | |
| | | | | Longitudinal | Ormdl2 |
| | | | | Increase | |
| | | | | Longitudinal | Cs |
| | | | | Increase | |
| | | | | Longitudinal | Cct2 |
| | | | | Increase | |
| | | | | Longitudinal | Pon2 |
| | | | | Increase | |
| | | | | Longitudinal | BC031181 |
| | | | | Increase | |
| | | | | Longitudinal | Suz12 |
| | | | | Increase | |
| | | | | Longitudinal | Vma21 |
| | | | | Increase | |
| | | | | Longitudinal | Ube2g2 |
| | | | | Increase | |
| | | | | Longitudinal | Trnau1ap |
| | | | | Increase | |
| | | | | Longitudinal | Lsm4 |
| | | | | Increase | |
| | | | | Longitudinal | Usp3 |
| | | | | Increase | |
| | | | | Longitudinal | Impa1 |
| | | | | Increase | |
| | | | | Longitudinal | Uhrf2 |
| | | | | Increase | |
| | | | | Longitudinal | 1700066M21Rik |
| | | | | Increase | |
| | | | | Longitudinal | Aga |
| | | | | Increase | |
| | | | | Longitudinal | Ftsj3 |
| | | | | Increase | |
| | | | | Longitudinal | Nars |
| | | | | Increase | |
| | | | | Longitudinal | Pxk |
| | | | | Increase | |
| | | | | Longitudinal | Ube2w |
| | | | | Increase | |
| | | | | Longitudinal | Phpt1 |
| | | | | Increase | |
| | | | | Longitudinal | Tnfaip1 |
| | | | | Increase | |
| | | | | Longitudinal | Plaa |
| | | | | Increase | |
| | | | | Longitudinal | Nfyb |
| | | | | Increase | |
| | | | | Longitudinal | Sec22b |
| | | | | Increase | |
| | | | | Longitudinal | Nras |
| | | | | Increase | |
| | | | | Longitudinal | Atad2b |
| | | | | Increase | |
| | | | | Longitudinal | Acss2 |
| | | | | Increase | |
| | | | | Longitudinal | Psmd9 |
| | | | | Increase | |
| | | | | Longitudinal | Mrpl50 |
| | | | | Increase | |
| | | | | Longitudinal | Tmod3 |
| | | | | Increase | |
| | | | | Longitudinal | Golga3 |
| | | | | Increase | |
| | | | | Longitudinal | Snapc5 |
| | | | | Increase | |
| | | | | Longitudinal | Znrf1 |
| | | | | Increase | |
| | | | | Longitudinal | Clock |
| | | | | Increase | |
| | | | | Longitudinal | Abcb4 |
| | | | | Increase | |
| | | | | Longitudinal | Rc3h1 |
| | | | | Increase | |
| | | | | Longitudinal | Bms1 |
| | | | | Increase | |
| | | | | Longitudinal | Uqcc1 |
| | | | | Increase | |
| | | | | Longitudinal | Rnf146 |
| | | | | Increase | |
| | | | | Longitudinal | Rexo2 |
| | | | | Increase | |
| | | | | Longitudinal | Galnt1 |
| | | | | Increase | |
| | | | | Longitudinal | Sdcbp |
| | | | | Increase | |
| | | | | Longitudinal | Siae |
| | | | | Increase | |
| | | | | Longitudinal | Ptp4a1 |
| | | | | Increase | |
| | | | | Longitudinal | Lztr1 |
| | | | | Increase | |
| | | | | Longitudinal | Polr2f |
| | | | | Increase | |
| | | | | Longitudinal | 1700123O20Rik |
| | | | | Increase | |
| | | | | Longitudinal | Ist1 |
| | | | | Increase | |
| | | | | Longitudinal | Cops8 |
| | | | | Increase | |
| | | | | Longitudinal | Cdc42 |
| | | | | Increase | |
| | | | | Longitudinal | Tdrd7 |
| | | | | Increase | |
| | | | | Longitudinal | Cntrl |
| | | | | Increase | |
| | | | | Longitudinal | Gars |
| | | | | Increase | |
| | | | | Longitudinal | Trappc5 |
| | | | | Increase | |
| | | | | Longitudinal | Ddx3x |
| | | | | Increase | |
| | | | | Longitudinal | Rpf1 |
| | | | | Increase | |
| | | | | Longitudinal | Triap1 |
| | | | | Increase | |
| | | | | Longitudinal | Cript |
| | | | | Increase | |
| | | | | Longitudinal | Ttc7 |
| | | | | Increase | |
| | | | | Longitudinal | Hsp90aa1 |
| | | | | Increase | |
| | | | | Longitudinal | Mapre1 |
| | | | | Increase | |
| | | | | Longitudinal | Ndufaf4 |
| | | | | Increase | |
| | | | | Longitudinal | Pfdn1 |
| | | | | Increase | |
| | | | | Longitudinal | Ppp6r3 |
| | | | | Increase | |
| | | | | Longitudinal | Thumpd1 |
| | | | | Increase | |
| | | | | Longitudinal | Eif2b1 |
| | | | | Increase | |
| | | | | Longitudinal | Ddx27 |
| | | | | Increase | |
| | | | | Longitudinal | Otud5 |
| | | | | Increase | |
| | | | | Longitudinal | Morf4l2 |
| | | | | Increase | |
| | | | | Longitudinal | Cnot4 |
| | | | | Increase | |
| | | | | Longitudinal | Cnn3 |
| | | | | Increase | |
| | | | | Longitudinal | Carmil1 |
| | | | | Increase | |
| | | | | Longitudinal | Tiprl |
| | | | | Increase | |
| | | | | Longitudinal | Tmx2 |
| | | | | Increase | |
| | | | | Longitudinal | Hexa |
| | | | | Increase | |
| | | | | Longitudinal | Kmt2c |
| | | | | Increase | |
| | | | | Longitudinal | Dph3 |
| | | | | Increase | |
| | | | | Longitudinal | Dhx32 |
| | | | | Increase | |
| | | | | Longitudinal | Sgpp1 |
| | | | | Increase | |
| | | | | Longitudinal | Sri |
| | | | | Increase | |
| | | | | Longitudinal | Rab18 |
| | | | | Increase | |
| | | | | Longitudinal | Csnk1d |
| | | | | Increase | |
| | | | | Longitudinal | Atp6v1f |
| | | | | Increase | |
| | | | | Longitudinal | Denr |
| | | | | Increase | |
| | | | | Longitudinal | Rps4x |
| | | | | Increase | |
| | | | | Longitudinal | Pkn2 |
| | | | | Increase | |
| | | | | Longitudinal | Tatdn2 |
| | | | | Increase | |
| | | | | Longitudinal | Cct5 |
| | | | | Increase | |
| | | | | Longitudinal | Dido1 |
| | | | | Increase | |
| | | | | Longitudinal | Ppp2ca |
| | | | | Increase | |
| | | | | Longitudinal | Pitpnb |
| | | | | Increase | |
| | | | | Longitudinal | Mrps17 |
| | | | | Increase | |
| | | | | Longitudinal | Srsf1 |
| | | | | Increase | |
| | | | | Longitudinal | Rac1 |
| | | | | Increase | |
| | | | | Longitudinal | Vkorc1 |
| | | | | Increase | |
| | | | | Longitudinal | Lmo4 |
| | | | | Increase | |
| | | | | Longitudinal | Lsg1 |
| | | | | Increase | |
| | | | | Longitudinal | BC003965 |
| | | | | Increase | |
| | | | | Longitudinal | Micu2 |
| | | | | Increase | |
| | | | | Longitudinal | Cnot2 |
| | | | | Increase | |
| | | | | Longitudinal | Hspa4 |
| | | | | Increase | |
| | | | | Longitudinal | Ssh2 |
| | | | | Increase | |
| | | | | Longitudinal | Gsr |
| | | | | Increase | |
| | | | | Longitudinal | Dkc1 |
| | | | | Increase | |
| | | | | Longitudinal | Mbd2 |
| | | | | Increase | |
| | | | | Longitudinal | Brk1 |
| | | | | Increase | |
| | | | | Longitudinal | Tmem19 |
| | | | | Increase | |
| | | | | Longitudinal | Rhot2 |
| | | | | Increase | |
| | | | | Longitudinal | Arpc2 |
| | | | | Increase | |
| | | | | Longitudinal | Snx10 |
| | | | | Increase | |
| | | | | Longitudinal | Rps24 |
| | | | | Increase | |
| | | | | Longitudinal | Hdac8 |
| | | | | Increase | |
| | | | | Longitudinal | Uqcc2 |
| | | | | Increase | |
| | | | | Longitudinal | Smpd2 |
| | | | | Increase | |
| | | | | Longitudinal | Sugt1 |
| | | | | Increase | |
| | | | | Longitudinal | Lars |
| | | | | Increase | |
| | | | | Longitudinal | Aimp1 |
| | | | | Increase | |
| | | | | Longitudinal | Otud4 |
| | | | | Increase | |
| | | | | Longitudinal | Chmp4b |
| | | | | Increase | |
| | | | | Longitudinal | Fbxo31 |
| | | | | Increase | |
| | | | | Longitudinal | Dcun1d5 |
| | | | | Increase | |
| | | | | Longitudinal | Nop16 |
| | | | | Increase | |
| | | | | Longitudinal | Psmd7 |
| | | | | Increase | |
| | | | | Longitudinal | Tial1 |
| | | | | Increase | |
| | | | | Longitudinal | Atp6v1g1 |
| | | | | Increase | |

TABLE 4

| Geneset | Gene | Geneset | Gene |
|---|---|---|---|
| Development-Associated | Axin2 | Human HCC S1 | Gnai2 |
| Hepatocyte | | WNT Activ. | |
| Development-Associated | Krt8 | Human HCC S1 | S100a10 |
| Hepatocyte | | WNT Activ. | |
| Development-Associated | Lgr5 | Human HCC S1 | Aqp1 |
| Hepatocyte | | WNT Activ. | |
| Development-Associated | Cdkn1a | Human HCC S1 | Slc1a5 |
| Hepatocyte | | WNT Activ. | |
| Development-Associated | Hes1 | Human HCC S1 | Col3a1 |
| Hepatocyte | | WNT Activ. | |
| Development-Associated | Afp | Human HCC S1 | Dab2 |
| Hepatocyte | | WNT Activ. | |
| Development-Associated | Igfbp3 | Human HCC S1 | Anxa5 |
| Hepatocyte | | WNT Activ. | |
| Development-Associated | Cd24a | Human HCC S1 | Cd47 |
| Hepatocyte | | WNT Activ. | |
| Development-Associated | Ddr1 | Human HCC S1 | Ier3 |
| Hepatocyte | | WNT Activ. | |
| Development-Associated | Prom1 | Human HCC S1 | Arpc1b |
| Hepatocyte | | WNT Activ. | |
| Development-Associated | Jag1 | Human HCC S1 | Fcgr2b |
| Hepatocyte | | WNT Activ. | |
| Development-Associated | Sox9 | Human HCC S1 | Col4a1 |
| Hepatocyte | | WNT Activ. | |
| Development-Associated | Epcam | Human HCC S1 | M6pr |
| Hepatocyte | | WNT Activ. | |
| | | Human HCC S1 | Iqgap1 |
| | | WNT Activ. | |
| | | Human HCC S1 | Tmsb4x |
| | | WNT Activ. | |
| | | Human HCC S1 | Lgals1 |
| | | WNT Activ. | |
| | | Human HCC S1 | Aldoa |
| | | WNT Activ. | |
| | | Human HCC S1 | Gstp2 |
| | | WNT Activ. | |
| | | Human HCC S1 | Hexa |
| | | WNT Activ. | |
| | | Human HCC S1 | Itpr3 |
| | | WNT Activ. | |
| | | Human HCC S1 | H2-Aa |
| | | WNT Activ. | |
| | | Human HCC S1 | Tcf4 |
| | | WNT Activ. | |
| | | Human HCC S1 | Lgals3bp |
| | | WNT Activ. | |
| | | Human HCC S1 | H2-Ab1 |
| | | WNT Activ. | |
| | | Human HCC S1 | Traf5 |
| | | WNT Activ. | |
| | | Human HCC S1 | Arpc2 |
| | | WNT Activ. | |
| | | Human HCC S1 | Col1a2 |
| | | WNT Activ. | |
| | | Human HCC S1 | Mthfd2 |
| | | WNT Activ. | |
| | | Human HCC S1 | Cd151 |
| | | WNT Activ. | |
| | | Human HCC S1 | Cd74 |
| | | WNT Activ. | |
| | | Human HCC S1 | Crip2 |
| | | WNT Activ. | |
| | | Human HCC S1 | Dctn2 |
| | | WNT Activ. | |
| | | Human HCC S1 | Grn |
| | | WNT Activ. | |
| | | Human HCC S1 | C1qb |
| | | WNT Activ. | |
| | | Human HCC S1 | Atp6v0b |
| | | WNT Activ. | |
| | | Human HCC S1 | Col4a2 |
| | | WNT Activ. | |
| | | Human HCC S1 | Ralb |
| | | WNT Activ. | |
| | | Human HCC S1 | Ccn2 |
| | | WNT Activ. | |
| | | Human HCC S1 | Celf2 |
| | | WNT Activ. | |
| | | Human HCC S1 | Dgkz |
| | | WNT Activ. | |
| | | Human HCC S1 | Ddr1 |
| | | WNT Activ. | |
| | | Human HCC S1 | Srgn |
| | | WNT Activ. | |
| | | Human HCC S1 | Slc7a5 |
| | | WNT Activ. | |
| | | Human HCC S1 | Rhoa |
| | | WNT Activ. | |
| | | Human HCC S1 | Lgmn |
| | | WNT Activ. | |
| | | Human HCC S1 | Gpnmb |
| | | WNT Activ. | |
| | | Human HCC S1 | Ctss |
| | | WNT Activ. | |
| | | Human HCC S1 | Tuba4a |
| | | WNT Activ. | |
| | | Human HCC S1 | Atp6v1b2 |
| | | WNT Activ. | |
| | | Human HCC S1 | Slc2a1 |
| | | WNT Activ. | |
| | | Human HCC S1 | Lhfpl2 |
| | | WNT Activ. | |
| | | Human HCC S1 | S100a11 |
| | | WNT Activ. | |
| | | Human HCC S1 | Gns |
| | | WNT Activ. | |
| | | Human HCC S1 | Flna |
| | | WNT Activ. | |
| | | Human HCC S1 | Pea15a |
| | | WNT Activ. | |
| | | Human HCC S1 | Trip10 |
| | | WNT Activ. | |
| | | Human HCC S1 | Psmd2 |
| | | WNT Activ. | |
| | | Human HCC S1 | Oaz1 |
| | | WNT Activ. | |
| | | Human HCC S1 | Atp1b3 |
| | | WNT Activ. | |
| | | Human HCC S1 | Tspan3 |
| | | WNT Activ. | |

### Example 3: Responses to Metabolic Stress Extend to Human MASLD Progression, Exhibit Extreme Manifestations in Cancer, and are Prognostic for Cancer Survival

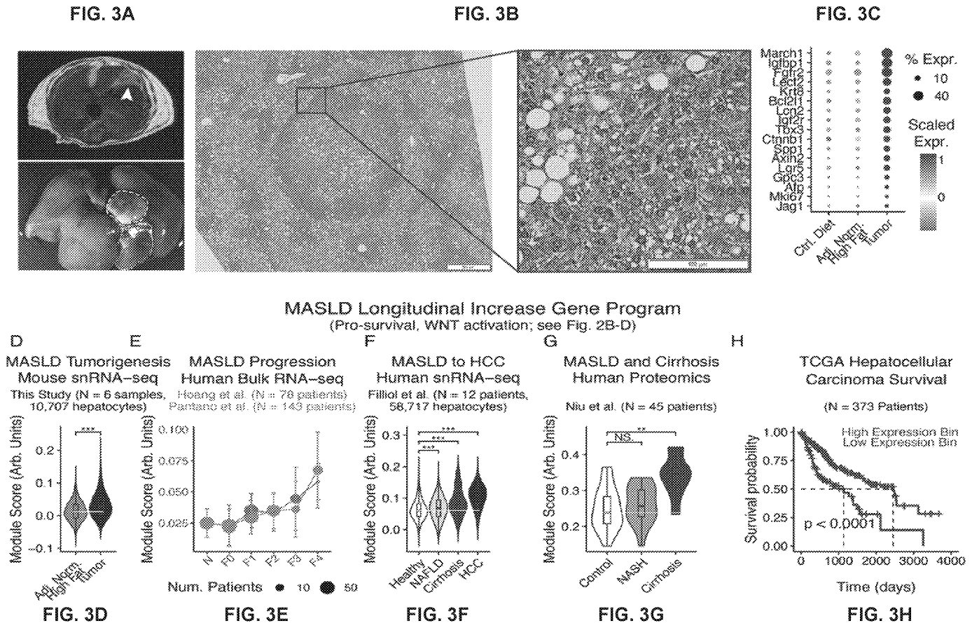

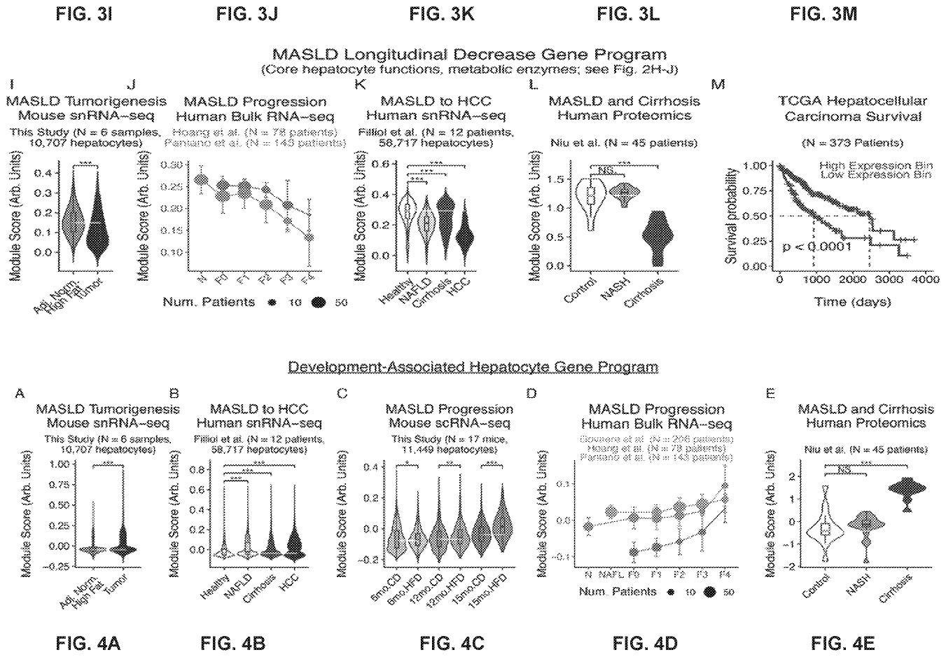

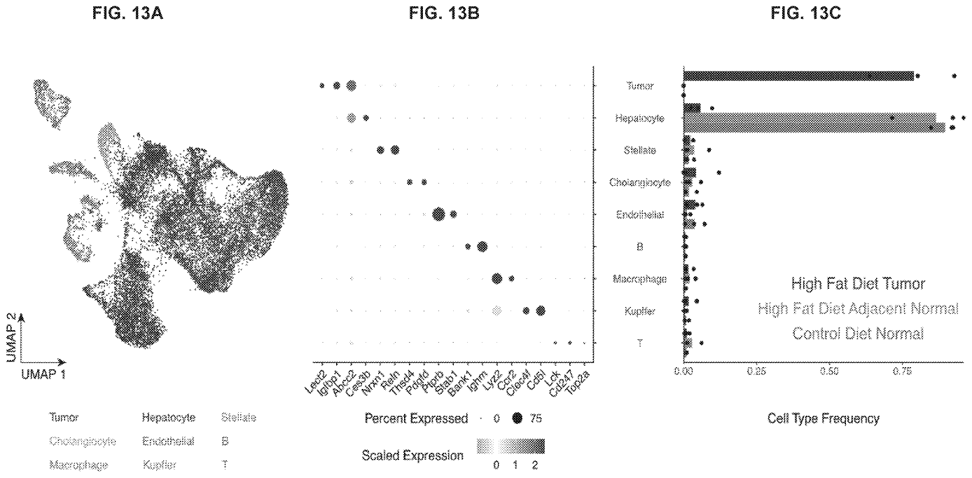

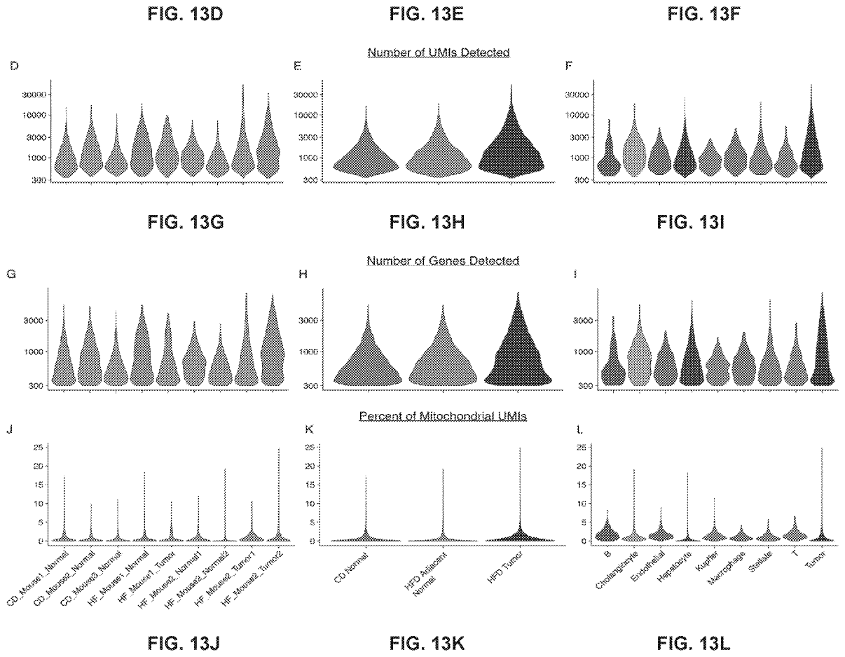

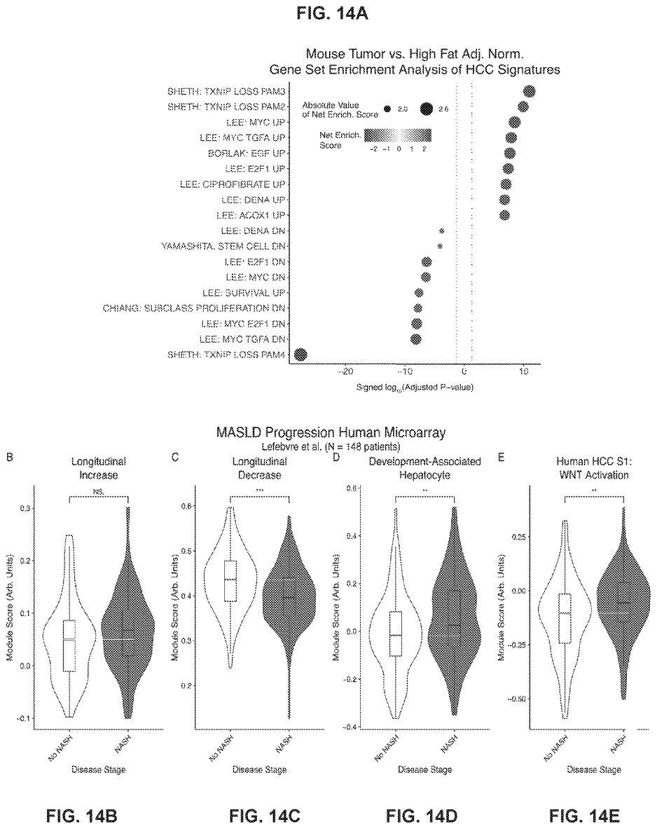

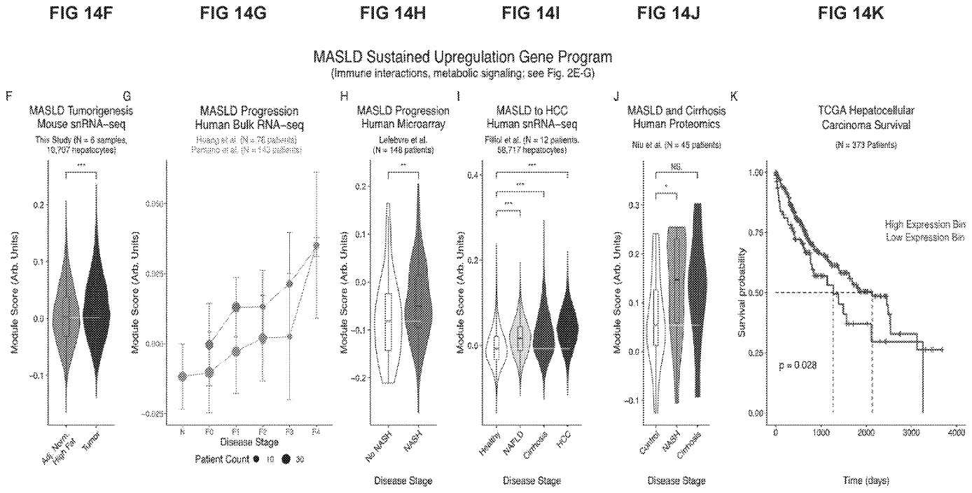

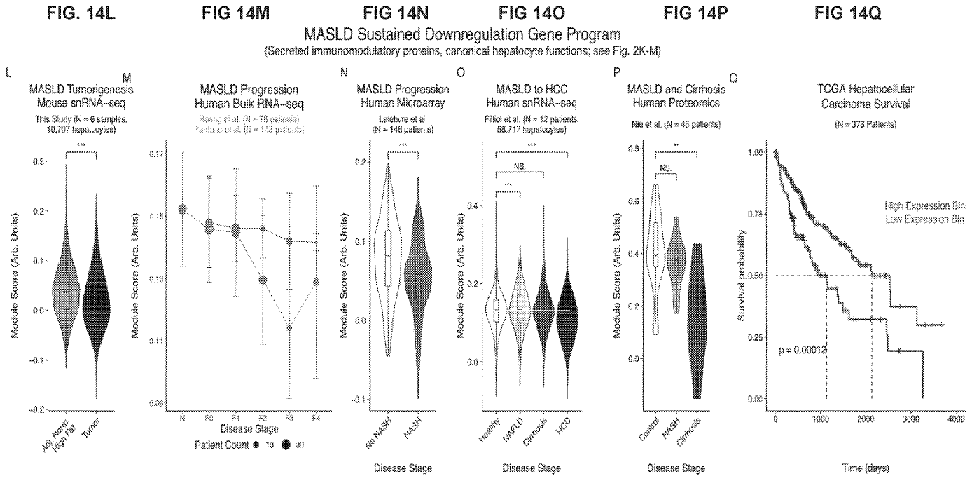

It was next examined whether hepatocytes' chronic stress adaptations and tradeoffs connected to tumor phenotypes, predicted long-term disease outcomes, and extended to human patient cohorts. MRI, gross imaging, and histologic evaluation supported liver tumors' classification as HCC, with preserved liver architecture but also pleomorphism, prominent nucleoli, and hyperchromasia (FIG. 3B). Through snRNA-seq, tumors in the mouse model recapitulated human HCC markers, such as WNT and Notch pathway members (e.g., Axin2, Tbx3, Ctnnb1, Lgr5, Jag1), IGF signaling (e.g., Igfbp1, Igf2r), HCC diagnostic biomarkers (e.g., Afp, Gpc3), and proliferation (e.g., Mki67) (FIGS. 3B-3C, 13). More holistic analyses revealed enrichment and expected directionality of liver cancer marker sets defined in previous studies (FIG. 14A).

With these benchmarks on tumor phenotypes, the issue of whether and how stress adaptation tradeoffs aligned with tumorigenesis-associated alterations was studied. Tumor cells exhibited elevated expression of the Longitudinal Increase program relative to adjacent, non-transformed hepatocytes (FIG. 3D). External human cohorts exhibited a similar pattern, with the Longitudinal Increase program positively associated with human disease severity across microarray, bulk RNA-seq, and snRNA-seq readouts (FIGS. 3E-F; FIG. 14B). Protein-level abundances of the Longitudinal Increase program likewise increased across disease progression in human liver tissue proteomics (FIG. 3G). Finally, it was found that the Longitudinal Increase program prognostically stratified HCC patients' survival, with high expression predicting worsened outcomes (FIG. 3H). Thus, the same pathways activated as adaptations to metabolic stress in non-transformed hepatocytes (e.g., pro-survival effectors, WNT activation) are further upregulated with tumorigenesis, so that tumor cells exhibit a heightened manifestation and extension of metabolic overload-induced adaptations.

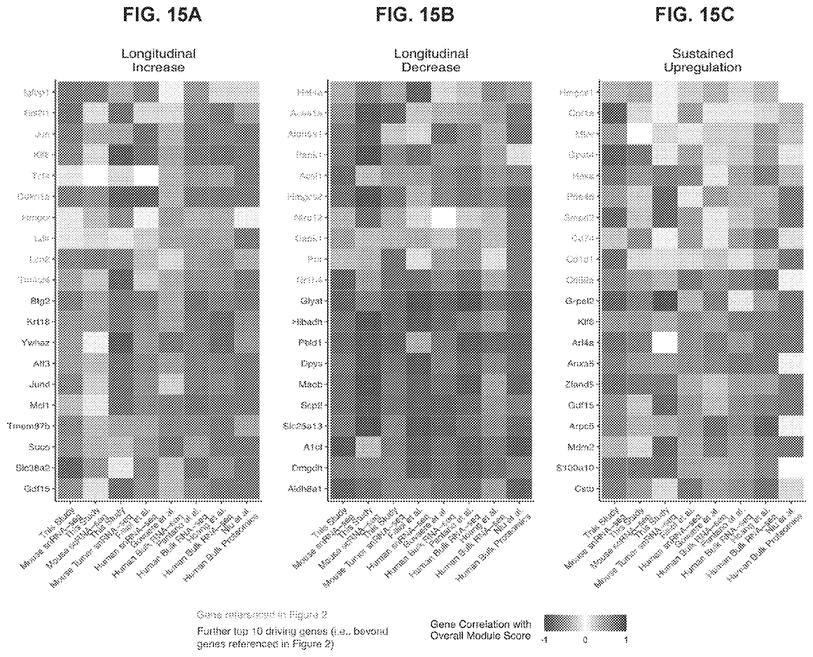

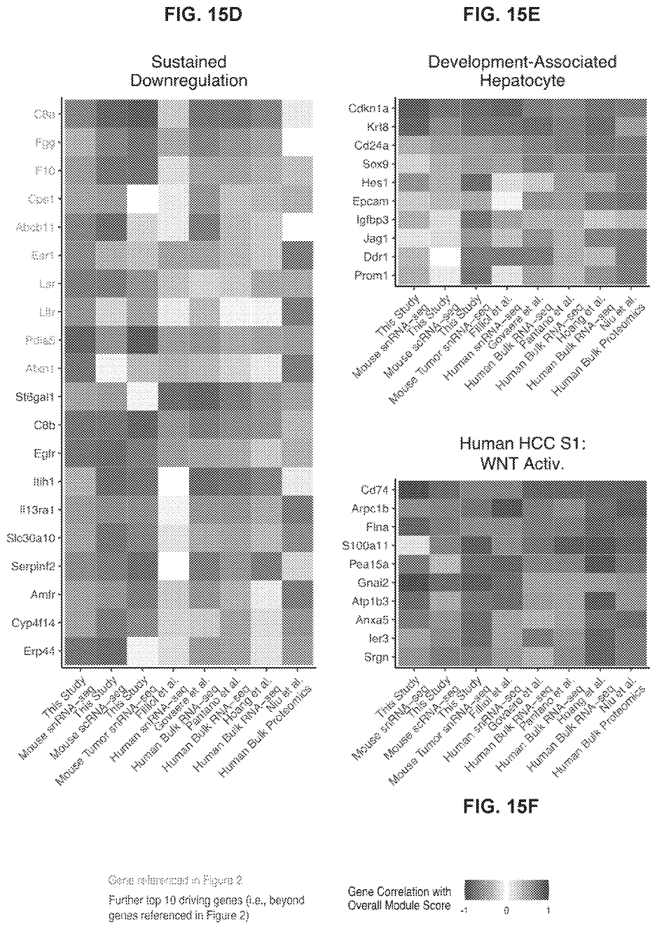

The Longitudinal Decrease, Sustained Upregulation, and Sustained Downregulation programs also significantly associated with disease severity, tumor phenotypes, and patient survival outcomes (FIGS. 31-3M; FIGS. 14C-14Q). As internal consistency, the Longitudinal Increase and Sustained Upregulation programs displayed opposite directionalities as did the Longitudinal Decrease and Sustained Downregulation programs. Additionally, across studies, measurement modalities, and species, aggregate module scores were driven by similar underlying genes, with strong positive covariance between individual genes and overall module scores (FIGS. 15A-15D).

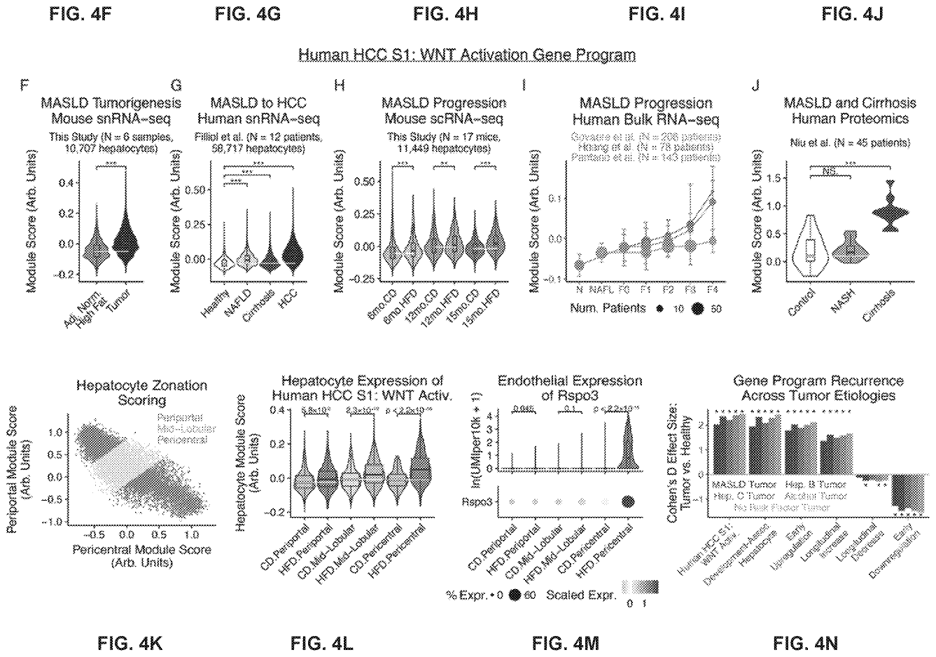

Complementing connections from non-transformed hepatocytes' early stress adaptations to later tumor phenotypes and outcomes, whether HCC-associated signatures were observed early in MASLD before overt tumorigenesis was investigated. Upon examining signatures of: 1) cancer-linked chemical and genetic perturbations, 2) HCC mutational subtypes, and 3) liver development and regeneration, it was found that hepatocytes early in MASLD take on gene expression patterns reminiscent of earlier developmental stages and the HCC S1 subclass. Development-associated expression patterns (driven by genes including Krt8, Sox9, and Cd24a, FIG. 15E) were increased not only in HCC, but even at early disease stages in the MASLD progression in non-transformed hepatocytes across species and measurement modalities (FIGS. 4A-4E). Likewise, marker genes of the human HCC S1 subclass, characterized by aberrant WNT activation, were elevated in hepatocytes across species at early MASLD stages and further elevated with tumorigenesis (FIGS. 4F-4J, FIG. 15F). Contextualizing transcriptional shifts, β-catenin regulates WNT signaling activation and is a commonly mutated HCC driver gene, suggesting pathway-level convergence between stress-induced transcriptional alterations and literature-established genomic drivers.

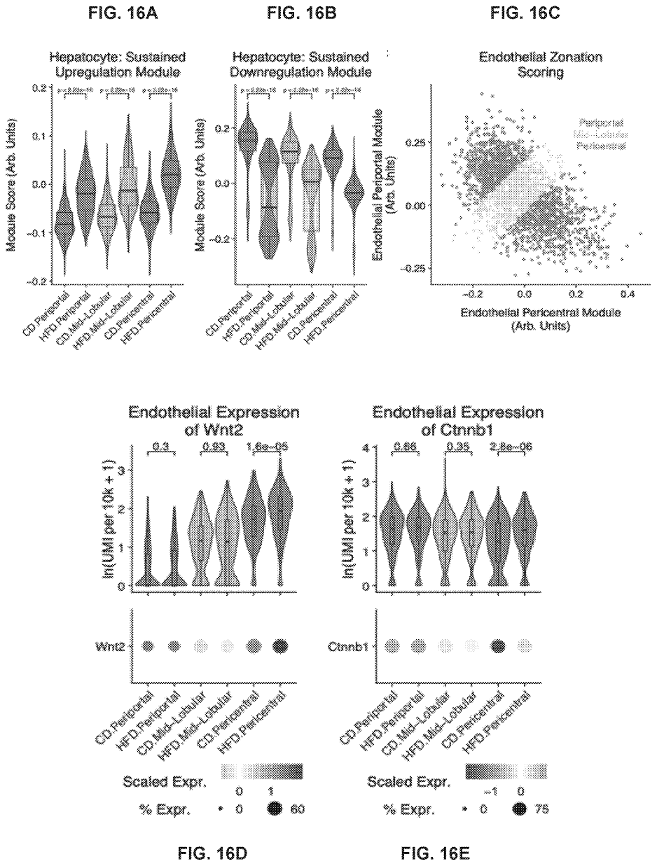

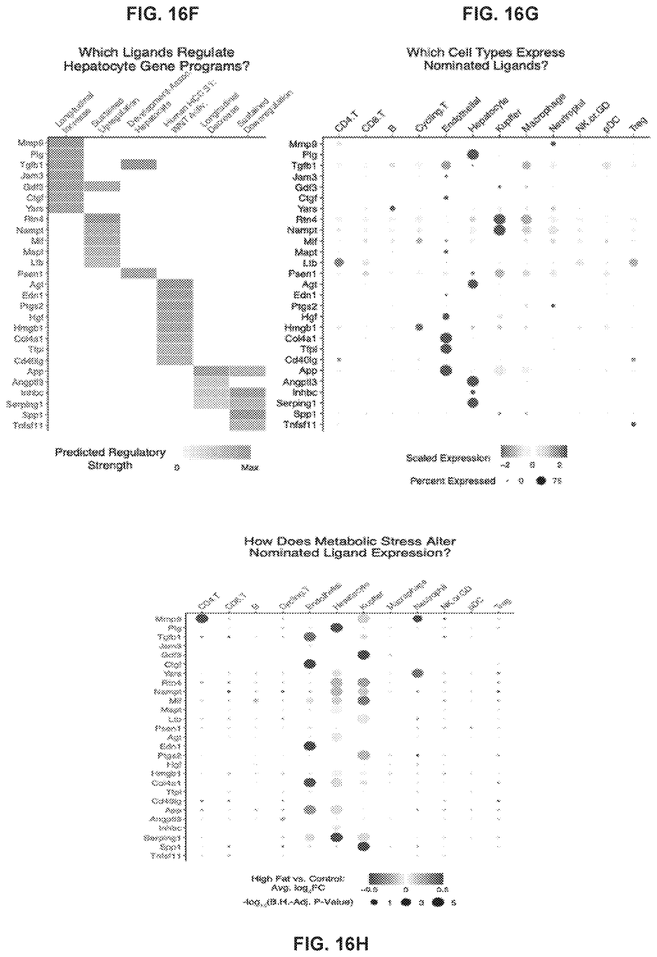

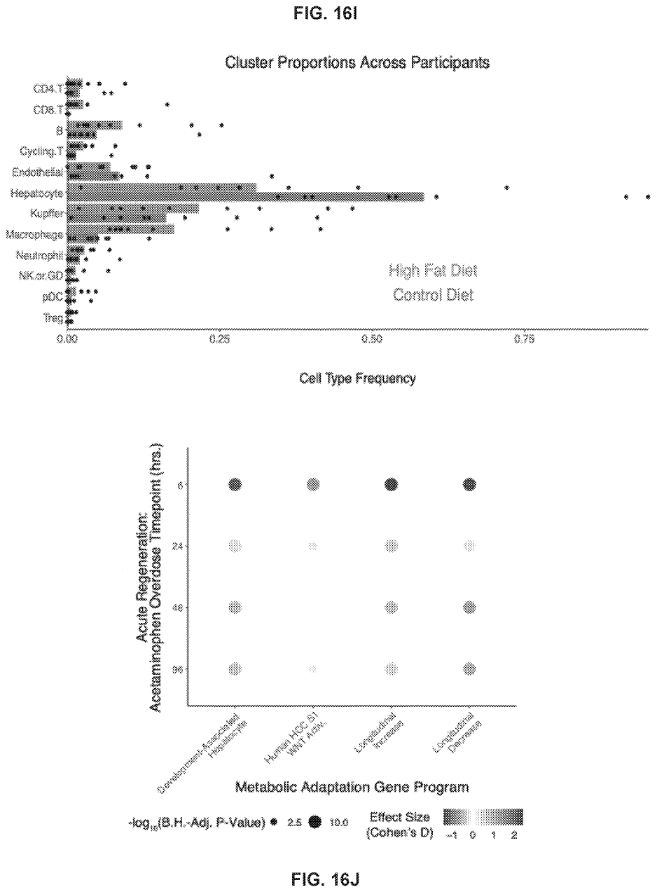

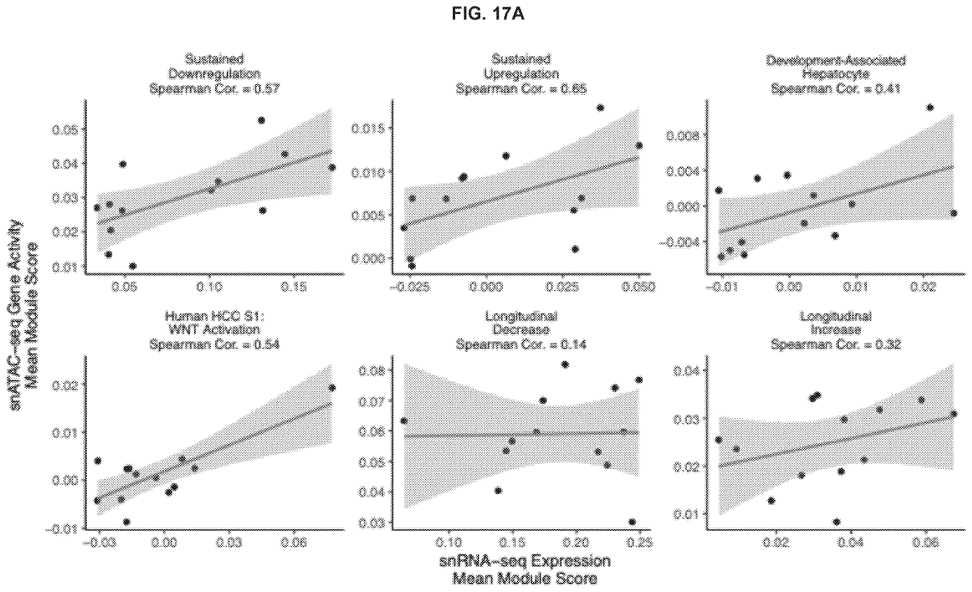

Towards broader spatial context, hepatocytes' spatial location was inferred via periportal-vs-pericentral markers (FIG. 4K). Pre-malignant pericentral hepatocytes exhibited larger increases in the WNT activation-associated HCC S1 signature than did periportal hepatocytes (FIG. 4L). Pericentral endothelial cells are a key source of secreted WNT ligands. Upon similarly inferring endothelial cell zonation, it was found that pericentral endothelial cells (but not periportal or mid-lobular endothelial cells) upregulate expression of Rspo3, Wnt2, and Ctnnb1 with chronic metabolic stress, potentially aligning with spatially-structured signaling circuits (FIGS. 4M, 16A-16J; see Example 9 for activation of hepatocytes' chronic stress adaptation programs during acute regeneration and analyses of broader intercellular interactions). To distinguish etiology-specific vs. generalizable adaptation mechanisms, it was found that hepatocytes' metabolic adaptation programs were consistently dysregulated across HCC tumors linked to metabolic disorders (i.e., MASLD, alcohol-related liver disease), viral infection (i.e., hepatitis B and hepatitis C virus), and no known risk factors (FIG. 4N). Internally-consistent directionalities across HCC etiologies supports the generalizability of these axes of hepatocyte functional adaptations in wide-ranging disease microenvironments.

Thus, metabolic stress-induced adaptations in non-transformed hepatocytes were linked to later tumor phenotypes: extensions to human cohorts, similarities to later tumor states, and predictive power for disease severity and HCC patient survival. The directionality of effects on human HCC survival helps propose interpretations of these axes of hepatocyte adaptation: while elevated processes could plausibly be linked to improved survival of individual cells, they incur longer-term repercussions through reductions in hepatocytes' identity-defining features and tissue-scale functions, as well as early activation of pathways eventually contributing to tumorigenesis and worsened survival at later disease stages.

### Example 4: Epigenetic Dysregulation and WNT Pathway Priming Under Chronic Metabolic Stress

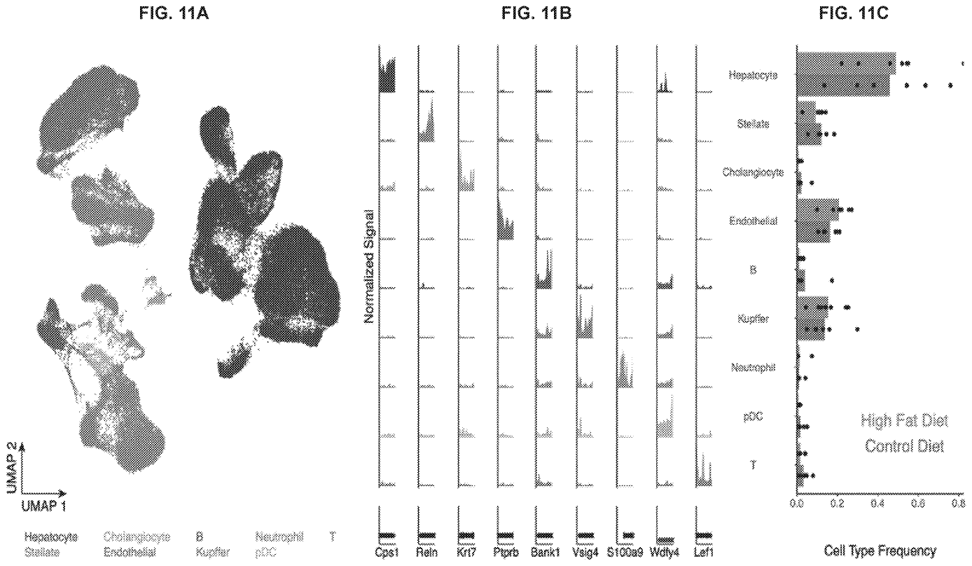

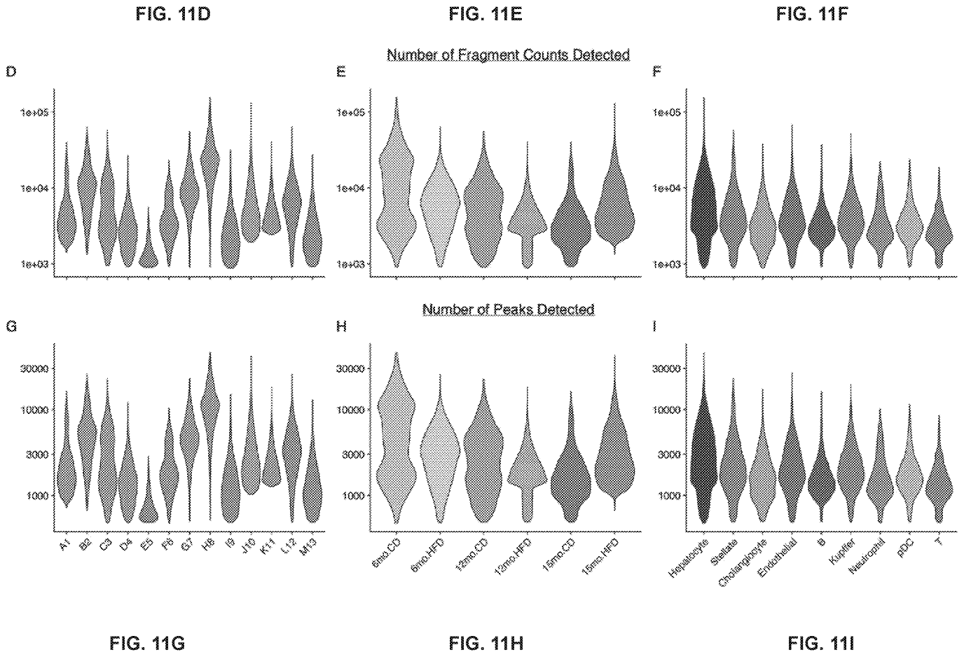

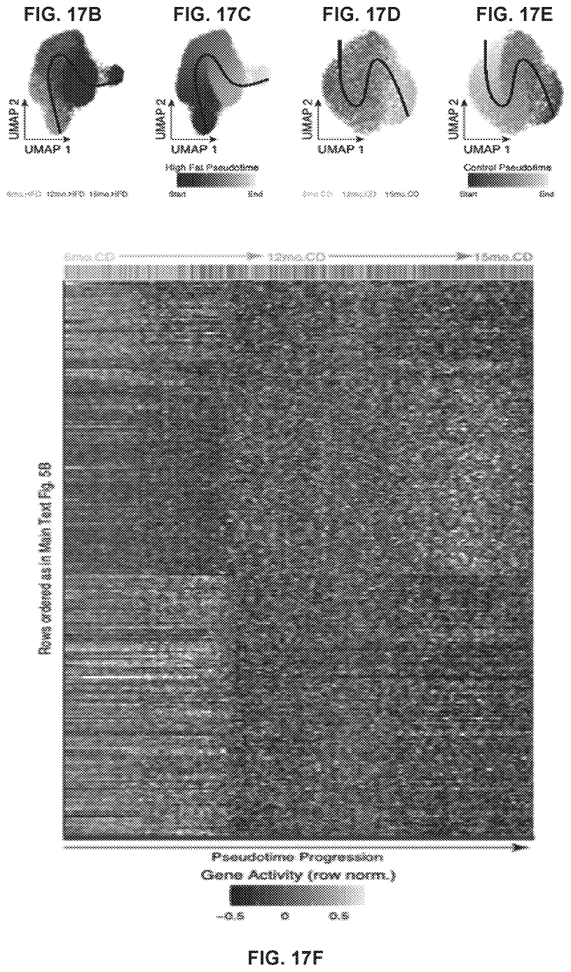

Having defined the axes of hepatocytes' longitudinal adaptations to metabolic stress, mechanistic insight into the epigenetic landscape and cell-intrinsic regulatory factors shaping hepatocyte decision-making under stressful disease microenvironments was sought (FIG. 11). As reinforcement and validation of hepatocytes' metabolic adaptation programs across genomic regulatory layers, concordant correlations were found between their transcriptional expression and epigenetic accessibility (FIG. 17A).

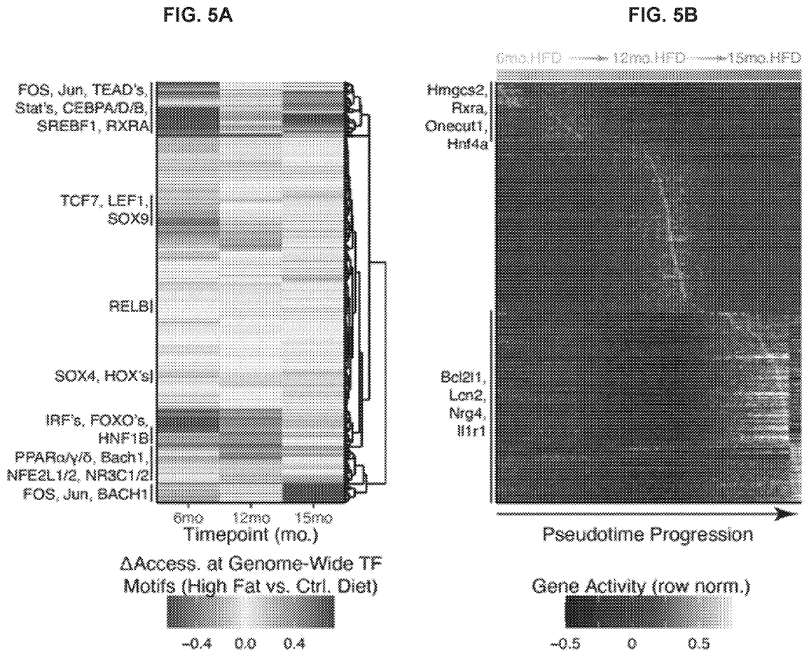

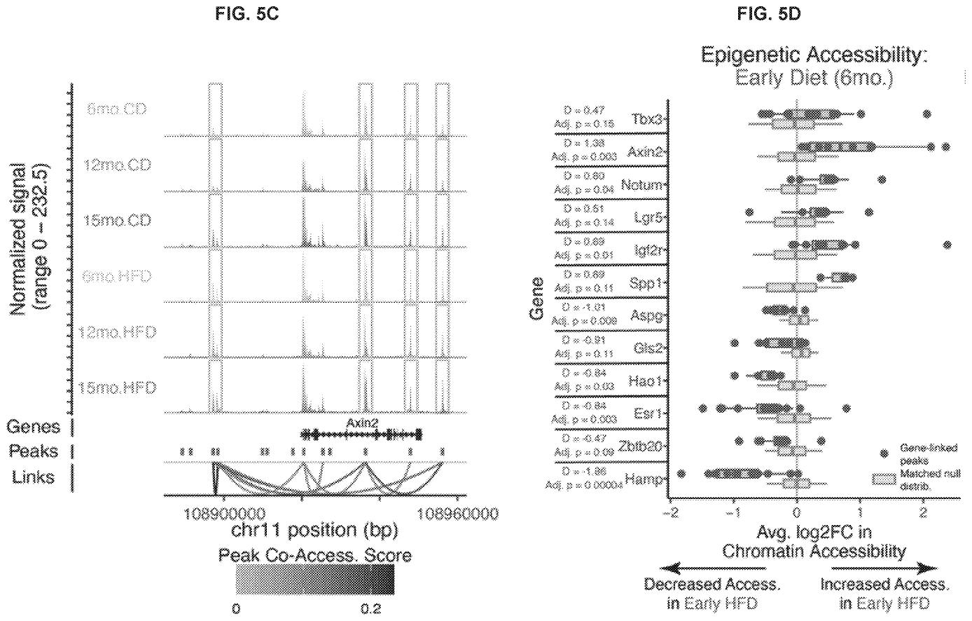

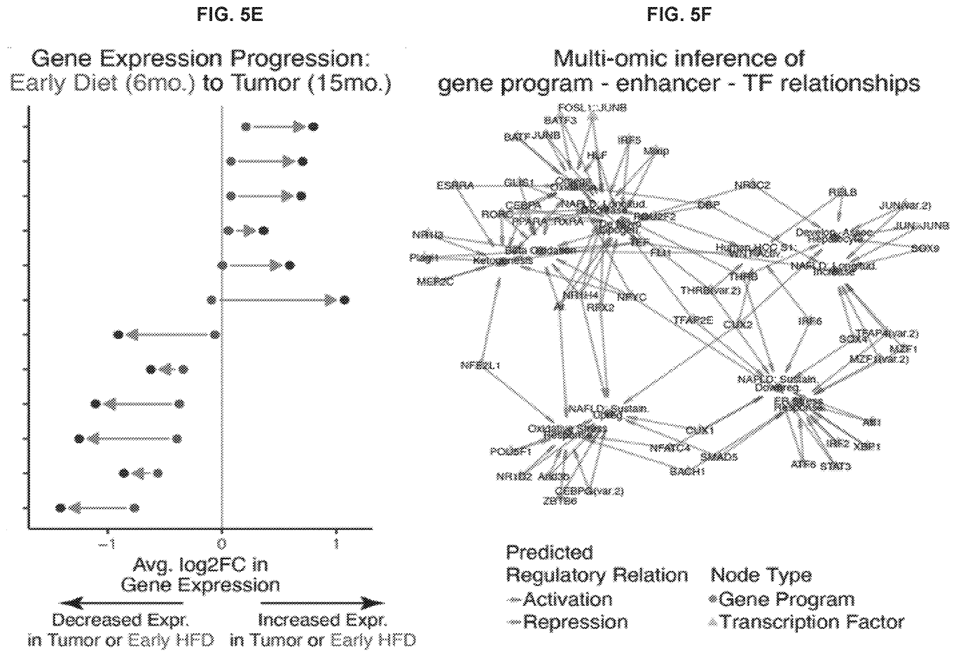

To identify transcription factors (TFs) altered by metabolic stress in hepatocytes, genome-wide accessibility of chromatin peaks containing TFs' binding motifs in the snATAC-seq dataset were examined (FIG. 5A). Progressive increases in motif accessibility for members of the AP-1 complex were observed, which play roles in responses to cellular stressors as well as mediate long-lasting epigenetic rewiring and tissue memory of inflammation. Increased TEAD motif accessibility suggests involvement of the Hippo pathway, which regulates liver regeneration and development. Reinforcing the inference of early activation of WNT pathway and development-associated programs, increased motif accessibility for WNT-associated TFs (e.g., TCF7, LEF1) and TFs active during liver development (e.g., SOX4, SOX9) was observed. As TFs with increased motif accessibility across both early and long-term metabolic stress, PPAR members (regulating liver metabolic pathways and serving as a leading MASLD drug target, but also driving stemness and regeneration phenotypes in intestinal stem cells), RXRA-associated nuclear receptor TF complexes, and NFE2L1 (a key regulator of cellular responses to oxidative stress).

Towards higher-resolution insights into temporally-varying chromatin states, pseudotime analyses were conducted to order hepatocytes according to smooth gradients in chromatin accessibility, prioritizing gene loci dynamically altered by chronic metabolic stress (FIGS. 5B, 17B-17E). Chromatin trajectories reinforced the multi-omic, cross-species analyses of hepatocyte adaptations: members of the Longitudinal Decrease program including Hmgcs2, Acsl1, Scp2, Hnf4a, and Rxra exhibited maximal chromatin accessibility early in the high fat diet pseudotime progression, whereas members of the Longitudinal Increase program like Lcn2, Bcl2l1, and Il1r1 peaked towards the pseudotime terminus. Progressive decreases in chromatin accessibility at Hnf4a's gene locus are notable for this transcription factor's important role as a master regulator of hepatocyte identity, which aligns with its transcriptional and proteomic downregulation, decreases of canonical hepatocyte functions, and increases in developmental marker expression. These (pseudo) temporal patterns were not observed in hepatocytes from control diet mice, supporting distinctive trajectories of stress-induced chromatin remodeling (FIGS. 17B-17F).

Researchers additionally sought to identify genes where epigenetic alterations preceded and presaged transcriptomic shifts. These may indicate stress-induced epigenetic priming: chromatin remodeling establishing accessible epigenetic landscapes prior to transcriptional alterations, thereby priming cells for later activation and (dys) function. Co-accessibility between intergenic chromatin peaks and promoters or gene bodies to create peak-gene linkages was leveraged, capturing enhancer-gene regulatory interactions despite potentially large genomic distances. The regeneration-upregulated, WNT/β-catenin target Axin2 provides an example, with several distal chromatin regions co-accessible with Axin2's promoter/gene body and strongly elevated in accessibility with HFD across timepoints (FIG. 5C). More broadly, a variety of WNT-associated (e.g., Tbx3, Axin2, Lgr5, Notum) and HCC-linked (e.g., Spp1, Igf2r) genes exhibited increased epigenetic accessibility but only small changes in transcription at the earliest timepoint (6 months; FIGS. 5D-5E). However, these genes were strongly upregulated after tumorigenesis months later (15 months; FIG. 5E). Genes with the opposite directionality (i.e., early decreases in chromatin accessibility preceding more extreme transcriptional downregulation with longer-term metabolic stress) included: 1) metabolic enzymes and secreted proteins (e.g., Aspg, Hao1, Hamp), 2) suppressors of HCC and fetal hepatocyte-associated phenotypes (Gls2 and Zbtb20, respectively), and, 3) Esr1 as a receptor for MASLD-protective estrogen signaling (FIGS. 5E-5F).

Thus, paralleling hepatocytes' transcriptomic and proteomic stress adaptations, discovery of early WNT- and HCC-linked chromatin changes that foreshadow transcriptional shifts suggests that even early exposure to metabolic stress may establish a permissive chromatin landscape that contributes to later activation of regeneration-, development-, and cancer-associated pathways.

### Example 5: MATCHA Prioritizes Causal Transcription Factors Shaping MASLD-Associated Phenotypes

Computational nomination of TFs driving functionally-important gene programs (e.g., hepatocytes' disease-linked stress adaptation programs) remains an unsolved, open problem (see Example 10 for contextualization of prior work). MATCHA (Multiomic Ascertainment of Transcriptional Causality via Hierarchical Association), a computational framework to map user-specified gene programs (e.g., arbitrary biological processes, disease (mal) adaptations, etc.) to distal enhancers and program-specific TF activities (see Methods) was therefore developed. In brief, MATCHA links gene programs to cell-type-specific distal enhancers by identifying chromatin regions co-accessible with the gene program's promoters or gene bodies. MATCHA then prioritizes causal regulators by determining TF motifs whose accessibility at program-coaccessible enhancers likewise covaries with program transcriptional expression. MATCHA further optionally incorporates: 1) concordance across datasets towards robust regulatory inference (e.g., across species, single-cell vs. bulk measurements, etc.), or, 2) identification of TFs co-regulating multiple gene programs. MATCHA therefore enables prioritization of TFs driving arbitrary gene programs while also modeling context-dependent functions via cell type- and tissue-specific gene regulatory landscapes.

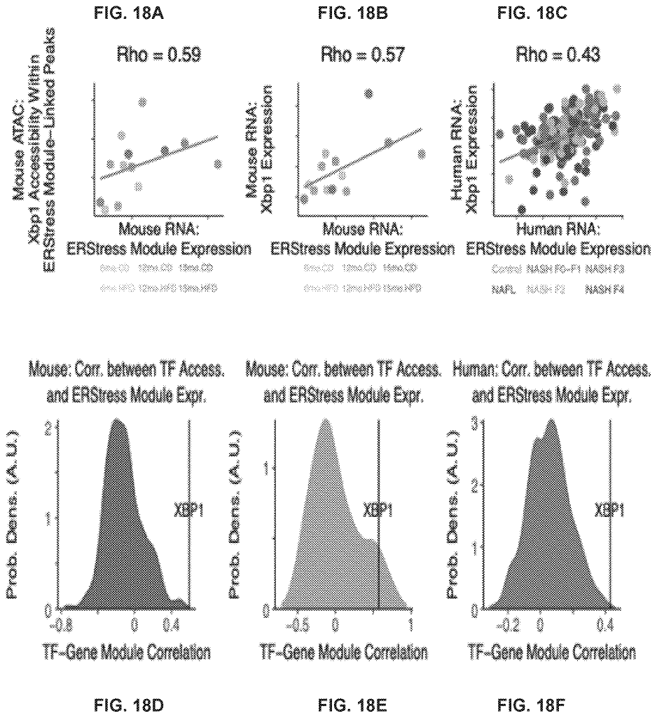

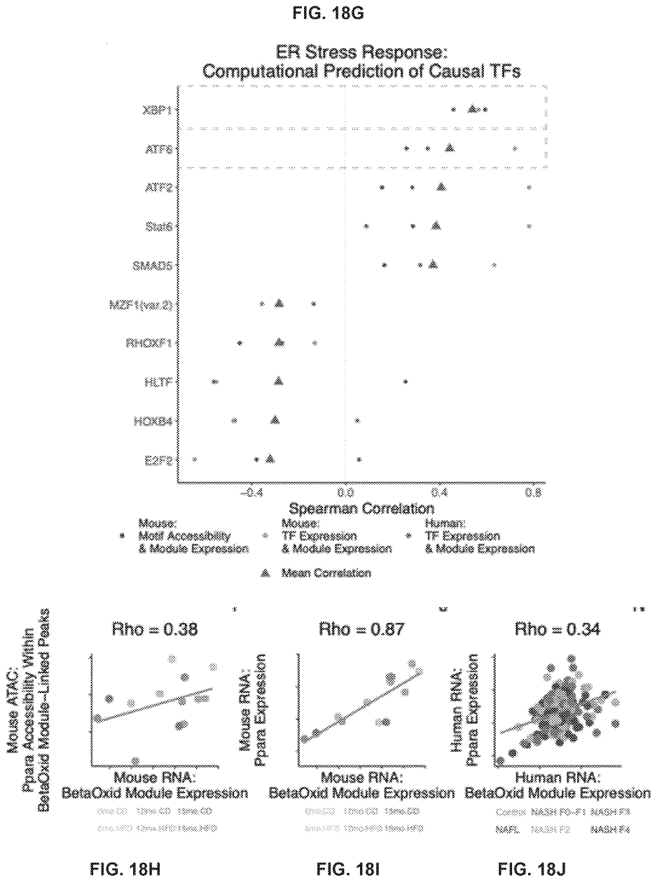

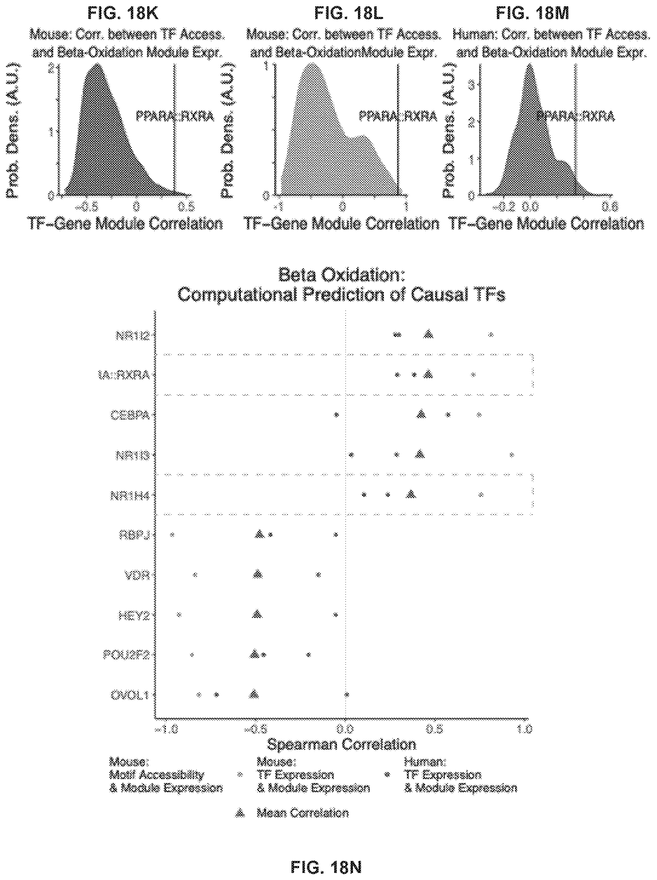

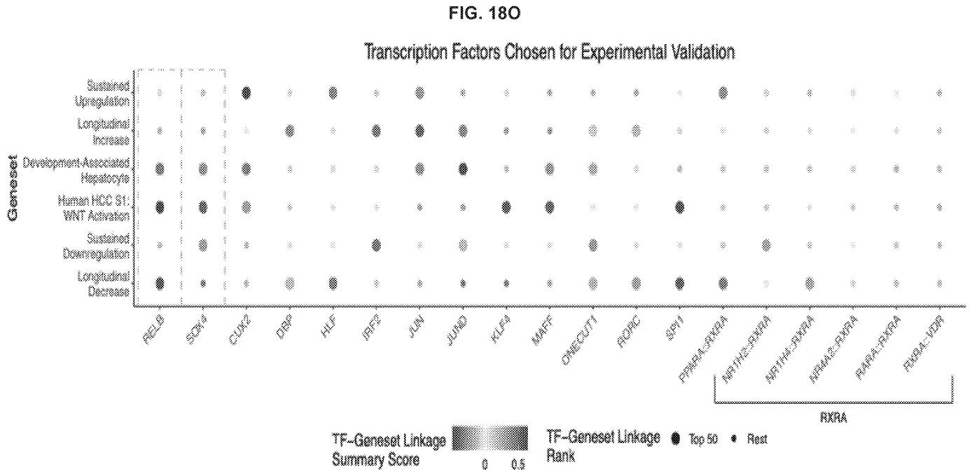

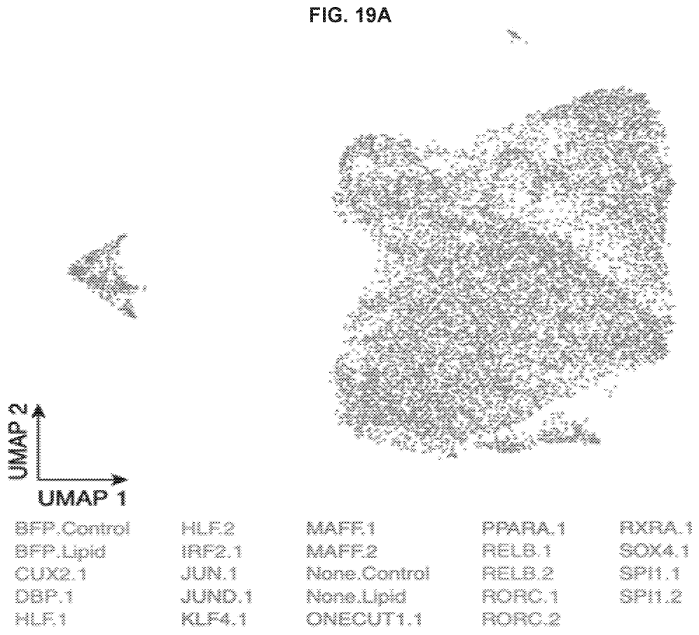

As proof-of-concept, two metabolic-stress-relevant processes with known driver TFs: ER stress response and beta-oxidation (defined externally through GO:BP) were examined. MATCHA recovered ground-truth causal TFs for these external test cases: the top two TFs prioritized for GO:BP ER stress response genes were XBP1 and ATF6 (i.e., two of three well-established master regulatory TFs) (FIGS. 18A-18G). The top 5 TFs for GO:BP beta-oxidation included FXR/NR1H4 and PPARα (whose agonists have advanced to Phase III clinical trials for MASLD), and NR1I2, CEBPA, and NR113 (all with preclinical evidence for regulation of liver metabolism and lipid accumulation; FIGS. 18H-18N).

To identify core TFs mediating hepatocytes' longitudinal stress adaptations and early induction of development-associated and HCC-linked cell states, MATCHA was applied to construct a bipartite network of regulatory relationships between gene programs and TFs, supported by epigenetic and transcriptional evidence (FIG. 5F). The network successfully recapitulated known ground truths, but also nominated targets for experimental validation. In addition to previously-discussed well-known drivers of ER stress (i.e., XBP1, ATF6) and beta-oxidation (i.e., PPARα, NR1H4/FXR), the network captured hepatocyte developmental regulation (i.e., SOX9 driving development states), hormonal influences on metabolism (i.e., androgen receptor driving beta-oxidation), and stress response mediators (i.e., NFE2L1/NRF1 driving oxidative stress response). Regulatory links between THRB motifs, activation of hepatocyte identity-linked Longitudinal Decrease and Sustained Downregulation programs, and repression of development-associated and HCC S1 WNT programs were inferred; the THRB agonist resmetirom is currently undergoing the Phase III MAESTRO-NASH trial. Researchers chose 15 TFs (20 isoforms) to prioritize for experimental validation (FIG. 18O).

### Example 6: RELB and SOX4 Mediate Wide-Ranging MASLD Adaptation and Tumor-Associated Functional Phenotypes

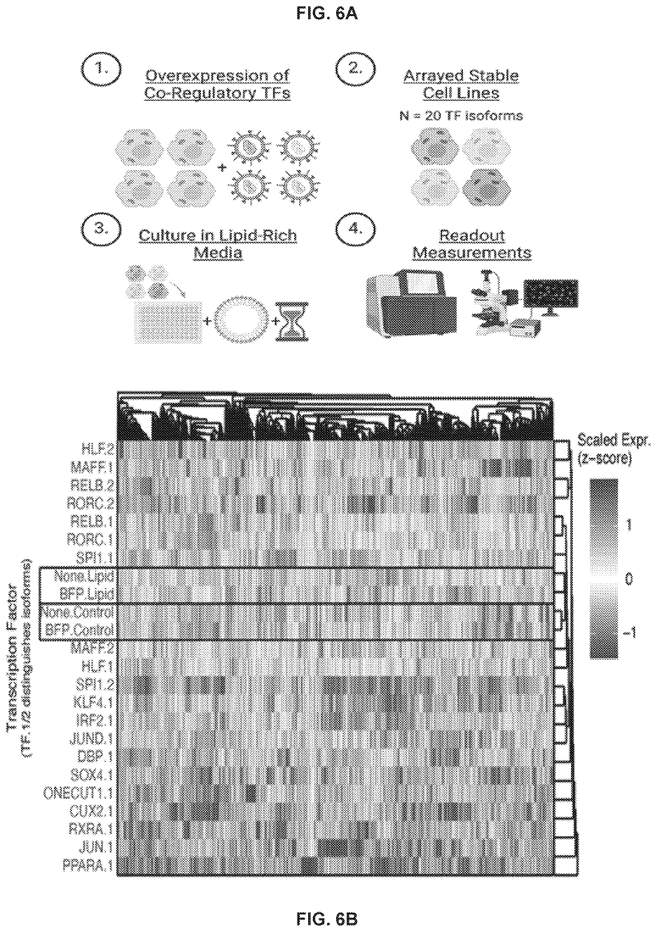

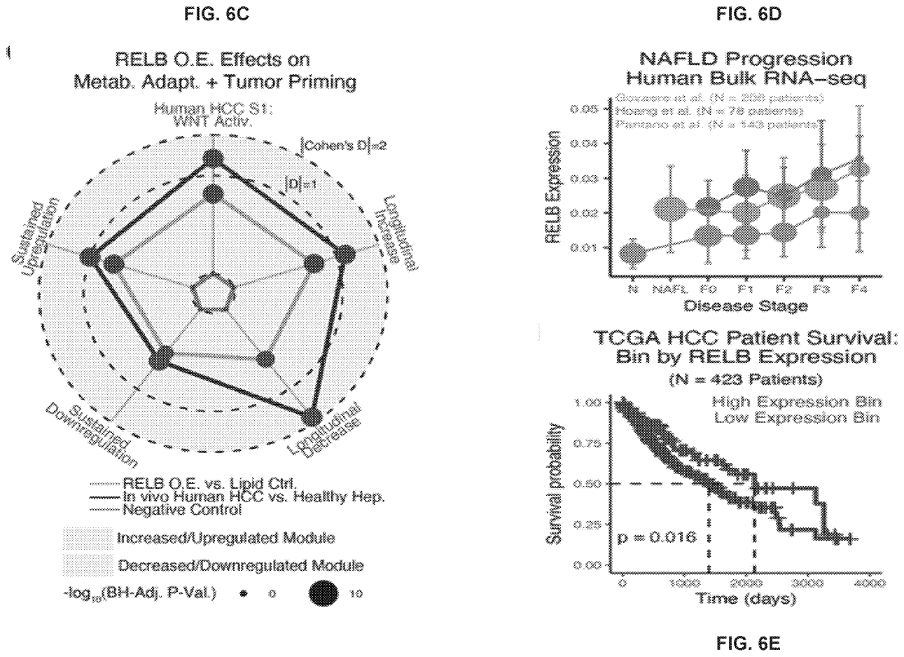

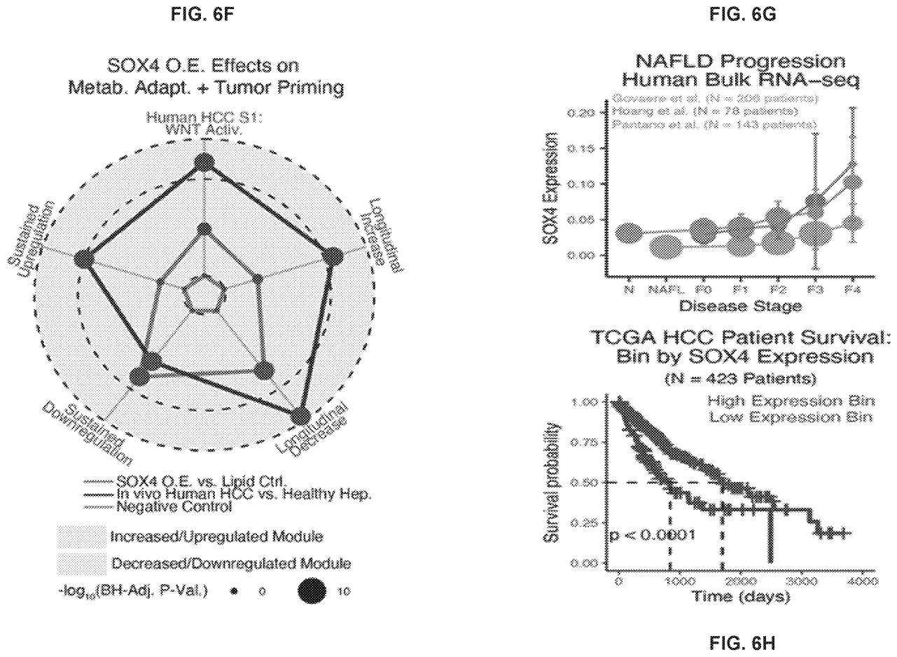

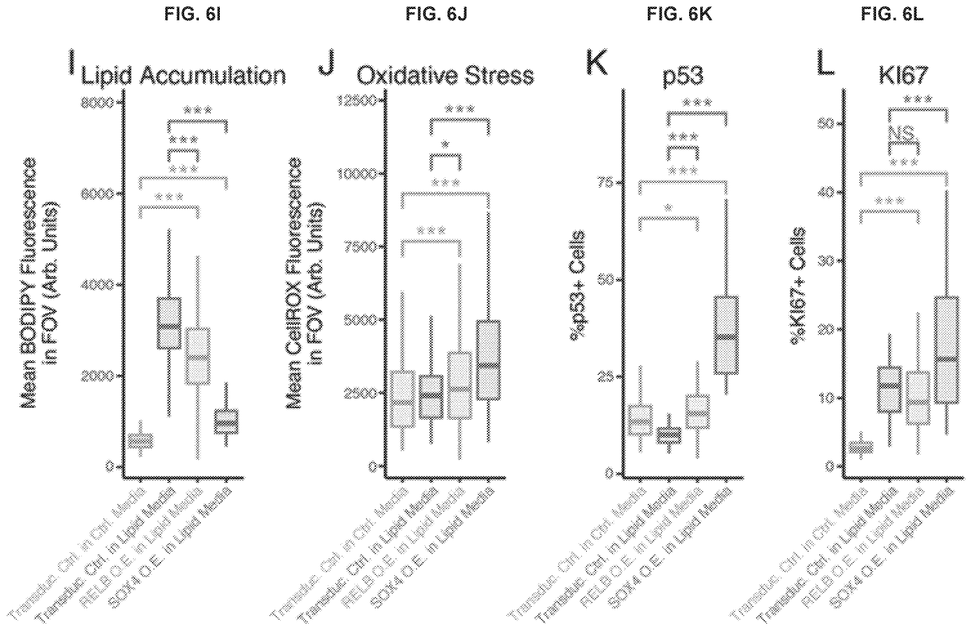

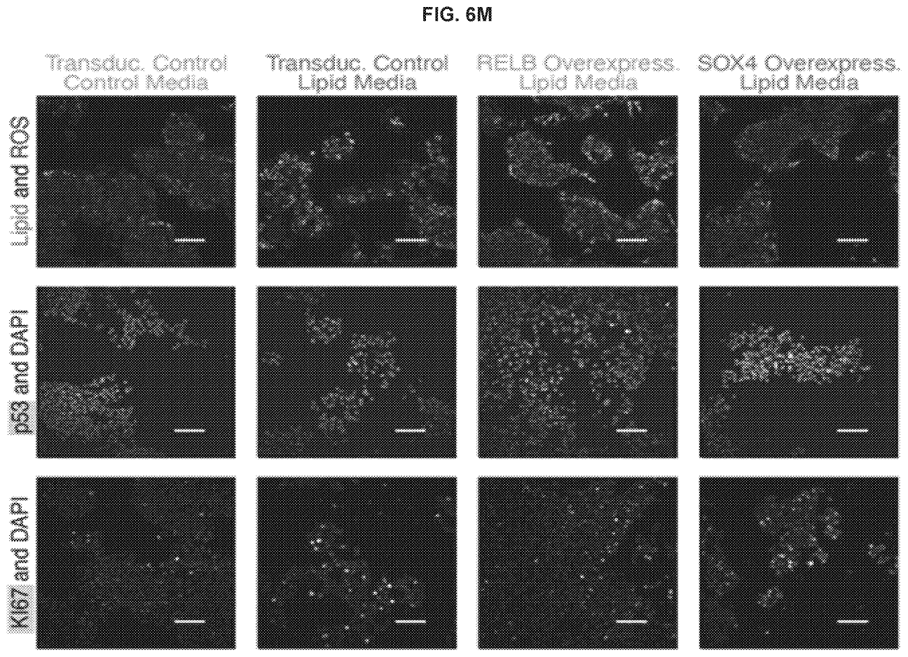

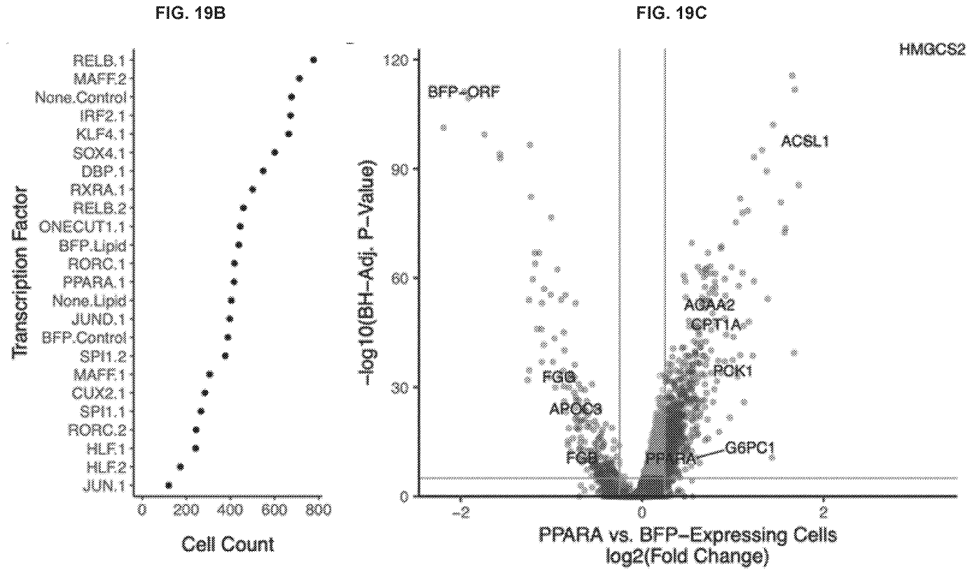

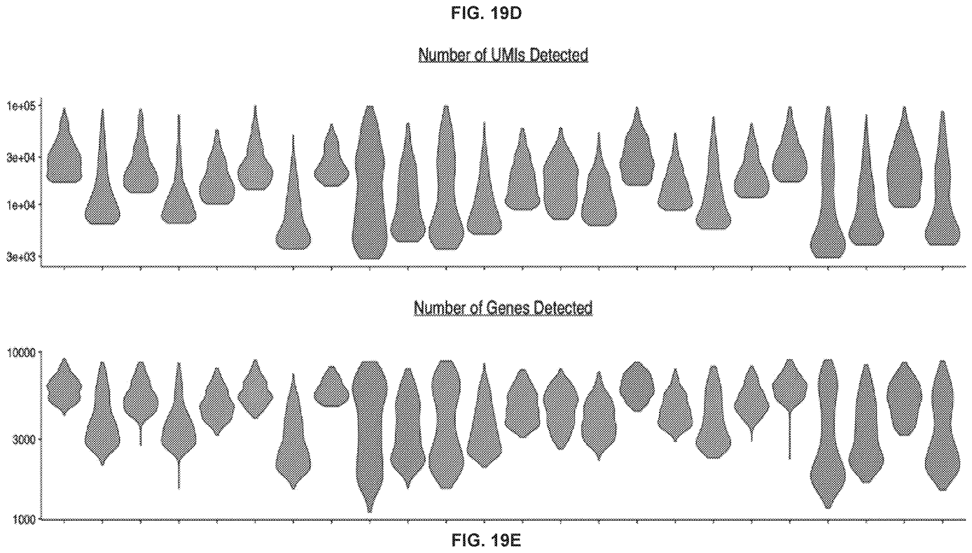

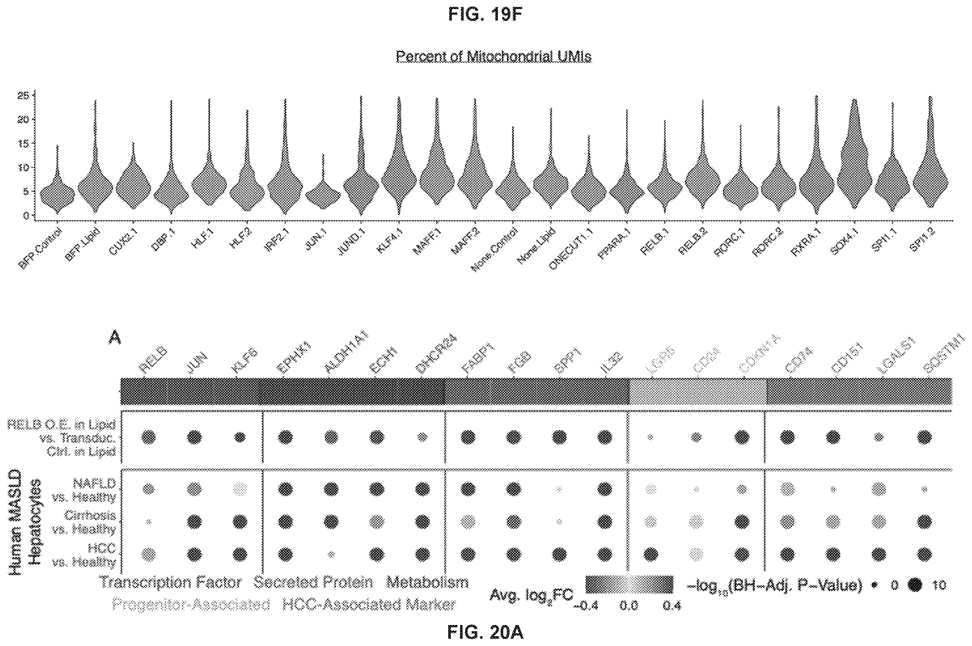

To validate MATCHA-nominated drivers of hepatocytes' metabolic adaptations, researchers conducted arrayed human in vitro genetic perturbations. HepG2 cell lines stably overexpressing TF isoforms were cultured in lipid-rich media, followed by: 1) scRNA-seq, to validate TF effects on hepatocytes' transcriptomic phenotypes (n=10,522 cells; median 417 cells per TF isoform), and 2) live-cell imaging and immunofluorescence, to validate TF effects on functional metabolic and cancer-associated phenotypes (FIGS. 6A, 19). Transcriptomic profiles of negative controls (non-transduced and BFP-transduced cells) clustered with each other by media condition but not transduction status, suggesting: 1) specificity of measured responses (via separation from TF-transduced cells), and 2) preserved responses to lipid-induced metabolic stress following transduction (FIG. 6B). As a positive control, it was confirmed that overexpression of PPARα in lipid-rich culture led to upregulation of known targets, including CPT1A, HMGCS2, PLIN2, G6PC1, and PCK1 (FIG. 19C). With successful recovery of known TF-target relationships, MATCHA-nominated TFs regulated hepatocytes' stress adaptations, focusing on RELB and SOX4 for their strength of effects on metabolic adaptation-associated transcriptional and functional states were evaluated.

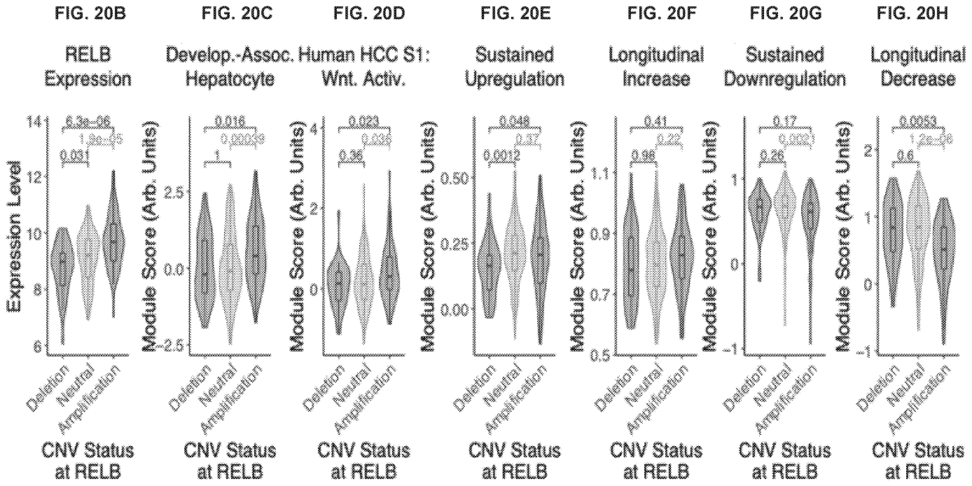

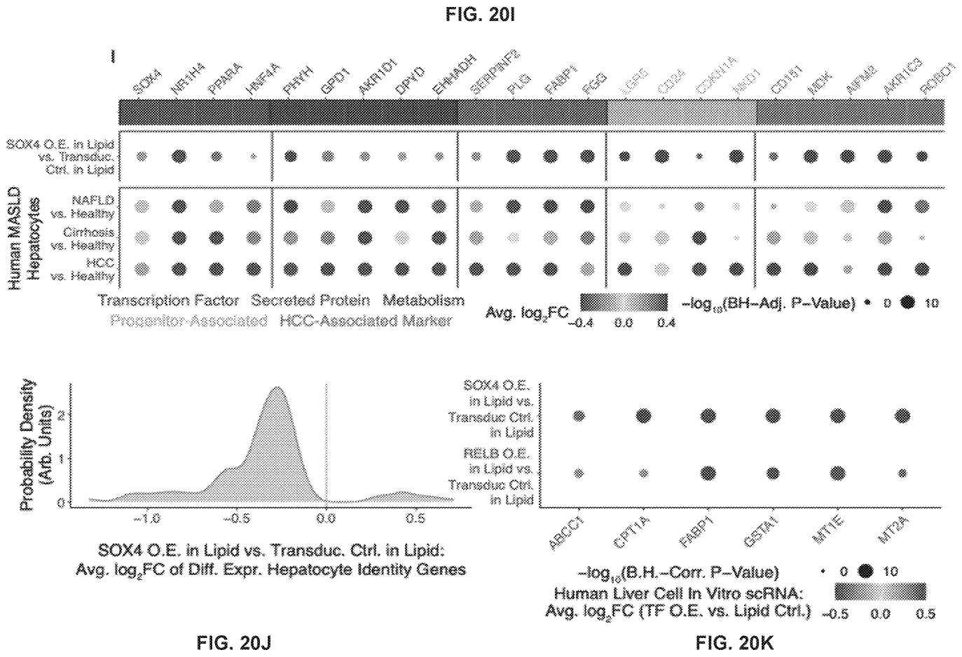

Overexpression of RELB (a member of the non-canonical NF-κB signaling complex) in lipid-rich media drove transcriptional shifts consistent with hepatocytes' in vivo long-term metabolic adaptations: elevation of the Longitudinal Increase, Sustained Upregulation, and HCC S1 signature programs, and declines of the Longitudinal Decrease and Sustained Downregulation programs (FIG. 6C). Specific RELB targets included increases in development-associated and regeneration-linked markers (e.g., CD24, CDKN1A, trend towards LGR5), decreases in hepatocyte secreted protein products (e.g., FGB, FABP1), and upregulation of HCC-associated genes (e.g., p62/SQSTM1, CD151, LGALS1, CD74) (FIG. 20A). Contextualizing RELB's effects against in vivo shifts, RELB drove effect sizes that were smaller than, but of comparable magnitude to, tumor-vs-healthy differences (FIG. 6C), suggesting both breadth and strength of RELB's effects on hepatocytes' stress adaptations. Towards in vivo MASLD relevance, RELB expression increased with fibrosis stage across multiple human cohorts, and RELB as a single marker stratified patient survival, associating with worsened outcomes (FIGS. 6D-6E).

As human in vivo evidence for RELB regulation of hepatocytes' stress adaptations, the question of how human HCC patients' copy number variations (CNVs) at the RELB locus altered expression of downstream gene programs was examined, analogous to a “natural genetic perturbation experiment” in human tumors. RELB CNVs drove significant changes in RELB transcription and predicted: significant increases in the development-associated, HCC S1, and Sustained Upregulation programs; significant decreases in the Sustained Downregulation and Longitudinal Decrease programs; and non-significant elevation of the Longitudinal Increase program (FIGS. 20B-20H). Thus, human in vitro genetic perturbations and human in vivo HCC CNVs support RELB as a driver of hepatocytes' stress adaptation programs, loss of cell identity, and development-associated and cancer-linked states.

Overexpression of SOX4 (SRY-box transcription factor 4, active during liver development) in lipid-rich media also depleted genes associated with hepatocyte cellular identity and function including TFs (e.g., NR1H4, PPARA, trend towards HNF4A), metabolic enzymes (e.g., GPD1, AKR1D1, DPYD, EHHADH), and secreted protein products (e.g., SERPINF2, PLG, FABP1, FGG) (FIGS. 6F, 20I). In contrast, SOX4 increased expression of developmental markers (e.g., CD24, WNT targets LGR5 and NKD1), as well as genes with functional effects in HCC (e.g., CD151, MDK, AIFM2, AKR1C2, ROBO1). As a broader, orthogonal examination of SOX4 driving loss of hepatocyte identity, the question of how SOX4 overexpression altered genes enriched in hepatocytes relative to all other cell types, as a proxy for hepatocytes' distinguishing features and cell identity was examined. 89% of statistically-significant hepatocyte identity-associated genes were downregulated by SOX4 expression, supporting SOX4 as driving loss of hepatocyte identity (FIG. 20J). Towards SOX4's human in vivo relevance, SOX4 expression increased with MASLD severity and stratified HCC patient survival, associating with worsened survival (FIGS. 6G-6H).

Towards RELB's and SOX4's functional consequences, overexpression of RELB and SOX4 decreased cells' lipid accumulation, but also caused accompanying dose-dependent increases in ROS accumulation (FIG. 61-6J, 6M). These effects may be mediated by shared downstream target genes linked to reduced lipid accumulation (e.g., increased ABCC1 and CPT1A, decreased FABP1) and elevated oxidative stress (e.g., decreased GSTA1, decreased MT1E and MT2A) (FIG. 20I). TF-mediated tradeoffs between lipid and ROS accumulation may align with prior work proposing a potentially protective role for lipid droplets, which can sequester lipid species that could otherwise drive lipotoxicity or increase ROS levels upon processing and oxidation. To connect these factors to longer-term disease outcomes, the effect of RELB and SOX4 on proliferation was further examined. Overexpression of RELB or SOX4 each increased nuclear accumulation of p53 protein (FIGS. 6K, 6M). SOX4 overexpression drove increased proliferation as measured by nuclear KI67; RELB overexpression did not drive significant changes in nuclear KI67, which could be interpreted as preserved proliferative capacity despite cellular stress (FIGS. 6L-6M; see Example 11 for discussion of p53 regulation of hepatocyte phenotypes and proliferation, especially connections to metabolism, development-associated states, WNT, and AP-1 signaling).

Through human in vitro genetic perturbation experiments and extensions to human cohorts, RELB and SOX4 were identified as causal regulators of stress-induced transcriptional and functional tradeoffs between hepatocyte identity and dysfunction-associated phenotypes, unifying hepatocytes' metabolic adaptations around specific regulatory nodes.

### Example 7: HMGCS2 is a Metabolic Mediator of Hepatocytes' Adaptation and Shifts Towards Cancer-Associated Phenotypes

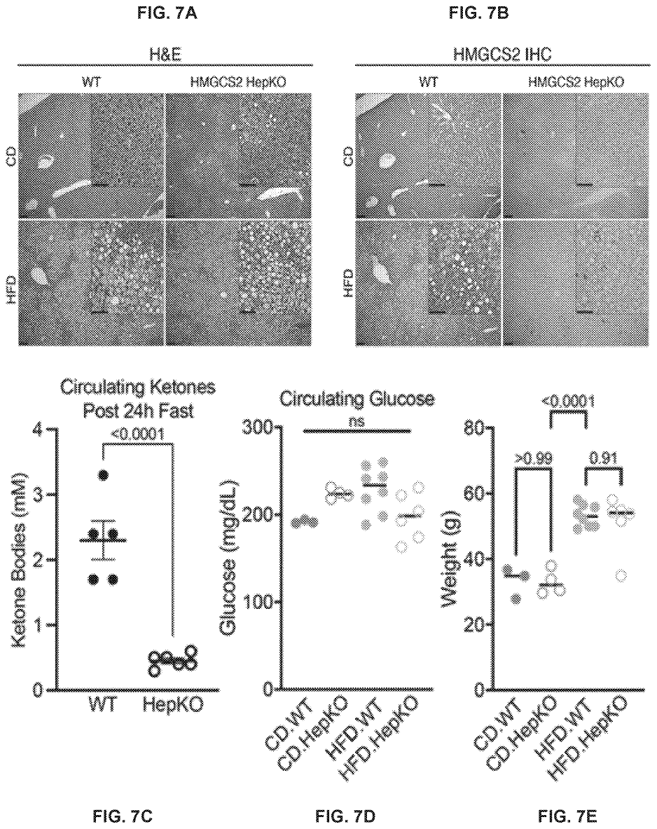

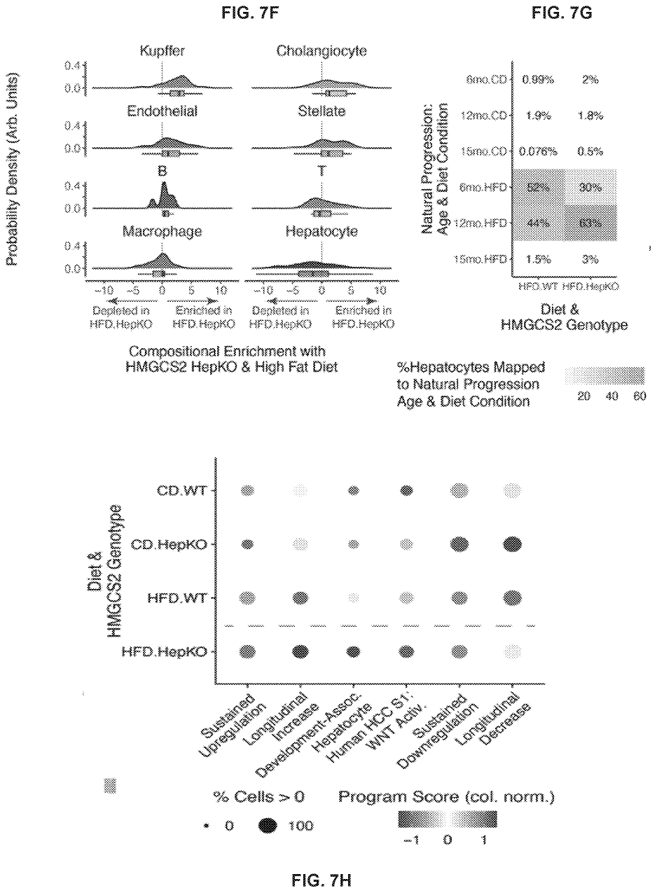

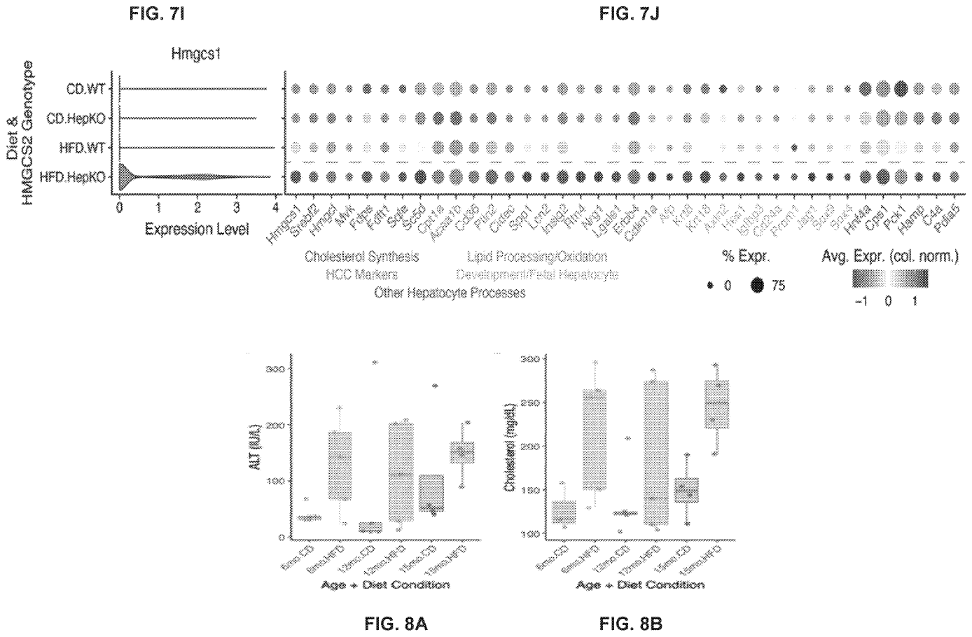

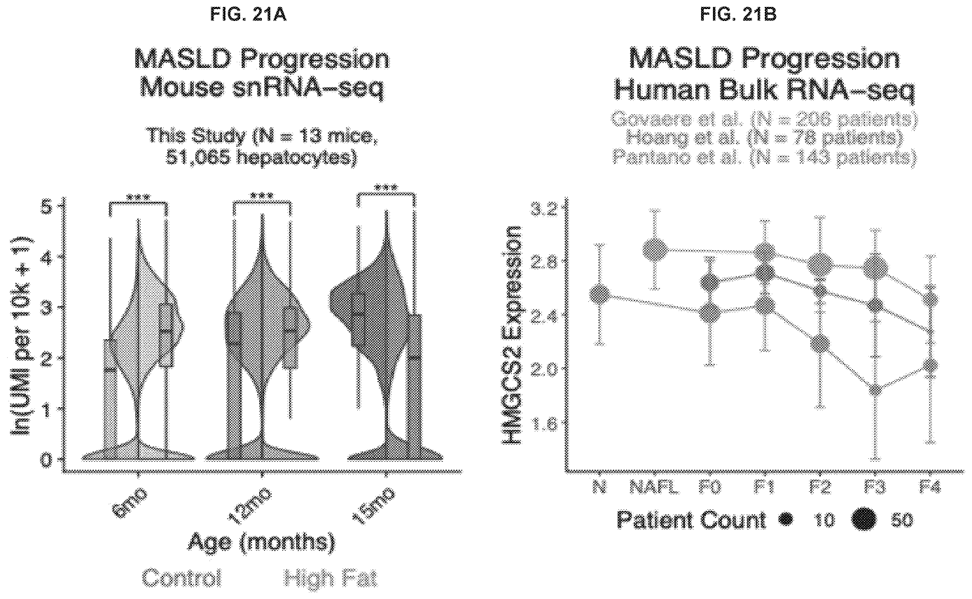

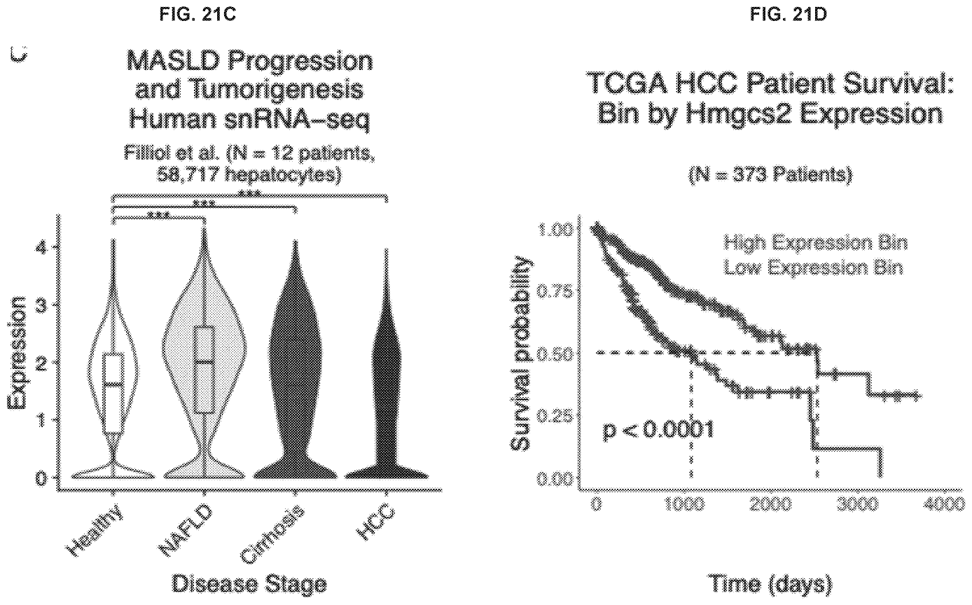

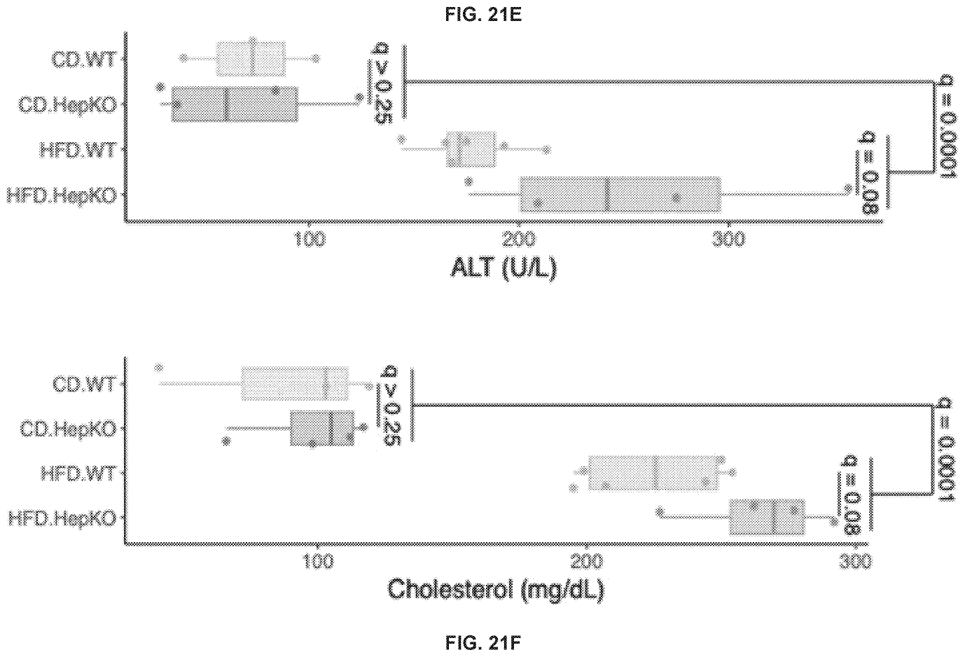

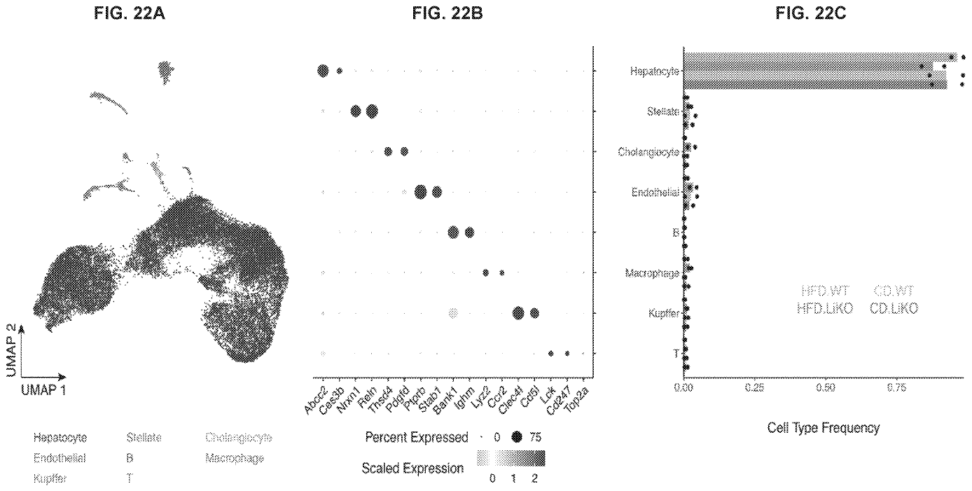

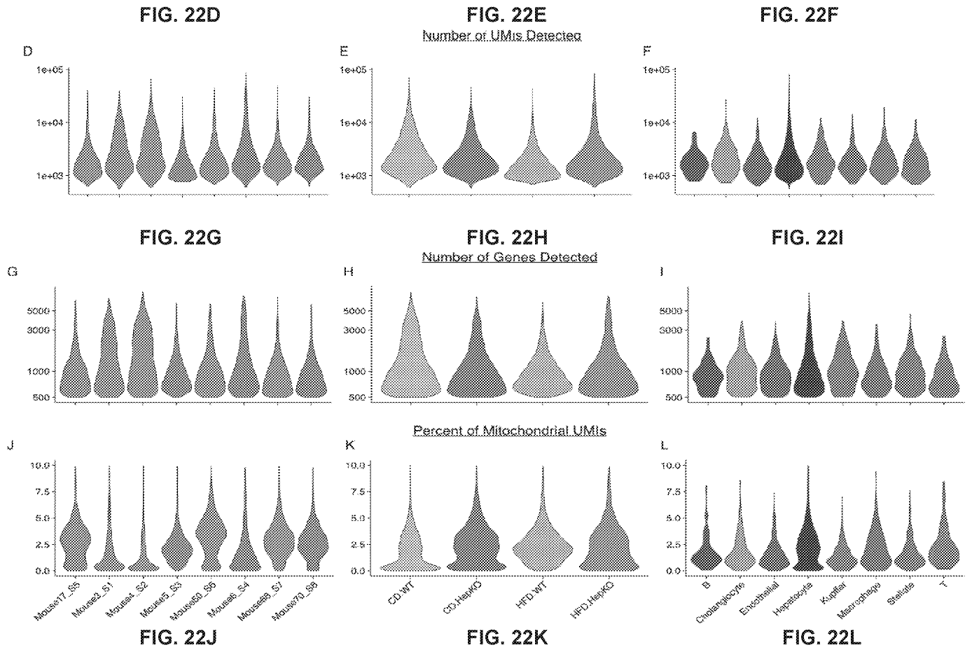

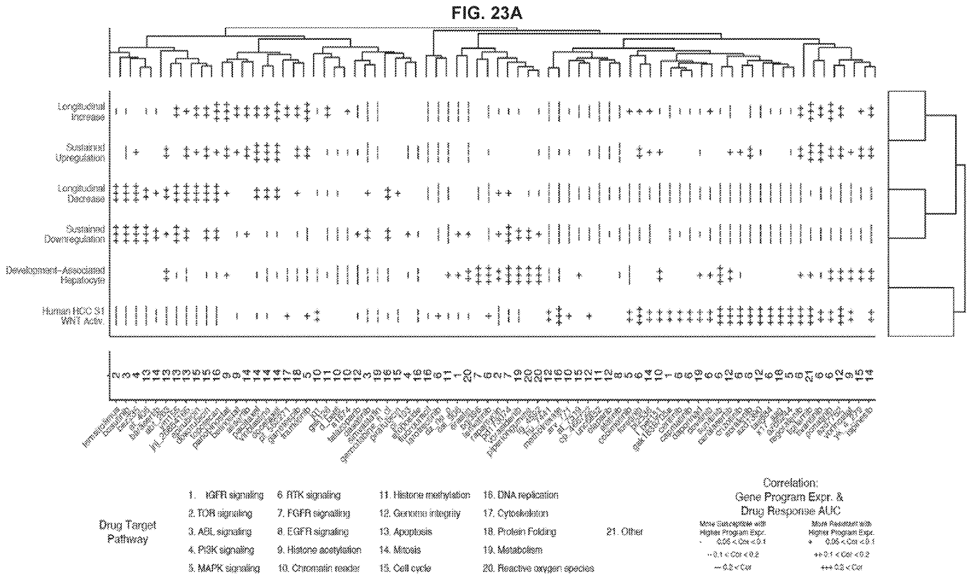

In an effort to demonstrate the in vivo importance of dynamic shifts in hepatocytes' stress adaptation programs: whether the programs merely represent correlative epiphenomena or instead drive disease-associated dysfunction. HMGCS2 (3-hydroxy-3-methylglutaryl-CoA synthase 2) and regulatory effects of ketogenesis-cholesterol metabolic rewiring, given HMGCS2's: 1) role as the rate-limiting enzyme of ketogenesis, 2) cross-species expression decreases with longitudinal metabolic stress, and 3) association with low expression and worsened HCC survival (FIGS. 21A-21D). A mouse model with hepatocyte-specific knockout of Hmgcs2 was generated, analogous to Hmgcs2 expression decreases with the natural stress adaptation progression (Hmgcs2^{flox/flox}; Alb-Cre) (FIGS. 21A-21D). After 6 months on either HFD or CD, steatosis occurred in mice on HFD with either wildtype (WT) or hepatocyte-specific HMGCS2 knockout (HepKO), but was comparatively less in CD mice of either genotype (FIG. 7A). Liver damage (measured by circulating transaminases) and cholesterol trended towards increases with the combination of HFD and HMGCS2 HepKO (FIGS. 21E-21F). Validating the HMGCS2 knockout, HMGCS2 abundance decreased throughout the liver lobule via immunohistochemistry and reduced circulating ketone bodies following a 24-hour fast (FIGS. 7B-7C). However, HMGCS2 HepKO did not alter circulating glucose concentrations or HFD-driven weight gains, indicating specificity of effects on ketogenesis (FIG. 7D-7E). To investigate the effects of HMCS2 loss on liver adaptations under chronic metabolic stress, snRNA-seq was conducted at the 6-month timepoint across HMGCS2 genotypes and diet conditions (N=8 mice, n=27,119 cells) (FIG. 22). Neighborhood-based analyses of the snRNA-seq data suggested compositional shifts specific to the metabolic stress response of HepKO mice, but not that of WT mice (FIG. 7F).

To understand the causal role of Hmgcs2 and ketogenesis on hepatocyte phenotypes, gene expression profiles of HFD.WT and HFD.HepKO hepatocytes were mapped to the natural progression of chronic metabolic stress (FIG. 7G). Approximately equal proportions of HFD.WT hepatocytes mapped to the natural progression 12-month (44%) and 6-month (52%) conditions. However, with HFD.HepKO, over twice as many hepatocytes mapped to the 12-month HFD condition (63%) as to the 6-month HFD condition (30%), indicative that HMGCS2 loss causes accelerated dysfunctional phenotypes associated with later stages of hepatocyte stress adaptation.

More directly, the question of how HMGCS2 HepKO under metabolic stress altered hepatocytes' longitudinal adaptation programs was examined. HMGCS2 HepKO on HFD led to extreme expression states, even relative to diet-induced shifts in WT mice: larger elevation of Sustained Upregulation, Longitudinal Increase, development-associated, and HCC S1 WNT Activation programs, but also further reductions of Sustained Downregulation and Longitudinal Decrease programs (FIG. 7H). To uncover specific targets, genes exhibiting emergent transcriptional shifts with metabolic stress and HMGCS2 HepKO were prioritized. Diverse cholesterol synthesis-related genes were upregulated with the combination of HMGCS2 HepKO and HFD (e.g., Hmgcs1, Srebf2, Hmgcl, Mvk), in line with compensation at the metabolic branch point between ketogenesis and cholesterol synthesis (FIGS. 7I-7J). Additionally, HMGCS2 HepKO on HFD induced cell states directly associated with long-term dysfunction, including HCC-linked intercellular signaling proteins (e.g., Spp1, Lcn2, Lgals1) and development-associated markers (e.g., Afp, Axin2, Hes1, Cd24a, Sox9). Loss of HMGCS2 on the background of HFD also accentuated decreases in genes associated with hepatocyte identity (e.g., Hnf4a) and function (e.g., Cps1, Pck1, Hamp, C4a, Pdia5). Finally, connecting HMGCS2 to MATCHA TF inference and experimental validation, HMGCS2 loss under chronic metabolic stress significantly upregulated Sox4.

Thus, in vivo genetic perturbation validated wide-ranging regulatory effects of HMGCS2 itself, but also more broadly the functional importance and interpretation of this work's stress adaptation programs. Premature HMGCS2 loss induced accelerated damage and extreme manifestations of transcriptional programs, including compensatory cholesterol synthesis, HCC phenotypes, development-associated states, and loss of hepatocytes' canonical functions. Experimental in vivo modeling of accelerated stress adaptation progression (through genetic manipulation) further supported adaptation programs derived in this work as fundamental, functionally-important axes of hepatocytes' responses to environmental stressors (FIGS. 24A-24N).

### Example 8: Contextualization of Diet-Only Chronic Stress Mouse Model Against Human Metabolic Stress Adaptation Progression

In an effort to connect phenotypes observed in the diet-only mouse model used in this work to those observed in human patients with the progression of MASLD and HCC, H&E and trichrome stains were blindly scored by a clinical liver pathologist, revealing that HFD mice typically demonstrate simple steatosis at 6 months and MASLD by 12 months (FIG. 8). HFD livers developed classic ballooning degeneration by 12 months, often associated with human patients' progression to more severe MASLD (FIGS. 1G-1H). Hepatocyte ballooning was confirmed by a second clinical liver pathologist and further validated by negative staining of CK8/18 (FIG. 1H). These temporal patterns of steatosis and fibrosis align with the well-known “burnt-out NASH” and “cryptogenic cirrhosis” phenotypes where hepatic fat is lost in human patients with end-stage liver disease.

As further contextualization of the scope of the model used in this work and resultant experimental datasets, a meta-analysis of 3,920 MASLD models found that ~75% of models have durations<6 months (mean=15 weeks), significantly shorter than the full MASLD progression. As part of this meta-analysis, the authors developed a 0-14 point scoring system for evaluating mouse models' metabolic stress phenotypes: 1 point each for obesity, insulin resistance, dyslipidemia, raised aminotransferases, lobular inflammation, portal inflammation, hepatocellular ballooning, NASH, fibrosis (presence/absence), maximum fibrosis stage (from 1-4), and hepatocellular carcinoma formation. Applying this scoring system to the study, the mouse model presented in this work scored a 12/14 (obesity, insulin resistance, dyslipidemia, raised aminotransferases, lobular inflammation, portal inflammation, hepatocellular ballooning, NASH, presence of fibrosis, maximum fibrosis stage 2, and hepatocellular carcinoma formation). In the authors' meta-analysis, only 3 (out of 3,920) models scored higher than a 12/14, indicative that the mouse model implemented in this work captures and mimics a variety of phenotypes important and associated with liver responses to metabolic overload (especially when evaluated in the context of the broader landscape of mouse modeling efforts). Other models' acceleration of progression through genotoxic chemicals or genetic manipulation increases differences from human disease pathogenesis and progression, especially when considered analyses targeted at understanding the natural dynamics of cellular adaptations to chronic stress. Thus, the diet-only nature of the model and longitudinal single-cell multi-omic readouts provide rich experimental and computational resources for probing cellular adaptations under stressful, diseased microenvironments of MASLD progression.

### Example 9: Connections Between Hepatocytes' Stress Adaptation Programs, Acute Regeneration Phenotypes, and Intercellular Signaling Drivers

Towards connections to the liver's intrinsic regenerative capacity, it was found that hepatocytes undergoing acute regeneration exhibit temporally-coordinated, internally-consistent expression patterns of the Longitudinal Increase and Decrease programs derived in this work, in line with hepatocytes' adaptations to metabolic stress drawing upon the liver's regenerative capabilities (FIG. 16J).

To more holistically examine intercellular signaling that may contribute to hepatocyte phenotypic alterations, the immune-biased live tissue scRNA-seq dataset was leveraged to infer ligands poised to regulate hepatocytes' metabolic adaptations (based on NicheNet; see Methods). Among the ligands predicted to regulate stress adaptation programs associated with increased disease severity and worsened tumor survival were neutrophil-derived MMP9, Kupffer cell-derived GDF3 and RTN4, endothelial cell-derived CTGF and EDNI, and T cell-derived LTB (FIG. 16F-16I). All of these ligands exhibited upregulated expression or increased sender cell abundances with high fat diet, complementing their literature associations with worsened MASLD severity, increased regeneration, and/or tumorigenesis contributions. Intercellular signaling interactions nominated through the analyses, but with comparatively less literature support, may represent promising directions for future experimental validation of cell-extrinsic mediators of hepatocyte dysfunction.

### Example 10: Contextualization of Computational Methods to Infer Transcription Factors Driving Arbitrary Gene Programs

While a wide variety of computational methods exist to estimate TF abundance or activity, the goal of identifying TFs that specifically regulate particular phenotypic gene programs remains an open problem. Below are highlighted generally-related approaches and distinctions from MATCHA's goal of mapping gene programs, enhancers, and program-specific TFs. The following descriptions are not intended to represent a comprehensive or all-encompassing list.
Examining differentially-expressed TFs (at the transcriptional level) or genome-wide TF motifs with differential accessibility (at the chromatin level) can prioritize TFs with generally altered activity, but not connect them to specific cellular phenotypes.
Genome-wide transcriptomic profiles can be translated into predicted TF activities through tools that leverage prior knowledge (e.g., response signatures, inferred interactomes, etc.). However, these tools provide a single TF score for each transcriptomic profile, but do not associate TFs with specific programs or distinguish their effects (or lack thereof) on specific cellular phenotypes.
Construction of gene regulatory networks can connect transcription factors to inferred downstream genes (with optional incorporation of multi-omic information). However, these tools most often focus on inference of TFs distinguishing distinct cell types (e.g., differentiation from one developmental stage to another), or simulated transitions across genome-wide transcriptomic profiles (e.g., represented as regions of density on a low-dimensional visualization such as UMAP or t-SNE). Thus, frameworks to connect TF regulons to user-specified gene programs of interest remain less clear. While gene regulatory network inference connects TFs to potential targets, the ability to define genes of interest and prioritize causal TFs has received less exploration. It remains difficult to identify TFs that target and modulate specific disease-associated phenotypes, rather than affecting the global cell state in a potentially less-focused or interpretable manner.

In contrast, MATCHA begins from a user-specified gene program and enables prioritization and nomination of TFs regulating that particular gene program, towards TFs driving specific aspects of cellular physiology. In this way, MATCHA enables discovery of tradeoffs or co-regulation across disease-linked axes of variation with distinct functions and repercussions (e.g., this work's Longitudinal Increase and Decrease programs, with opposing enriched processes, temporal trajectories, and disease connections). MATCHA: 1) leverages cell type- and/or tissue-specific transcriptomic and epigenetic relationships, towards capturing context-specific regulatory interactions, 2) can emphasize robust and generalizable gene program drivers by incorporating datasets spanning studies, cohorts and species, and 3) can uncover core, co-regulatory TFs capable of activating or repressing each of multiple gene programs simultaneously.

### Example 11: Contextualization of p53 Regulation of Hepatocyte Phenotypes

In the liver, p53 is known to have roles beyond its canonical anti-cancer genoprotective and antiproliferative functions. For instance, p53 modulates a variety of metabolic pathways (e.g., lipid processing, gluconeogenesis) in the liver, suggesting potential additional relevance for this TF in the context of metabolic overload. Towards metabolic functions of p53 during lipid overload, liver knockout of p53 in mice can produce steatosis, and p53 upregulation (via degradation of an upstream inhibitor) can alternatively attenuate fat accumulation. Additionally, elevated p53 activity has also been linked to progenitor-associated hepatocyte phenotypes. Multiple studies have shown that p53 can repress hepatocytes' lineage-determining transcription factor HNF4A, across reductions of HNF4A protein levels and promoter activity. p53 stabilization (e.g., via Mdm2 deletion) can induce progenitor markers in vivo; proposed mechanisms include p53 inducing inflammation that in turn promotes progenitor states or microRNA-mediated inhibition of HNF4A.

As literature support for KI67 accumulation and hepatocyte cycling despite elevated p53, both p53 and KI67 can increase with HCC grade. As specific examples, these genes were upregulated in tumors from a cohort of HBV-associated HCC patients relative to paired normal liver tissue; in a separate human HCC cohort, p53 levels correlated with the abundance of PCNA (another common cell cycle marker), and each associated with worse tumor grade. Importantly, stimulation of HepG2 cells with an intercellular signaling ligand drove the same directionality of changes in KI67 and p53 expression, demonstrating that these genes can exhibit correlated regulation under environmental perturbations. Additionally, in liver regeneration models using in vitro primary human hepatocytes and in vivo mouse livers, WNT signaling activation and AP-1 transcription factors can each suppress p53's anti-proliferative effects and abrogate p53-mediated cell cycle inhibition in hepatocytes; the transcriptional and epigenetic datasets support increased activities of each of these pathways over the course of MASLD progression. Thus, in particular cases in the liver, p53 and KI67 are not necessarily anti-correlated and can exhibit concordant regulation upon cellular perturbations.

During chronic stress, cells must balance individualistic survival against collective tissue-level functions. The question of how the liver manages longitudinal tradeoffs under environmental stressors was investigated through the paradigm of chronic metabolic overload, which precipitates progressive steatosis, inflammation, fibrosis, cirrhosis, and malignant transformation. In addition to its pressing clinical need, the biological context of NAFLD/NASH offers opportunities to understand how initial cellular adaptations connect to longer-term dysfunction and repercussions.

A diet-only mouse model that exhibits functional, histologic, and cellular phenotypes reminiscent of human NAFLD/NASH (e.g., CXCR6⁺CD8⁺ T cells, scar-associated macrophages, hepatocytes) was developed. Longitudinal, single-cell, multi-omic datasets across diet conditions (from early steatosis to late spontaneous tumorigenesis), along with harmonized human NAFLD/NASH/HCC transcriptomic and proteomic cohorts, provide rich computational resources on the intersection of aging and metabolic stress. Additionally, as the mouse model does not require genetic manipulation or exogenous chemical insults, it may serve as a broadly-translatable experimental resource for further investigations. HFD mice do not develop significant bridging fibrosis or nodule formation, whether due to lifespan differences between mice and humans, or opportunities for further model development for investigations focused on stellate cells and fibrosis.

Chronic metabolic stress induces development-linked and cancer-associated adaptations in hepatocytes, to the detriment of cellular identity and tissue functions. Hepatocytes increased genes related to 1) progenitor stages, 2) pro-survival and anti-apoptotic effectors, and 3) intercellular signaling (including WNT). In contrast, hepatocytes downregulated genes underpinning homeostatic roles, including diverse secreted proteins and metabolic enzymes. These findings were corroborated across multiple human cohorts, and recapitulated across epigenetic, transcriptomic, and proteomic readouts.

Suggesting discovery of generalizable stress adaptation responses, gene programs uncovered in this work exhibited extreme manifestations in acute regeneration and across HCC etiologies (consistent across etiologies including NAFLD/NASH, alcohol, and viral infections). These connections support further investigations on uncovered gene programs' generalizability as core, conserved axes of hepatocyte adaptation to diverse stressors. Such conserved axes in cross-disease cohorts could uncover broadly-applicable vs. disease-specific therapeutic vulnerabilities and patient stratification hierarchies. As potential mechanisms for conserved cross-disease stress adaptation programs, distinct molecular changes associated with each etiology may converge on similar intracellular mediators (e.g., diverse PAMPS or DAMPs converging on TLR activation). Alternatively, each etiology may drive recurrent microenvironmental or immune signals that in turn drive consistent hepatocyte phenotypes (e.g., regeneration contributions of both pro-inflammatory IL-6 and pro-fibrotic TGFB1).

Progressive decreases in lineage-determining HNF4A, decreases in genes mediating canonical hepatocyte functions, and increases in progenitor markers raise the question of how stress adaptations overlap or contrast with dedifferentiation. Partial hepatocyte dedifferentiation occurs following acute stressors like experimentally-induced partial hepatectomy and acetaminophen overdose, where subsets of hepatocytes downregulate canonical functions and assume fetal-like phenotypes to enable regeneration and restoration of liver mass. Genetically-induced priming of dedifferentiation improves hepatocyte survival during subsequent acute stressors, but long-term dedifferentiated states predispose to liver failure and worsened survival in HCC. In cancer, dedifferentiation is acknowledged as a recurrent hallmark, where cancer cells unlock fetal, plastic cell states associated with elevated proliferation and tumor progression. One potential model to unify these findings involves clinically-relevant chronic stresses driving partial-dedifferentiation-associated states even in non-transformed hepatocytes. In this model, the trajectory of hepatocytes' progressive stress adaptations would increasingly draw upon the liver's regenerative capacity for improved survival of individual cells, but with deleterious repercussions: 1) maladaptive tradeoffs with the liver's tissue-level functions and homeostatic setpoints, and 2) early induction and priming of similar transcriptional programs as occur with tumorigenesis and worsened cancer outcomes. Towards testing this model, future demonstrations functionally linking stress adaptation and de-differentiation (especially early in disease progression in non-transformed hepatocytes) may further delineate transcriptional and epigenetic contributions to liver failure and elevated cancer incidence.

Extending hepatocytes' immediate adaptations, it was also found that WNT signaling members and HCC markers exhibited increased chromatin accessibility months prior to transcriptional upregulation with tumorigenesis. These findings suggest stress adaptations incurring not just direct changes, but also epigenetic priming for later activation of cancer-associated states. In other organs and diseases, AP-1 synergizes with context-specific TFs to maintain poised epigenetic landscapes and memory of past inflammation; future work could investigate the minimal requirements and timescales needed for epigenetic dysregulation and eventual phenotypic manifestations. For instance, in the skin and pancreas, inflammatory bouts lasting only a handful of days are sufficient to drive effects upon triggers administered months later (e.g., improved wound healing, increased cancer risk), which would suggest even short stressors can drive persistent tissue memory and altered tissue function. In contrast, in the nasal epithelium, modulation of cytokine signaling can partially revert dysfunctional progenitor memory of allergic memory, towards remnant plasticity and therapeutic capability to restore (aspects of) homeostatic baseline. Hepatocytes' primed accessibility at AP-1 binding motifs and WNT-related loci occurred at relatively early disease stages which precede when many patients exhibit symptoms. Thus, future investigations into the timescales of stress adaptations' initiation and persistence are motivated by not only fundamental biological understanding but also longer-term clinical relevance, of whether hepatocyte tissue memory and lasting maladaptive phenotypes may be seeded prior to disease diagnosis. With weight loss capable of stabilizing or even reversing histologic features of NAFLD/NASH if sustained, whether hepatocytes' memory of chronic stress is reversible or establishes lasting (mal) adaptive responses even after return to normal weights could shape long-term implications of ongoing trials and novel therapeutic avenues for NAFLD/NASH.

To extend from discovering axes of cellular stress adaptations to uncovering their causal regulators, MATCHA, a computational framework to prioritize TFs driving arbitrary, user-specified gene programs, was developed. MATCHA infers gene program—co-accessible enhancer—causal TF triads by leveraging: 1) multimodal-omics data across layers of genome regulation, temporal trajectories, and species, and 2) context-dependent regulatory relationships ensuring output predictions capture disease- and cell type-specific TF activity. When applied to externally-defined biological processes, MATCHA recovered their well-established ground truth drivers. When applied to uncover “central hub” TFs with strong co-regulatory connections across multiple stress adaptation programs derived in this work, MATCHA re-discovered targets of multiple Phase III clinical trials, but also highlighted comparatively less-explored TFs that were experimentally validated. A strength of MATCHA is its emphasis on incorporating tissue and cell type-specific data as the basis for computational inference of regulatory relationships, capturing context dependence of chromatin landscapes and TF activity. Future improvements of MATCHA could include prioritizing sets of TFs needed to drive the breadth of a gene program (e.g., beyond top TFs prioritized in isolation that may have overlapping, noncomprehensive target genes). Alternatively, in addition to predicting TF regulatory effects on a gene program, it is possible to elucidate differential gene regulatory network structures, which could reveal alterations in enhancer binding and altered downstream target genes even within a given TF.

Validating MATCHA predictions, RELB and SOX4 indeed regulated hepatocytes' stress adaptation transcriptional programs and functional metabolic phenotypes in human in vitro genetic perturbation experiments. RELB constitutes the transcriptional effector of the non-canonical NF-κB signaling pathway. In the liver, significant prior work investigated cytoplasmic regulation of NF-κB kinase complexes, as well as canonical NF-κB signaling's roles in tumorigenesis and hepatocyte survival under inflammatory conditions. However, canonical and non-canonical NF-κB differ significantly in terms of input signaling mechanisms and output phenotypic effects: they are activated by distinct ligands, involve separate intracellular interactions, and culminate in different TFs. Thus, the role of RELB in hepatocytes' progressive remodeling under metabolic stress has received less. This work points towards novel contributions of RELB to driving hepatocytes' (mal) adaptations to chronic stress, across disease associations, transcriptional targets, and functional effects. Likewise, SOX4 is active during fetal liver development and epithelial progenitor fate specification towards cholangiocytes. In HCC, SOX4 predicts worsened survival, and ectopic Sox4 overexpression in vivo decreases hepatocyte identity features and drives metaplasia. However, SOX4's involvement in adult hepatocytes' metabolic regulation and adaptations has been less explored. This work supports SOX4's involvement in mature hepatocytes' stress responses, with especially strong links to downregulation of canonical hepatocyte functions that align with the posited de-differentiation interpretation of hepatocytes' stress adaptations. These results additionally support that even individual regulatory nodes can be sufficient to co-regulate and couple phenotypes with opposing functional associations and temporal trajectories during progressive stress adaptations.

While this human in vitro model allowed for the validation of TFs' cell-intrinsic effects, future work could define their upstream activating signals (e.g., through co-culture or organoid systems enabling dissection of intercellular interactions, biochemical cues, mechanical microenvironments, etc.). These intercellular signaling analyses nominated ligands associated with both NAFLD/NASH severity and activation of RELB or SOX4 (Example 9): LTB activates both canonical and non-canonical NF-κB signaling, supports successful acute regeneration and mouse survival after partial hepatectomy, and was predicted to drive hepatocytes' Early Upregulation program. Likewise, TGFB1 activates SOX4 and was predicted to drive hepatocytes' Longitudinal Increase and progenitor-associated programs. SOX4 can additionally be activated by WNT signaling, whose activation was supported at epigenetic, transcriptomic, and proteomic levels.

Towards demonstrating beneficial-vs-maladaptative repercussions of dynamic shifts in stress adaptation programs, these analyses highlighted opposing temporal trajectories of ketogenesis and cholesterol synthesis. These pathways compete for the same starting precursor metabolite of acetoacetyl-CoA (product of fatty acid oxidation). Recent work demonstrated that acetoacetyl-CoA accumulation may increase liver tumorigenesis risk via histone acetylation modifications that facilitate accessible, permissive chromatin landscapes. However, as hepatocytes allocate flux through these metabolic pathways for processing acetoacetyl-CoA, their downstream products have opposing associations with hepatocyte health: free cholesterol can directly cause lipotoxicity, while hepatocytes lack the necessary enzyme to catabolize ketone bodies and therefore export them to other organs. Demonstrating maladaptive effects of ketogenesis decreases during naturally-occurring stress adaptations, metabolically-stressed Hmgcs2^{-/-} hepatocytes exhibited accelerated shifts towards phenotypes characteristic of later stages of chronic stress exposure: 1) compensatory increases in cholesterol synthesis enzymes, 2) extreme manifestations of adaptation gene programs; 3) upregulation of HCC markers and progenitor-associated genes including Sox4; and, 4) downregulation of lineage-determining Hnf4a and canonical hepatocyte functions. It was previously shown that ketone bodies regulate intestinal stem cell regenerative capacity via epigenetic interactions; future work could further elucidate precise molecular mechanisms and interactions by which ketogenesis, cholesterol synthesis, and associated metabolites regulate the hepatocyte response to metabolic stress.

One final important question is whether stress adaptation programs uncovered in this work are necessarily co-regulated or can be disentangled for selective modulation. Pareto analyses have investigated core archetypes of cellular contributions to collective functions, with hepatocytes exhibiting spatially-structured tradeoffs and division of labor between tissue-scale tasks. However, these analyses focused on steady-state healthy hepatocytes and did not consider dynamic axes of disease progression. Thus, disease contexts present additional complexity (but also opportunities) in understanding how cells and tissues navigate the state space of potential phenotypes and maintain essential functions despite external stressors. This work sought to demonstrate the existence and functional implications of chronic stress adaptation programs by focusing on validation of transcriptional (RELB, SOX4) and metabolic (HMGCS2) mediators of wide-ranging dysfunction. However, future work could attempt to decouple these programs, therapeutically activating only beneficial features while mitigating otherwise-linked deleterious phenotypes. For instance, engineered transcriptional activators may enable support of both cellular survival and tissue function without priming of phenotypes associated with worsened cancer outcomes. Alternatively, targeting key enzymes to tune relative metabolic fluxes would provide greater control of tissue homeostatic setpoints, towards buffering of healthy function against environmental stressors.

In this work, it was demonstrated how long-term stress drives adaptations that balance immediate cellular survival, homeostatic tissue functions, and long-term dysfunction. Through dissection of hepatocytes' temporal adaptation trajectories, computational methods development to nominate cell-extrinsic and cell-intrinsic drivers, and experimental validation via human in vitro and mouse in vivo genetic perturbations, diverse axes of hepatocyte adaptation were coalesced around their specific causal factors. Ultimately, this work provides a foundation for revealing the principles behind cellular and tissue decision-making during stress, translating complex descriptions of disease dysfunction into unifying core causes, and deriving fundamental connections of how even early stress can precipitate cellular adaptations and tradeoffs which lead to long-term dysfunction.

The following materials and methods were used in the above examples.

### Mouse Husbandry and High Fat Diet

Natural progression cohort
Hmgcs2 KO cohort

### Mouse Functional and Histological Characterization

Body weight
Albumin
H&E
Sirius Red
Oil Red O
Cholesterol
ALT
Glucose tolerance
IHC (CK8/18, Hmgcs2)
Tumor MRI
Normal tissue+tumor histology
   Frozen-Tissue Nuclei Isolation for Tandem snRNA-Seq and snATAC-Seq

For each sample, the following solutions and volumes were prepared for isolation of nuclei for tandem Seq-Well snRNA-seq and 10× v1.1 snATAC-seq. All solutions were pre-chilled and kept on ice except when actively handling the tissue or nuclei solution, and then immediately returned to ice.
Base buffer: 100 μL of 1M Tris-HCl pH 7.4, 20 μL of 5M NaCl, 30 μL of 1M MgCl₂, 1000 μL of 10% bovine serum albumin.
Wash buffer+RNase inhibitor: 230 μL of base buffer, 20 μL of 10% Tween-20, 1.7 mL of nuclease-free water, 50 μL of Sigma-Aldrich Protector.
Wash buffer+digitonin: 230 μL of base buffer, 20 μL of 10% Tween-20, 1.746 mL of nuclease-free water, 4 μL of 5% digitonin.
1× lysis buffer: 230 μL of base buffer, 20 μL of 10% Tween-20, 20 μL of 10% Nonidet P40 substitute, 1.736 mL nuclear-free water.
Lysis dilution buffer: 230 μL of base buffer, 1.77 mL of nuclease-free water.
0.1× lysis buffer: 200 μL of 1× lysis buffer, 1.75 mL of lysis dilution buffer, 50 μL of Sigma-Aldrich Protector.
Diluted nuclei buffer: 48.75 μL of 20× Nuclei Buffer (from 10× v1.1 scATAC-seq reagent kit), 926.25 μL of nuclease-free water, 25 μL of Sigma-Aldrich Protector.
PBS+1% BSA+RNase inhibitor: 875 μL PBS, 100 μL of 10% BSA, 25 μL of Sigma-Aldrich Protector.

Flash-frozen pieces of liver tissue were kept on dry ice; if needed, a smaller piece of tissue (~2-3 mm diameter) was cut using a scalpel on a petri dish on dry ice. The tissue piece was placed into a Miltenyi C tube containing 2 mL of 0.1× lysis buffer, then homogenized using 2 iterations of the m_spleen_01 program on the gentleMACS tissue dissociator. Half of the solution was passed through a 40 μm filter pre-wet with 1 mL wash buffer+RNase inhibitor, and the other half of the solution was passed through a separate 40 μm filter prewet with 1 mL wash buffer+digitonin. The C tube was washed with 1 mL of wash buffer+RNase inhibitor to capture remnant nuclei stuck to the tube side or lid, and 500 μL was transferred to each separate tube. Each solution was transferred to a separate 15 mL Falcon tube and centrifuged for 10 min at 500 g and 4° C., with brake set to 5 (out of a maximum of 10). The supernatant was aspirated, and each pellet was resuspended in 100 μL of diluted nuclei buffer. Two 35 μM filters were pre-wet with diluted nuclei buffer, and flow-through from the pre-wetting solution was removed to leave the tubes empty; each nuclei solution was then passed through the separate pre-wet 35 μM filters using a P200 pipette. Each set of nuclei was counted using a hemocytometer, followed by snRNA-seq and snATAC-seq as previously described:
15,000 nuclei in 200 μL of PBS+1% BSA+RNase inhibitor as input to Seq-Well S3, as described in Hughes*, Wadsworth II*, Gierahn*, et al., Immunity (2020), the disclosure of which is incorporated herein by reference in its entirety for all purposes.
7,000*1.53 nuclei in 5 μL of diluted nuclei buffer as input to 10× snATAC-seq, as described in Chromium Next GEM Single Cell ATAC Reagent Kits v1.1 User Guide, CG000209 Rev F

A similar protocol was followed for nuclei isolation from: 1) the frozen tumor and adjacent normal tissue cohort, and 2) the Hmgcs2 HepKO cohort. The following modifications were made:
As snATAC-seq was not conducted, the splitting of nuclei into a tube containing wash buffer+digitonin and subsequent parallel processing steps were omitted; all nuclei were passed through a single 40 μm filter pre-wet with 1 mL wash buffer+RNase inhibitor.
At the 35 μm filter step, instead of diluted nuclei buffer, the filter was pre-wet with PBS+1% BSA+RNase inhibitor solution.

Samples were sequenced on an Illumina NextSeq 500/500, NextSeq 2000, or NovaSeq 6000.

### Lentiviral Production

Plasmids encoding TF ORFs were obtained as generated in Joung et al., Cell (2023), the disclosure of which is incorporated herein by reference in its entirety for all purposes; deposited in Addgene MORF Collection.

600,000 Lenti-X 293T cells were plated in 2 mL of media (DMEM+10% FBS+1% penicillin-streptomycin) in a 6-well plate and incubated overnight at 37° C. and 5% CO₂ (Day 1). The following afternoon (Day 2), 1 μg of psPAX2, 0.33 μg of pMD2.G, 1.33 μg of ORF plasmid, 5.3 μL of P3000 reagent, and 125 μL of Opti-MEM were mixed, added to a solution of 6.7 μL of L3000 and 125 μL of Opti-MEM (not mixed), and incubated for 15 min at room temperature. The mixture was added to the Lenti-X 293T cells and incubated overnight. The following morning (Day 3), Lenti-X 293T media was replaced. The following afternoon (Day 4), Lenti-X 293T media was harvested and replaced; the overnight media was passed through a 0.45 μm low protein binding filter, combined with Lenti-X Concentrator at a 3:1 media:Lenti-X ratio, and stored overnight at 4° C. The following afternoon (Day 5), Lenti-X 293T media was again harvested and added to the previous day's media and Lenti-X Concentrator solution at 4° C. for 30 min. The media was spun at 1500 g for 45 min at 4° C. Supernatant was aspirated, and the pellet was resuspended in 160 μL for storage at −80° C. in 20 μL aliquots.

### Lentiviral Transduction

For each TF ORF, 500,000 HepG2 cells were plated in 2 mL of media (Advanced DMEM/F12+10% FBS+1% penicillin-streptomycin) in a 6-well plate and incubated overnight at 37° C. and 5% CO₂ (Day 1). The following morning (Day 2), lentiviral stocks of each TF ORF and polybrene were thawed to room temperature. Cells' media was replaced with a 10 μg/mL solution of polybrene in 2 mL of HepG2 media, followed by addition of 8 μL of lentivirus to separate HepG2 wells (i.e., arrayed format; 1 TF ORF per well). Cells were incubated with lentivirus overnight, and media was replaced the following morning (Day 3). The following afternoon (Day 4), media was replaced with a 1 μg/mL solution of puromycin in HepG2 media.

To produce stable, arrayed HepG2 lines overexpressing each TF, cells were maintained in puromycin-containing media to select for successful transduction, with puromycin-containing media being changed every 2-3 days. Upon expanding to reach confluency in a 6-well plate, each TF ORF-overexpressing HepG2 line was passaged and replated in a 10 cm dish. Upon reaching confluency in a 10 cm dish, each TF ORF-overexpressing HepG2 line was passaged to freeze down cell stocks (1,000,000 cells in 1 mL of 90% FBS+10% DMSO at −80° C. in a Mr. Frosty Freezing Container), followed by subsequent functional and transcriptomic assays in the metabolic stress of lipid-rich media.

Liver Cell Lipid Culture, scRNA-Seq, Functional Imaging, and Immunofluorescence

HepG2 cells overexpressing each TF ORF were seeded in a 96-well plate at a density of 4,000 cells/well. As lipid-rich, metabolically-stressful media, TF ORF-overexpressing cells were cultured in Advanced DMEM/F12 media containing 10% FBS, 500 μM palmitic acid, 100 μM oleic acid, and 1% penicillin-streptomycin. As controls, BFP-transduced HepG2 cells and non-transduced HepG2 cells were each cultured in lipid-rich media or control media (Advanced DMEM/F12 media containing 10% FBS, 600 mM BSA control, and 1% penicillin-streptomycin). Each well received 200 μL of its respective media condition (i.e., TF ORF-overexpressing HepG2's in lipid-rich puromycin-containing media; BFP-transduced HepG2's in lipid-rich or control puromycin-containing media; non-transduced HepG2's in lipid-rich or control puromycin-free media). Media was changed 2 and 4 days after seeding, with assays occurring 7 days after seeding.

For scRNA-seq, HepG2 cells for each condition were seeded across triplicate wells. 7 days after seeding, cells were passaged, and triplicate wells for each condition were pooled and used as input for scRNA-seq using Seq-Well S.

For functional imaging, cells were stained with BODIPY (2 μM final concentration), CellROX Deep Red (1:500 final dilution), and Hoechst 33342 (5 μg/mL final concentration) in PBS at 37° C. and 5% CO₂. After 30 min incubation, cells were washed 3 times with PBS, followed by imaging on an Opera Phenix at 37° C. and 5% CO₂.

After functional imaging, cells were fixed in a solution of 4% paraformaldehyde in PBS for 10 minutes, followed by a 3× wash with PBS. Cells were then permeabilized with 0.1% Tween (KI67 imaging wells) or 0.3% Triton-X (total p53 imaging wells) in PBS with 1% BSA for 10 min, followed by overnight incubation at room temperature with the primary antibody (ThermoFisher SolA15 for KI67, Cell Signaling Technology 7F5 for total p53). The following morning, cells were incubated with secondary antibody and imaged on an Opera Phenix.

scRNA-Seq and snRNA-Seq OC, Filtering, and Annotation

Bcl2fastq was used to convert sequencing reads into bcl files for alignment with either the DropSeq pipeline (live-tissue scRNA-seq samples) or STARsolo (frozen-tissue snRNA-seq and HepG2 scRNA-seq samples). Mouse samples were aligned to the mm10 reference genome, and human samples were aligned to the Hg38 reference genome with the BFP sequence appended (to validate successful transduction via BFP positive control samples).

Cells were first filtered based on number of detected genes, detected UMIs, and percent of mitochondrial counts (FIGS. 8-9, 13, 19, 21). The number of principal components was chosen based on an automated elbow-based selection criterion, followed by construction of nearest-neighbor and shared nearest-neighbor graphs and UMAP visualization. Clustering was implemented using the Leiden algorithm, and resolution was chosen based on a parameter scan and maximization of silhouette coefficient. Clusters solely distinguished by quality-associated or doublet-associated features were removed: 1) high expression of mitochondrial genes or well-established markers of low-quality cells (e.g., MALAT1, NEAT), or 2) co-expression of markers for mutually-exclusive lineages (e.g., immune and epithelial cells). Cells were annotated based on canonical markers established in prior liver atlases, and clusters corresponding to broad lineages were merged for further subclustering (i.e., epithelial, immune, structural/stromal). Within each lineage, variable gene selection, PCA, nearest-neighbor graph construction, clustering, and UMAP visualization were re-performed; clusters distinguished by markers of other lineages were removed as doublets. Lineage-specific subclustering and doublet removal was repeated 2-3 times within each lineage to ensure robust heterotypic doublet identification.

snATAC-Seq OC, Filtering, and Annotation

CellRanger was used for processing and alignment to mm10-2020-A_arc_v2.0.0. Cells were filtered based on number of detected peaks, percent of reads in peaks, nucleosome signal, and TSS enrichment. The number of latent semantic indexing components was chosen based on an automated elbow-based selection, followed by construction of nearest-neighbor and shared nearest-neighbor graphs and UMAP visualization. Iterative subclustering and doublet removal were implemented as described in “scRNA-seq and snRNA-seq QC, Filtering, and Annotation”, using chromatin-based gene activity scores instead of canonical marker genes.

### Metabolic Adaptation Gene Program Derivation and Driver Gene Identification

At each timepoint for each of the scRNA-seq and snRNA-seq hepatocyte datasets, the average log 2 (fold-change) was calculated across all genes detected in at least 25% of cells (chosen based on differential expression benchmarking analyses examining robust parameter estimates and statistical outputs as a function of average expression and detection rate180). To promote prioritization of generalizable gene programs across species, researchers additionally incorporated differential expression information from Govaere et al.'s human bulk RNA-seq cohort, using limma-trend to model gene expression as a function of MASLD stage. See below for qualitative descriptions of the temporal patterns and trajectories captured by each gene program (defined quantitatively further below):
Sustained Upregulation program: Genes whose elevation is maintained over time.
Sustained Downregulation program: Genes whose lessening is maintained over time.
Longitudinal Increase program: Genes progressively elevated with long-term chronic stress exposure.
Longitudinal Decrease program: Genes progressively lessened with long-term chronic stress exposure.
   Different genes are preferentially retained and measured in live-tissue scRNA-seq (nuclear+cytoplasmic mRNA) vs. frozen-tissue scRNA-seq (nuclear mRNA only). As a result, a gene may have a large fold-change in one dataset, but non-detection or sparse detection in the other dataset (in turn causing fold-changes that are inestimable or equal to 0). To maximize insights from both the live-tissue scRNA-seq dataset (higher molecular capture given the retention of cytoplasmic mRNA) and frozen-tissue snRNA-seq dataset (higher hepatocyte abundance), researchers sought to leverage complementary information from each dataset during gene program derivation. Researchers followed the principle that genes should follow a given temporal trajectory (e.g., Sustained Upregulation) in at least one dataset, while allowing for non-detection/sparse detection in the other dataset (e.g., positive fold-changes in one dataset and non-negative fold-changes in the other dataset). As a result, gene programs were derived by filtering to retain genes matching the general structure shown below:
(Condition_{Live_scRNA} OR Condition_{Frozen_snRNA}) AND Condition_{Govaere}

See below for each gene program's definitions of Condition_{Live_scRNA}, Condition_{Frozen_snRNA}, and Condition_{Govaere}, where log 2FC represents average log 2 (fold-change) at the noted timepoint between hepatocytes from high fat vs. control diet mice in live-tissue scRNA-seq or frozen-tissue snRNA-seq datasets, and β_{Govaere_limmatrend} represents the regression coefficient of gene expression as a function of disease stage in Govaere et al.:
Sustained Upregulation:
   Condition_{Live_scRNA}: Across all timepoints, consistent upregulation in live-tissue scRNA-seq and non-negative fold-changes in frozen-tissue snRNA-seq

$${log}_{2} {FC}_{Live \_ 6 mo} > 0 AND {log}_{2} {FC}_{Live \_ 12 mo} > 0 AND {log}_{2} {FC}_{Live \_ 15 mo} > 0 AND {log}_{2} {FC}_{Frozen \_ 6 mo} \geq 0 AND {log}_{2} {FC}_{Frozen \_ 12 mo} \geq 0 AND {log}_{2} {FC}_{Frozen \_ 15 mo} \geq 0$$

Condition_{Frozen_snRNA}: Across all timepoints, consistent upregulation in frozen-tissue snRNA-seq and non-negative fold-changes in live-tissue scRNA-seq

$${log}_{2} {FC}_{Frozen \_ 6 mo} > 0 AND {log}_{2} {FC}_{Frozen \_ 12 mo} > 0 AND {log}_{2} {FC}_{Frozen \_ 15 mo} > 0 AND {log}_{2} {FC}_{Live \_ 6 mo} \geq 0 AND {log}_{2} {FC}_{Live \_ 12 mo} \geq 0 AND {log}_{2} {FC}_{Live \_ 15 mo} \geq 0$$

Condition_{Govaere}: Increased expression with disease stage

$${β}_{Govaere \_ limmatrend} > 0$$

Sustained Downregulation program:
   Condition_{Live_scRNA}: Across all timepoints, consistent downregulation in live-tissue scRNA-seq and non-positive fold-changes in frozen-tissue snRNA-seq

$${log}_{2} {FC}_{Live \_ 6 mo} < 0 AND {log}_{2} {FC}_{Live \_ 12 mo} < 0 AND {log}_{2} {FC}_{Live \_ 15 mo} < 0 AND {log}_{2} {FC}_{Frozen \_ 6 mo} < 0 AND {log}_{2} {FC}_{Frozen \_ 12 mo} \leq 0 AND {log}_{2} {FC}_{Frozen \_ 15 mo} \leq 0$$

Condition_{Frozen_snRNA}: Across all timepoints, consistent downregulation in frozen-tissue snRNA-seq and non-positive fold-changes in live-tissue scRNA-seq

$${log}_{2} {FC}_{Frozen \_ 6 mo} < 0 AND {log}_{2} {FC}_{Frozen \_ 12 mo} < 0 AND {log}_{2} {FC}_{Frozen \_ 15 mo} < 0 AND {log}_{2} {FC}_{Live \_ 6 mo} \leq 0 AND {log}_{2} {FC}_{Live \_ 12 mo} \leq 0 AND {log}_{2} {FC}_{Live \_ 15 mo} \leq 0$$

Condition_{Govaere}: Decreased expression with disease stage

$${β}_{Govaere \_ limmatrend} < 0$$

Longitudinal Increase program:
   Condition_{Live_scRNA}: Progressive increases from 6-month to 15-month timepoints in live-tissue scRNA-seq and non-decreases in frozen-tissue snRNA-seq

$${log}_{2} {FC}_{Live \_ 15 mo} > {log}_{2} {FC}_{Live \_ 6 mo} AND {log}_{2} {FC}_{Frozen \_ 15 mo} \geq {log}_{2} {FC}_{Frozen \_ 6 mo}$$

Condition_{Frozen_snRNA}: Progressive increases from 6-month to 15-month timepoints in frozen-tissue snRNA-seq and non-decreases in live-tissue scRNA-seq

$${log}_{2} {FC}_{Frozen \_ 15 mo} > {log}_{2} {FC}_{Frozen \_ 6 mo} AND {log}_{2} {FC}_{Live \_ 15 mo} \geq {log}_{2} {FC}_{Live \_ 6 mo}$$

Condition_{Govaere}: Increased expression with disease stage

$${β}_{Govaere \_ limmatrend} > 0$$

Longitudinal Decrease program:
   Condition_{Live_scRNA}: Progressive decreases from 6-month to 15-month timepoints in live-tissue scRNA-seq and non-increases in frozen-tissue snRNA-seq

$${log}_{2} {FC}_{Live \_ 15 mo} < {log}_{2} {FC}_{Live \_ 6 mo} AND {log}_{2} {FC}_{Frozen \_ 15 mo} \leq {log}_{2} {FC}_{Frozen \_ 6 mo}$$

Condition_{Frozen_snRNA}: Progressive decreases from 6-month to 15-month timepoints in frozen-tissue snRNA-seq and non-increases in live-tissue scRNA-seq

$${log}_{2} {FC}_{Frozen \_ 15 mo} < {log}_{2} {FC}_{Frozen \_ 6 mo} AND {log}_{2} {FC}_{Live \_ 15 mo} \leq {log}_{2} {FC}_{Live \_ 6 mo}$$

Condition_{Govaere}: Decreased expression with disease stage

$${β}_{Govaere \_ limmatrend} < 0$$

Only the mouse live-tissue scRNA-seq dataset, mouse frozen-tissue snRNA-seq dataset, and Govaere et al. bulk RNA-seq dataset were used for stress adaptation gene program derivation. Importantly, none of the other datasets presented elsewhere herein (e.g., FIGS. 2P-2Q, 3D-3M, 6B-6H, 7G-7J, 10, 13, 17A, 14B-14Q, or 19-21) were used as part of gene program derivation, ensuring that these figures' analyses were conducted on “test sets” of entirely external, independent datasets. Module scores for each program and dataset were calculated using AddModuleScore in Seurat, and TCGA patient stratification and visualization were conducted with the survival and survminer packages in R.

### Comparison and Identification of Cancer Signature Induction During Stress Adaptation

To evaluate whether and which cancer and developmental phenotypes are mirrored in the spontaneous HCC tumors in the chronic metabolic stress mouse model, researchers began by conducting differential gene expression testing between tumor cells and hepatocytes in matched adjacent normal tissue. As a broad view of potential cancer phenotypes, researchers conducted gene set enrichment analysis using the fgsea R package, comparing the model's tumor differential expression against gene sets from: 1) MSigDB's “Chemical and Genetic Perturbations” (3,405 genesets spanning diverse prior studies' mutational and signaling perturbations), 2) mutation-specific mouse liver tumor models, and 3) liver development and regenerative states. Thus, all gene sets considered during gene set enrichment analysis were defined externally to this study, providing a complementary framework (to the previously-described adaptation program derivation) for uncovering phenotypes accentuated with tumorigenesis in this model.

Researchers then sought to understand which tumorigenesis-linked gene sets were also differentially regulated during hepatocytes' progressive stress adaptations (i.e., before tumorigenesis). Seurat's AddModuleScore was used to score each dataset for leading-edge genes from statistically-significantly enriched cancer and developmental gene sets. Thus, statistically-significant module score differences in mouse tumor-vs-adjacent normal comparisons (FIGS. 4A, 4F) reflect the statistically-significant gene set enrichment results on which the gene program is based; all significant differences in other datasets (FIGS. 4B-4E, 4G-4J, 14D-14E) represent “test set” results in entirely external datasets and independent extensions and connections to hepatocytes' progressive adaptations to chronic stress across species and cohorts.

### Intercellular Signaling Analyses

Potential intercellular signaling proteins that may drive hepatocytes' stress adaptation gene programs were inferred by applying NicheNet to live-tissue scRNA-seq data (which enables elevated proportions of immune cells as compared to frozen-tissue nuclei isolation-based datasets). Ligands were considered if detected in at least 10% of cells across any cell type. For each gene program, potential regulatory ligands were prioritized using NicheNet's predict_ligand_activities. To control for ligands that broadly regulate hepatocyte functions or non-specific computational inference, 100 random gene sets were derived with matched average expression levels to the input gene program of interest; ligands' regulatory potential scores were z-scored against a null background of their regulatory potentials for these random gene sets. Ligands were ranked by these regulatory potential z-scores, and only considered if their z-score was positive; a maximum of 8 ligands were retained for each gene program.

### Computational Methods for Longitudinal Epigenetic Alterations and Priming

Differential chromatin accessibility was calculated by pseudobulking snATAC-seq hepatocytes from each mouse, then calculating differential expression within each timepoint between high fat vs. control diet pseudobulked hepatocyte profiles using edgeR. chromVAR scores were calculated using the JASPAR2020 core motif collection and the Signac wrapper function RunChromVAR. Chromatin peak co-accessibility links were calculated using the Signac wrapper function run_cicero.

For higher-resolution inference of hepatocytes' epigenetic adaptation trajectories, pseudotime analyses were conducted. To avoid confounding effects of diet and time, separate pseudotime trajectories were derived for hepatocytes from high fat and control diet hepatocytes, following concepts in previous analyses on allergic inflammation and stem cell. Pseudotime trajectories were calculated for each diet condition using chromatin peaks that were differentially accessible with age within each diet condition. To control for varying cell counts across samples, each timepoint was downsampled to equal cell counts (matching the timepoint with the fewest cells). Latent semantic indexing and UMAP visualization (on LSI components 2 to 30) were implemented for each diet condition, and Slingshot was used to find pseudotime trajectories.

For epigenetic priming analyses, distal peaks were first linked to genes based on co-accessibility with peaks in a gene's promoter or gene body (only considering peaks within 100 kilobases of the gene and with Cicero co-accessibility score greater than 0.1). To identify genes whose putative distal chromatin regulators collectively exhibit differential accessibility, researchers then compared: 1) the observed distribution of differential accessibility fold-changes of actual co-accessible peaks, against, 2) 50 random background peaksets with matched average accessibility and GC bias. Finally, towards genes that may exhibit evidence of epigenetic priming in hepatocytes, genes were identified where:
1. Early chromatin accessibility changes exhibited the same directionality as transcriptional changes across longitudinal stress adaptation and tumorigenesis. Epigenetic comparison: 6-month high fat vs. control diet snATAC-seq.
   Transcriptional comparison: [15-month tumor vs. adjacent normal snRNA-seq]-[6-month high fat vs. control diet snRNA-seq].
2. Late chromatin accessibility changes (15-month high fat vs. control diet snATAC-seq) exhibited the same directionality as tumorigenesis transcriptional changes (15-month tumor vs. adjacent normal snRNA-seq).
   Epigenetic comparison: 15-month high fat vs. control diet snATAC-seq.
   Transcriptional comparison: 15-month tumor vs. adjacent normal snRNA-seq.
3. Tumorigenesis incurred a large shift in gene expression (15-month tumor vs. adjacent normal snRNA-seq).
   Transcriptional comparison: 15-month tumor vs. adjacent normal snRNA-seq
4. Early stress drove large changes in chromatin accessibility (6-month high fat vs. control diet snATAC-seq).
   Epigenetic comparison: 6-month high fat vs. control diet snATAC-seq differential accessibility fold-changes at gene-linked co-accessible peaks, relative to matched random background control.

### MATCHA: Multiomic Ascertainment of Transcriptional Causality Via Hierarchical Association

MATCHA seeks to prioritize transcription factors regulating arbitrary, user-specified gene programs, while leveraging multi-omic information on context-specific regulatory relationships (e.g., cell type- or tissue-specific gene-enhancer regulation).

As inputs, MATCHA accepts: 1) one or more arbitrary gene programs, 2) a TF motif database (e.g., JASPAR 2020), 3) multi-omic sc/snRNA-seq data, and optionally, 4) external datasets with relevance to the user's context (e.g., prior bulk or sc/snRNA-seq atlases).

As outputs, MATCHA provides: 1) prioritization scores of the predicted strength and directionality of a TF's regulatory effect on each arbitrary gene program (along with contributions of each input dataset to the overall prioritization score), 2) a bipartite network of which TFs regulate which gene programs (and in what direction, along with TF and gene program network centrality metrics), and 3) rankings of which TFs may co-regulate multiple gene programs simultaneously (if multiple gene programs were provided).

Towards inference of gene program-co-accessible enhancer-causal TF triads, MATCHA is based on the principle that robust, strong regulatory relationships should be reflected across multiple-omic layers and across datasets. Therefore, MATCHA follows the following steps:
1. If a particular TF regulates the user-specified gene program(s), then it is plausible that the regulatory relationship should be reflected in accessibility changes at program-associated chromatin regions containing the TF's motif. Therefore, MATCHA begins by identifying chromatin regions that are co-accessible with a gene's promoter or gene body (based on Cicero), then filters peaks based on genomic distance and co-accessibility strength towards plausible regulatory relationships. For each TF motif, MATCHA creates a program-specific motif score by evaluating accessibility at program-coaccessible peaks containing each TF motif (based on chromVAR). Finally, MATCHA calculates the correlation between the program-specific motif score and transcriptional expression of the gene program itself. In this way, MATCHA connects epigenetic alterations at program-linked, TF motif-containing peaks to transcriptional levels of the gene program.
2. If a particular TF regulates the user-specified gene program(s), then it is plausible that the regulatory relationship should be reflected in concordant changes between TF abundance and gene program transcriptions. Therefore, MATCHA calculates the correlation between expression level of each TF in the user-input TF motif database and transcriptional expression of the gene program, thereby connecting transcriptional TF abundance to transcriptional levels of the gene program.
3. If a particular TF regulates the user-specified gene program(s) robustly, then it is plausible that the regulatory relationship should be reflected not only in a single dataset, but across studies. Therefore, if the user provided multiple input datasets (whether bulk or single-cell), MATCHA calculates similar correlations as described above between gene program transcriptional level and either TF motif accessibility at program-linked peaks or TF transcriptional abundance. In this way, MATCHA connects each TF (motif) to transcriptional levels of the gene program across wide-ranging contexts (e.g., spanning species, disease severities, experimental designs, measurement technologies, etc.).
4. To create a single prioritization score for each TF that incorporates information across
   omic measurements and studies, MATCHA aggregates each correlation (as calculated in preceding steps). To account for different correlation distributions across
   omic measurement modalities and studies, correlations calculated in each previous step are scaled from −1 (most negative association between TF and gene program, indicative of repression) to 1 (most positive association between TF and gene program, indicative of activation). Scaled correlations are averaged, and TFs are ranked by the average of scaled correlations.
5. In addition to identifying which TFs regulate a particular gene program, it is important to understand whether TFs may co-regulate multiple gene programs, towards essential couplings of cellular phenotypes encapsulated by different programs or potential opportunities to disentangle otherwise-associated phenotypes. Therefore, if the user provided multiple input gene programs, MATCHA will create rankings of each TF's cross-dataset association with each gene program (as described in preceding steps), and calculate network centrality metrics for TFs and gene programs (e.g., out-degree for TFs to identify the number of gene programs that they strongly regulate, in-degree for gene programs to identify the number of TFs potentially regulating them). For concise summarization, MATCHA will filter to retain TFs that are ranked within the top TFs in at least a minimum number of gene programs (FIG. 5F generated with the top 10 TFs for each gene program, showing TFs linked to at least 2 gene programs). Optionally, users can further filter to retain only TFs whose regulatory relationships match observed transcriptional correlations between gene programs (e.g., if a given TF is linked to program₁ and program₂ which are in turn negatively correlated with each other at the transcriptional level, the TF must activate one of the programs but repress the other). In this way, MATCHA enables nomination and prioritization of TFs with strong regulatory effects on wide-ranging, but specific cellular phenotypes of particular interest to the user (e.g., this work's stress adaptation gene programs, with distinct temporal trends, functional enrichments, and prognostic stratification of human HCC survival).

### Other Embodiments

From the foregoing description, it will be apparent that variations and modifications may be made aspects and embodiments described herein to be adopted to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of selecting a subject for therapy, the method comprising detecting an increase in the expression or activity of a SOX4, RELB, and HMGCS2 polynucleotide or polypeptide; and administering to the subject an agent that inhibits SOX4, RelB, or HMGCS2 expression.

2. The method of claim 1, wherein the agent that inhibits Sox4 is an integrin avb6/8-blocking monoclonal antibody (mAb).

3. The method of claim 1, wherein the agent is an inhibitory polynucleotide that targets Sox4.

4. The method of claim 1, wherein the inhibitory polynucleotide is an siRNA, shRNA, or antisense polynucleotide.

5. The method of claim 1, wherein the agent that inhibits RelB is 1,25-Dihydroxyvitamin D3 or vinorelbine.

6. The method of claim 1, wherein the agent that inhibits RelB is an inhibitory polynucleotide.

7. The method of claim 1, wherein the agent that inhibits HMGCS2 is momilactone B or HMGCS2 inhibitor.

8. A method of treating a subject for hepatocellular carcinoma, the method comprising administering to the subject an agent that inhibits SOX4, RelB, or HMGCS2 expression.

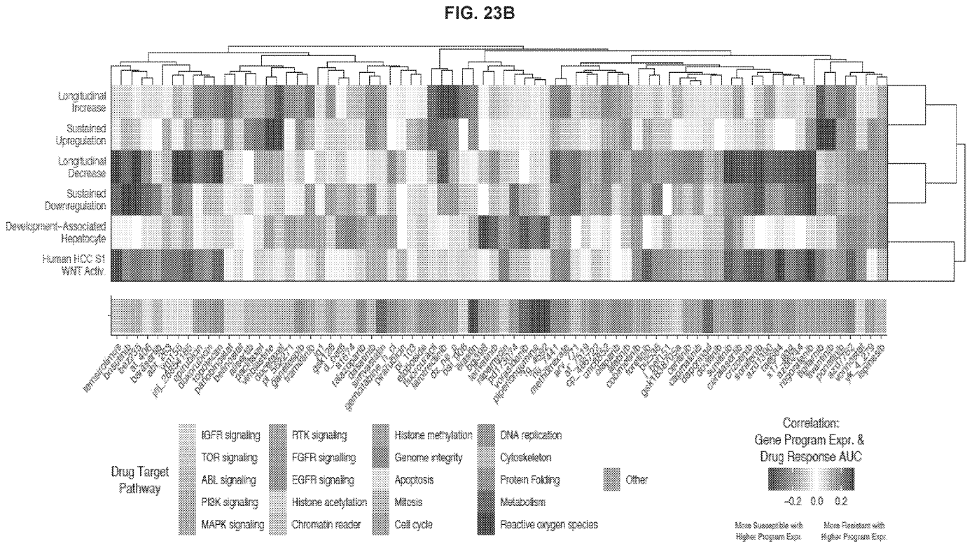

9. A method of treating a selected subject having or having a propensity to develop hepatocellular carcinoma, the method comprising administering to the subject an agent listed in FIG. 23A, wherein the subject is selected by detecting an increase in the expression of a marker or set of markers defined in Table 3 or Table 4 as having a longitudinal increase, sustained upregulation, longitudinal decrease, sustained downregulation, Development-associated hepatocyte, or Human HCC S1 Wnt Activator, and wherein the agent is selected as effective against a liver disease characterized by that marker or set of markers shown in FIG. 23A.

10. The method of claim 1, wherein the subject has metabolic dysfunction-associated steatotic liver disease (MASLD), progressive fibrosis, and/or liver failure.

11. The method of claim 1, wherein RELB and SOX4 drive metabolic adaptation and tumor priming phenotypes.

12. The method of claim 1, further comprising detecting increases in any one or more of the following markers or sets of markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, polynucleotides encoding those polypeptides;
viii. any marker listed as increased in Table 1 or any polypeptide listed as increased in Table 2A or 2B;
ix. a RELB, SOX4, JUND, ONECUT1, MAFF, RORC, DBP, or RXRA polypeptide or polynucleotide; and
x. a THRB, PPARA, NR1H4 polypeptide or polynucleotide.

13. The method of claim 1, further comprising detecting decreases in any one or more of the following sets of markers:
i. downregulation metabolic enzymes and secreted polypeptides Aspg, Hao1, Hamp, or polynucleotides encoding the polypeptides;
ii. suppressors of hepatocellular carcinoma and fetal hepatocyte-associated phenotypes iii. Gls2, Zbtb20, Esr1 polypeptides or polynucleotides encoding the polypeptides;
iii. decreases in hepatocyte secreted FGB and FABP1 polypeptides or polynucleotides encoding the polypeptides;
iv. polypeptides associated with hepatocyte cellular identity and function including NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides; and
v. any marker listed as decreased in Table 1 or any polypeptide listed as decreased in Table 2A or 2B.

14. A method of characterizing the prognosis of a subject having metabolic dysfunction-associated steatotic liver disease (MASLD), the method comprising detecting an increase in the expression or activity of a SOX4, RELB, and/or HMGCS2 polynucleotide or polypeptide.

15. The method of claim 14, wherein the increase in the expression or activity of a SOX4, RELB, and/or HMGCS2 polypeptide or polynucleotide is associated with the development of hepatocellular carcinoma.

16. The method of claim 14, further comprising detecting increases in any one or more of the following sets of markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, or polynucleotides encoding those polypeptides;
viii. WNT/β-catenin target Axin2;
ix. WNT-associated polypeptides Tbx3, Axin2, Lgr5, Notum or polynucleotides encoding those polypeptides;
x. HCC-linked polypeptides Spp1, Igf2r or polynucleotides encoding those polypeptides;
or polynucleotides encoding those polypeptides;
xi. a RELB, SOX4, JUND, ONECUT1, MAFF, RORC, DBP, or RXRA polypeptide or polynucleotide encoding those polypeptides; and
xii. a THRB, PPARA, NR1H4 polypeptide or polynucleotide encoding those polypeptides.

17. The method of claim 16, further comprising detecting decreases in any one or more of the following sets of markers:
i. downregulation metabolic enzymes and secreted polypeptides Aspg, Hao1, Hamp, or polynucleotides encoding the polypeptides;
ii. suppressors of hepatocellular carcinoma and fetal hepatocyte-associated phenotypes iii. Gls2, Zbtb20, Esr1 polypeptides or polynucleotides encoding the polypeptides;
iii. decreases in hepatocyte secreted FGB and FABP1 polypeptides or polynucleotides encoding the polypeptides;
iv. hepatocyte cellular identity polypeptides NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides; or
v. decreases in polypeptides associated with hepatocyte function Cps1, Pck1, Hamp, C4a, Pdia5 or polynucleotides encoding those polypeptides.

18. A panel of capture molecules comprising two or more molecules each of which specifically binds a SOX4, RELB, and/or HMGCS2 polypeptide or polynucleotide.

19. The panel of claim 18, wherein the panel further comprises one or more capture molecules each of which binds one of the following markers:
i. Krt8, Sox9, and Cd24a polypeptides or polynucleotides encoding those polypeptides;
ii. WNT-associated Tbx3, Axin2, Lgr5, Notum polypeptides or polynucleotides encoding those polypeptides;
iii. Hepatocellular Carcinoma linked polypeptides Spp1 and Igf2r or polynucleotides encoding those polypeptides;
iv. development-associated and regeneration-linked polypeptides CD24, CDKN1A, and LGR5 or polynucleotides encoding those polypeptides;
v. HCC-associated polypeptides p62/SQSTM1, CD151, LGALS1, CD74 or polynucleotides encoding those polypeptides;
vi. CD24, LGR5 and NKD1;
vii. genes with functional effects in HCC CD151, MDK, AIFM2, AKR1C2, ROBO1, or polynucleotides encoding those polypeptides;
viii. metabolic enzymes and secreted polypeptides Aspg, Hao1, Hamp, or polynucleotides encoding the polypeptides;
ix. suppressors of hepatocellular carcinoma and fetal hepatocyte-associated phenotypes Gls2, Zbtb20, Esr1 polypeptides or polynucleotides encoding the polypeptides;
x. hepatocyte secreted FGB and FABP1 polypeptides or polynucleotides encoding the polypeptides;
xi. polypeptides associated with hepatocyte cellular identity and function including NR1H4, PPARA, HNF4A, metabolic enzymes GPD1, AKR1D1, DPYD, EHHADH, and secreted protein products SERPINF2, PLG, FABP1, FGG, or polynucleotides encoding the polypeptides;
xii. decreases in polypeptides associated with hepatocyte function Cps1, Pck1, Hamp, C4a, Pdia5 or polynucleotides encoding those polypeptides;
xiii. a RELB, SOX4, JUND, ONECUT1, MAFF, RORC, DBP, or RXRA polypeptide or polynucleotide encoding those polypeptides; and
xiv. a THRB, PPARA, NR1H4 polypeptide or polynucleotide encoding those polypeptides.

20. A kit for characterizing metabolic dysfunction-associated steatotic liver disease (MASLD) or hepatocellular carcinoma in a subject, the kit comprising the capture molecule according to the method of claim 1.
